(19) United States
(12) Patent Application Publication
(10) Pub. No.: US 2024/0002480 A1
(43) Pub. Date: Jan. 4, 2024

(54) BROADLY NEUTRALIZING ANTIBODIES TO TICK-BORNE ENCEPHALITIS AND RELATED VIRUSES

(71) Applicant: The Rockefeller University, New York, NY (US)

(72) Inventors: Michel Nussenzweig, New York, NY (US); Davide F. Robbiani, New York, NY (US)

(73) Assignee: The Rockefeller University, New York, NY (US)

(21) Appl. No.: 18/253,981

(22) PCT Filed: Nov. 23, 2021
(86) PCT No.: PCT/US21/60595

§ 371 Date: May 23, 2023

Related U.S. Application Data
(60) Provisional application No. 63/118,461, filed on Nov. 25, 2020.

(51) Int. Cl. C07K 16/10; A61P 31/14; G01N 33/569

(52) U.S. Cl. CPC C07K 16/1081; A61P 31/14; G01N 33/56983; C07K 2317/34; C07K 2317/76; A61K 2039/505

(57) ABSTRACT

This disclosure provides novel broadly neutralizing anti-tick-borne encephalitis virus (TBEV) antibodies. The disclosed anti-TBEV antibodies represent a novel therapeutic strategy for preventing or treating diseases or infections caused by various tick-borne flaviviruses, including TBEV.

### CROSS-REFERENCE TO RELATED APPLICATIONS

The present application is a National Stage Application under 35 U.S.C. § 371 of International Application No. PCT/US2021/060595 filed Nov. 23, 2021, which claims benefit of U.S. Provisional Application No. 63/118,461 filed Nov. 25, 2020, the disclosures of all of which are hereby incorporated by reference in their entireties.

### STATEMENT REGARDING FEDERALLY FUNDED RESEARCH

This invention was made with government support under grant nos. P01-AI138398 and U19-AI111825 awarded by the National Institutes of Health. The government has certain rights in the invention.

### SEQUENCE LISTING

The instant application contains a Sequence Listing which has been submitted electronically in ASCII format and is hereby incorporated by reference in its entirety. Said ASCII copy, created on Jan. 17, 2022, is named 070413_20653_SL.txt and is 4,262,452 bytes in size.

### FIELD OF THE INVENTION

The present invention relates to antibodies directed to epitopes of tick-borne flaviviruses, including tick-borne encephalitis virus (TBEV).

### BACKGROUND

Tick-borne flaviviruses are responsible for a series of emerging infectious diseases, including fatal encephalitis. As with other flaviviruses, the TBEV envelope (E) is composed of three structural domains EDI-III. Tick-borne encephalitis virus (TBEV) is one of the seven flaviviruses transmitted by ticks causing human disease. Upwards of 10,000 cases per year are reported, with a trend for increased incidence in recent years and the emergence of the disease in new geographic regions.

The bite of an infected tick, or the consumption of unpasteurized milk from infected animals, causes a biphasic illness, which begins with a period of influenza-like symptoms followed by the development of neurological disease (tick-borne encephalitis or TBE). There is no specific therapy for TBE, and treatment is limited to supportive care. For those individuals that survive, long-term sequelae are common. Although TBEV vaccines are available, immunity requires regular boosting, and vaccination is less effective in the young and elderly. Vaccination requires administration of three separate doses spaced over up to two years, with booster doses recommended at intervals of 3-5 years. Moreover, breakthrough TBEV infection occurs despite vaccination.

Accordingly, there remains a strong need for anti-TBEV antibodies with improved neutralizing potency and breadth that are effective in prevention and treatment of diseases or infection caused by tick-borne flaviviruses, such as TBEV.

### SUMMARY

This disclosure addresses the need mentioned above in a number of aspects by providing broadly neutralizing anti-TBEV antibodies or antigen-binding fragments thereof.

In one aspect, this disclosure provides an isolated anti-TBEV antibody or antigen-binding fragment thereof that binds specifically to a TBEV antigen. In some embodiments, the TBEV antigen comprises a lateral ridge of domain III of the E protein (EDIII). In some embodiments, the antibody or antigen-binding fragment thereof is capable of neutralizing a plurality of TBEV strains.

In some embodiments, the antibody or antigen-binding fragment thereof comprises: (i) a heavy chain variable region having an amino acid sequence with at least 75% identity to one selected from those in Tables 2A-I, 3, and 4 or (ii) a light chain variable region having an amino acid sequence with at least 75% identity to one selected from those in Tables 2A-I, 3, and 4. In some examples, the antibody or antigen-binding fragment thereof comprises: (i) the three heavy chain CDRs (HCDRs 1-3) of one selected from those in Tables 2A-I, 3, and 4, and/or (ii) the three light chain CDRs (LCDRs 1-3) of one selected from those in Tables 2A-I, 3, and 4. In some embodiments, the antibody or antigen-binding fragment thereof comprises the six CDRs of one selected from those in Tables 2A-I, 3, and 4.

In some embodiments, the antibody or antigen-binding fragment thereof comprises: three heavy chain complementarity determining regions (HCDRs) (HCDR1, HCDR2, and HCDR3) of a heavy chain variable region having an amino acid sequence of SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, or 117; and three light chain CDRs (LCDR1, LCDR2, and LCDR3) of a light chain variable region having the amino acid sequence of SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, or 118.

In some embodiments, the antibody or antigen-binding fragment thereof comprises: a heavy chain variable region having an amino acid sequence with at least 75% identity to the amino acid sequence of SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, or 117; or having the amino acid sequence of SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, or 117; and a light chain variable region having an amino acid sequence with at least 75% identity to the amino acid sequence of SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, or 118; or having the amino acid sequence of SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, or 118.

In some embodiments, the antibody or antigen-binding fragment thereof comprises: a heavy chain variable region and a light chain variable region that comprise the respective amino acid sequences of SEQ ID NOs: 1-2, 3-4, 5-6, 7-8, 9-10, 11-12, 13-14, 15-16, 17-18, 19-20, 21-22, 23-24, 25-26, 27-28, 29-30, 31-32, 33-34, 35-36, 37-38, 39-40, 41-42, 43-44, 45-46, 47-48, 49-50, 51-52, 53-54, 55-56, 57-58, 59-60, 61-62, 63-64, 65-66, 67-68, 69-70, 71-72, 73-74, 75-76, 77-78, 79-80, 81-82, 83-84, 85-86, 87-88, 89-90, 91-92, 93-94, 95-96, 97-98, 99-100, 101-102, 103-104, 105-106, 107-108, 109-110, 111-112, 113-114, 115-116, or 117-118.

In some embodiments, the antibody is a multivalent antibody, e.g., a bivalent or bispecific antibody. In some embodiments, the antibody or the antigen-binding fragment thereof further comprises a variant Fc constant region. In some embodiments, the antibody is a monoclonal antibody. In some embodiments, the antibody is a chimeric antibody, a human antibody, a humanized antibody, or a humanized monoclonal antibody. In some embodiments, the antibody is a single-chain antibody, a Fab fragment or a Fab2 fragment.

In some embodiments, the antibody or antigen-binding fragment thereof is detectably labeled or conjugated to a toxin, a therapeutic agent, a polymer, a receptor, an enzyme, or a receptor ligand. In some embodiments, the polymer is polyethylene glycol (PEG).

In another aspect, this disclosure also provides a pharmaceutical composition comprising an antibody or antigen-binding fragment thereof described above and optionally a pharmaceutically acceptable carrier or excipient.

In some embodiments, the pharmaceutical composition comprises two or more of the antibodies or antigen-binding fragments thereof described above. In some example, each antibody or antigen-binding fragment thereof comprises (i) HCDRs1-3 and LCDRs1-3 of an antibody selected from those in Tables 2A-I, 3, and 4, or (ii) a heavy chain variable region and a light chain variable region that comprise the respective amino acid sequences of an antibody selected from those in Tables 2A-I, 3, and 4.

In some embodiments, the two or more of the antibody or antigen-binding fragment thereof comprise: (1) a first antibody set comprising: (i) a first antibody or antigen-binding fragment thereof comprising a heavy chain variable region and a light chain variable region comprising the respective amino acid sequences of a first antibody selected from those in Tables 2A-I, 3, and 4; and (ii) a second antibody or antigen-binding fragment thereof comprising a heavy chain variable region and a light chain variable region comprising the respective amino acid sequences of a second antibody selected from those in Tables 2A-I, 3, and 4; or (2) a second antibody set comprising: (a) a third antibody or antigen-binding fragment thereof comprising a heavy chain variable region and a light chain variable region comprising the respective amino acid sequences of antibody selected from those in Tables 2A-I, 3, and 4; and (b) a fourth antibody or antigen-binding fragment thereof comprising a heavy chain variable region and a light chain variable region comprising the respective amino acid sequences of an antibody selected from those in Tables 2A-I, 3, and 4, wherein the third antibody is different from the fourth antibody.

In some embodiments, the pharmaceutical composition further comprises a second therapeutic agent. In some embodiments, the second therapeutic agent comprises an anti-inflammatory agent or an antiviral agent. In some embodiments, the antiviral agent comprises: a nucleoside analog, a peptoid, an oligopeptide, a polypeptide, a protease inhibitor, a 3C-like protease inhibitor, a papain-like protease inhibitor, or an inhibitor of an RNA dependent RNA polymerase. In some embodiments, the antiviral agent may include: acyclovir, gancyclovir, vidarabine, foscarnet, cidofovir, amantadine, ribavirin, trifluorothymidine, zidovudine, didanosine, zalcitabine or an interferon. In some embodiments, the interferon is an interferon-α or an interferon-β.

Also within the scope of this disclosure is use of the described pharmaceutical composition in the preparation of a medicament for the diagnosis, prophylaxis, treatment, or combination thereof of a condition resulting from tick-borne flavivirus (e.g., TBEV) infection.

In another aspect, this disclosure also provides (i) a nucleic acid molecule encoding a polypeptide chain of the antibody or antigen-binding fragment thereof described above; (ii) a vector comprising the nucleic acid molecule as described; and (iii) a cultured host cell comprising the vector as described.

Also provided is a method for producing a polypeptide (e.g., an anti-TBEV antibody), comprising: (a) obtaining the cultured host cell as described; (b) culturing the cultured host cell in a medium under conditions permitting expression of a polypeptide encoded by the vector and assembling of an antibody or fragment thereof, and (c) purifying the antibody or fragment from the cultured cell or the medium of the cell.

In another aspect, this disclosure provides a kit comprising a pharmaceutically acceptable dose unit of the antibody or antigen-binding fragment thereof or a pharmaceutical composition as described above. Also within the scope of this disclosure is a kit for the diagnosis, prognosis or monitoring the treatment of tick-borne flavivirus (e.g., TBEV) in a subject, comprising: the antibody or antigen-binding fragment thereof as described; and a least one detection reagent that binds specifically to the antibody or antigen-binding fragment thereof.

In yet another aspect, this disclosure further provides a method of neutralizing a tick-borne encephalitis virus (e.g., TBEV) in a subject. The method comprises administering to a subject in need thereof a therapeutically effective amount of the antibody or antigen-binding fragment thereof or a therapeutically effective amount of the pharmaceutical composition, as described above.

In some embodiments, the method of neutralizing a tick-borne flavivirus (e.g., TBEV) in a subject comprises administering to a subject in need thereof a therapeutically effective amount of a first antibody or antigen-binding fragment thereof and a second antibody or antigen-binding fragment thereof of the antibody or antigen-binding fragment, as described above, wherein the first antibody or antigen-binding fragment thereof and the second antibody or antigen binding fragment thereof exhibit synergistic activity or a therapeutically effective amount of the pharmaceutical composition described above.

In yet another aspect, this disclosure additionally provides a method of preventing or treating tick-borne flavivirus (e.g., TBEV) infection. The method comprises administering to a subject in need thereof a therapeutically effective amount of the antibody or antigen-binding fragment thereof or a therapeutically effective amount of the pharmaceutical composition, as described above.

In some embodiments, the method comprises administering to a subject in need thereof a therapeutically effective amount of a first antibody or antigen-binding fragment thereof and a second antibody or antigen-binding fragment thereof of the antibody or antigen-binding fragment, as described above, wherein the first antibody or antigen-binding fragment thereof and the second antibody or antigen binding fragment thereof exhibit synergistic activity or a therapeutically effective amount of the pharmaceutical composition described above. In some embodiments, the first antibody or antigen-binding fragment thereof is administered before, after, or concurrently with the second antibody or antigen-binding fragment thereof.

In some embodiments, the first antibody or antigen-binding fragment thereof and the second antibody or antigen-binding fragment thereof can be any combinations of the antibody or antigen-binding fragment thereof comprising a heavy chain variable region and a light chain variable region that comprise the respective amino acid sequences of an antibody selected from those in Tables 2A-I, 3, and 4.

In some embodiments, the second therapeutic agent comprises an anti-inflammatory agent or an antiviral agent. In some embodiments, the antiviral agent comprises a nucleoside analog, a peptoid, an oligopeptide, a polypeptide, a protease inhibitor, a 3C-like protease inhibitor, a papain-like protease inhibitor, or an inhibitor of an RNA dependent RNA polymerase. In some embodiments, the antiviral agent may include: acyclovir, gancyclovir, vidarabine, foscarnet, cidofovir, amantadine, ribavirin, trifluorothymidine, zidovudine, didanosine, zalcitabine or an interferon. In some embodiments, the interferon is an interferon-α or an interferon-β.

In some embodiments, the antibody or antigen-binding fragment thereof is administered before, after, or concurrently with the second therapeutic agent or therapy. In some embodiments, the antibody or antigen-binding fragment thereof is administered to the subject intravenously, subcutaneously, or intraperitoneally. In some embodiments, the antibody or antigen-binding fragment thereof is administered prophylactically or therapeutically.

In another aspect, this disclosure further provides a method for detecting the presence of a tick-borne flavivirus (e.g., TBEV) in a sample comprising the steps of: (i) contacting a sample with the antibody or antigen-binding fragment thereof described above; and (ii) determining binding of the antibody or antigen-binding fragment to one or more tick-borne flavivirus (e.g., TBEV) antigens, wherein binding of the antibody to the one or more tick-borne flavivirus (e.g., TBEV) antigens is indicative of the presence of the tick-borne flavivirus (e.g., TBEV) in the sample. In some embodiments, the sample is a blood sample.

In some embodiments, the antibody or antigen-binding fragment thereof is conjugated to a label. In some embodiments, the step of detecting comprises contacting a secondary antibody with the antibody or antigen-binding fragment thereof and wherein the secondary antibody comprises a label. In some embodiments, the label includes a fluorescent label, a chemiluminescent label, a radiolabel, and an enzyme.

In some embodiments, the step of detecting comprises detecting fluorescence or chemiluminescence. In some embodiments, the step of detecting comprises a competitive binding assay or ELISA.

In some embodiments, the method further comprises binding the sample to a solid support. In some embodiments, the solid support includes microparticles, microbeads, magnetic beads, and an affinity purification column.

The foregoing summary is not intended to define every aspect of the disclosure, and additional aspects are described in other sections, such as the following detailed description. The entire document is intended to be related as a unified disclosure, and it should be understood that all combinations of features described herein are contemplated, even if the combination of features are not found together in the same sentence, or paragraph, or section of this document. Other features and advantages of the invention will become apparent from the following detailed description. It should be understood, however, that the detailed description and the specific examples, while indicating specific embodiments of the disclosure, are given by way of illustration only, because various changes and modifications within the spirit and scope of the disclosure will become apparent to those skilled in the art from this detailed description.

### BRIEF DESCRIPTION OF THE DRAWINGS

FIGS. 1A, 1B, 1C, 1D, and 1E are a set of diagrams showing the results of screening individuals for TBEV antibodies. FIG. 1A is a diagrammatic representation of the clinical course of tick-borne encephalitis. The approximate time of serum collection in yellow. FIG. 1B shows the results of TBEV EDIII IgG ELISA. The graph shows optical density measurement (Y axis) relative to a negative control serum for samples from 141 TBEV infected individuals, 10 TBEV vaccinees, and 168 random blood donors (1:500 dilution). The p values were calculated by one-way ANOVA followed by Tukey's test. Horizontal lines indicate the mean. FIG. 1C shows the results of TBEV RVP neutralization screening. Graph shows ranked serum neutralizing activity (1:600,000 dilution) against TBEV reporter virus particles (RVPs; average of duplicate wells) relative to no serum control. The orange box (bottom left) indicates the 28 best neutralizers of 141 TBEV infected individuals and 10 TBEV vaccinees tested. The p value is by two-tailed Mann-Whitney test. FIG. 1D shows TBEV RVP neutralization curves. The plot shows representative neutralization curves for each of the 28 most potent sera from FIG. 1C. Representative of 2 experiments, each performed in triplicate. Error bars indicate standard deviation. FIG. 1E shows ranked half-maximal serum neutralizing titers (NT₅₀) for the top 28 individuals. Average of two independent experiments. In FIGS. 1D and 1E, orange indicates the donors of peripheral blood mononuclear cells for antibody cloning.

FIGS. 2A, 2B, 2C, 2D, 2E, 2F, 2G, 2H, 2I, 2J, 2K, 2L, 2M, 2N, and 2O are a set of diagrams showing clinical correlations and serum neutralization in vaccinees. FIGS. 2A, 2B, 2C, 2D, 2E, and 2F show serum TBEV EDIII ELISA data (IgG) plotted against demographic and available clinical information. FIGS. 2G, 2H, 2I, and 2L show serum TBEV RVP neutralization data plotted against demographic and available clinical information. FIGS. 2C and 21 show severity of disease; FIGS. 2D and 2J show IgM titers (IP) measured at the time of hospitalization; FIGS. 2E and 2K show IgG titers (Vienna units/mL) measured at the time of hospitalization. Statistical significance was calculated for FIGS. 2A, 2B, 2D, 2E, 2G, 2H, 2J, and 2K using two-tailed p tests; for FIGS. 2L and 2F using Mann-Whitney tests; and for FIGS. 2C and 2I using one-way ANOVA with Tukey's test. FIG. 2M shows a correlation between serum TBEV EDIII ELISA (IgG) and RVP neutralization data. FIG. 2N shows TBEV RVP neutralization curves with sera from vaccinated PBMC donors. Representative of two experiments, in triplicates. Mean with standard deviation. FIG. 2O is a summary of serum NT₅₀s for all infected and vaccinated PBMC donors.

FIGS. 3A, 3B, 3C, and 3D are a set of diagrams showing anti-TBEV antibodies from infected and vaccinated individuals. FIG. 3A shows identification of TBEV-specific B cells from infected donors. Representative flow cytometry plots showing B cells binding to AF647- and PE-labeled TBEV EDIII in one control and six TBEV infected donors. Numbers indicate the percentage of double-positive B cells. The gating strategy is shown in FIG. 4A. FIG. 3B shows the clonal analysis of antibody sequences. Pie charts show the distribution of antibody sequences. The number in the center represents the total number of antibody sequences obtained. Colored or grey pie slices correspond to clonally related sequences, with the size of the slice proportional to the number of sequences. All blue slices are IGVH1-69; all red slices IGVH3-48/IGVK1-5. White slices correspond to antibody sequences that are not part of a clone (singlets). FIGS. 3C and 3D are the same as in FIGS. 3A and 3B but for one healthy control and three vaccinated donors. FIG. 3E shows antibody sequence relatedness. Circos plot shows sequences from all donors with color-coding as in FIGS. 3B and 3D. Connecting lines indicate antibodies that share IGH and IGL V and J genes. Purple, green, and grey lines connect related clones to each other, clones to singlets, and singlets to singlets, respectively.

FIGS. 4A, 4B, 4C, 4D, 4E, 4F, 4G, 4H, and 4I are a set of diagrams showing the sorting strategy and antibody sequence analysis. FIG. 4A shows the sorting strategy. Forward- and side-scatter were used to gate on single lymphocytes. Dump channel included CD3, CD8, CD14, CD16, and a viability dye. CD20⁺ B cells that failed to bind Ovalbumin (OVA⁻) but did bind to the TBEV EDIII bait coupled with both PE and AF647 fluorophores were purified. FIG. 4B shows the number of V gene somatic nucleotide mutations (left) and the amino acid length of the CDR3 (right) for each donor. FIG. 4C as in FIG. 4B, but for all donors combined. For FIGS. 4B and 4C, horizontal lines indicate the mean. FIG. 4 shows distribution of hydrophobicity GRAVY scores at the IGH CDR3 of antibodies from all donors combined and compared to human repertoire (Briney, B., et al., (2019) Nature 566, 393-397). FIG. 4E shows a bar graph depicting the frequency of V heavy chain gene usage in TBEV antibodies from infected donors compared to the human repertoire (Rubelt, F., et al., (2012) PLoS One 7, e49774). FIGS. 4F and 4G, as in FIG. 4E, but for V kappa and V lambda genes. In FIGS. 4E, 4F, and 4G, orange indicates anti-TBEV antibodies isolated in this study, while blue indicates control repertoire; p values calculated using two-tailed t-test with unequal variances. FIG. 4H shows sequence logos for antibody CDR3s from infected donors generated by WebLogo. The height of the stack indicates the sequence conservation at a given position, while the height of letters within the stack indicates the relative frequency of each amino acid at that position. FIG. 4I shows examples of highly similar antibody sequences found in across multiple donors. FIG. 4I discloses SEQ ID NOS 277, 4135, 2443, 1879, 1543, 6669, 643, 643, 643, 3787, 421, 2419, 1945, 355, 6670, 6670, 6670-6673, 1294, 1294, 3232, 3790, 424, 6674-6676, respectively, in order of column.

FIGS. 5A, 5B, 5C, 5D, 5E, 5F, and 5G are a set of diagrams showing identification of potent and broadly cross-reactive monoclonal antibodies. FIG. 5A shows TBEV^{WE} EDIII ELISA binding curves for 46 and 13 monoclonals from infected and vaccinated individuals, respectively. Data is representative of 2 experiments. The dotted line is 10-1074 isotype control. FIG. 5B shows a dot plot summarizing the EC₅₀ values for the antibodies in FIG. 5A to the three TBEV lineages EDIIIs: TBEV^{WE}, TBEV^{FE}, and TBEV^{SI}. Average of 2 experiments. The horizontal lines indicate the mean value. FIG. 5C shows RVP neutralization curves for the antibodies in FIG. 5A normalized to no antibody control. Data is representative of 2 experiments, each performed in triplicate. Error bars indicate standard deviation. FIG. 5D shows a dot plot summarizing the average half-maximal inhibitory concentration (IC₅₀) for TBEV^{WE} RVP neutralization by the antibodies in A. Average of two experiments. The horizontal line indicates the mean IC₅₀. No statistical difference was found by two-tailed Mann-Whitney test. FIGS. 5E and 5F show TBEV neutralization in vitro. In FIG. 5E, Curves represent virus neutralization by serially diluted antibodies. Representative of two independent experiments performed in octuplicates. In FIG. 5F, Representative immunofluorescence microscopy images of PS cells infected in the presence of the indicated antibodies. Green is viral antigen, and blue is cell nuclei. Scale bar indicates 200 μm. FIG. 5G shows cross-neutralization by anti-TBEV antibodies. The graph shows IC₅₀ for selected antibodies against RVPs corresponding to Powassan LB (POWV-LB), Powassan DTV (POWV-DTV), Kyasanur Forest Disease (KFDV), Langat (LGTV), louping Ill (LIV), and Omsk Hemorrhagic Fever viruses (OHFV). Average of two independent experiments. The horizontal line indicates the mean IC₅₀. In FIGS. 5A, 5B, 5C, 5D, and 5E, blue and red indicate infected donor-derived IGVH1-69/Kappa and IGVH3-48/IGVK1-5 antibodies; while purple indicates IGHV1-69/Kappa antibodies from vaccinated individuals. Antibodies T036 and T025 are shown in yellow and orange, respectively. In FIGS. 5B, 5D, and 5G closed circles and triangles correspond to antibodies derived from infected or vaccinated donors, respectively.

FIGS. 6A, 6B, 6C, 6D, 6E, 6F, 6G, 6H, and 6I are a set of diagrams showing antibody binding and neutralization. FIG. 6A shows ELISA binding curves to TBEV^{FE} and TBEV_{Si} EDIII for the 59 antibodies. Data are representative of two experiments. FIG. 6B shows screening for antibodies binding to a panel of tick-borne flavivirus EDIIIs, including Powassan LB (POWV-LB), Powassan Deer Tick (POWV-DTV), Kyasanur Forest Disease (KFDV), Langat (LGTV), louping Ill (LIV), and Omsk Hemorrhagic Fever viruses (OHFV). Antibodies were screened in duplicate at 1 μg/mL. Grey indicates binding over control. FIG. 6C shows screening for antibodies neutralization against RVPs corresponding to the same panel of tick-borne flaviviruses as in FIG. 6B. Antibodies were screened in triplicates at 1 g/mL. Grey indicates binding over control. FIGS. 6D, 6E, 6F, 6G, 6H, and 6I show neutralization curves of selected antibodies against tick-borne flavivirus RVPs other than TBEV. Representative of two experiments, in triplicates.

FIGS. 7A, 7B, 7C, 7D, and 7E are a set of diagrams showing that T036 enhances TBEV infection. FIG. 7A shows dose-dependent enhancement of TBEV RVP infection in the presence of T036 IgG, F(ab′)2, and F(ab). Representative of two experiments performed in triplicate. Error bars indicate standard deviation. FIG. 7B shows enhancement of virus titers. The plot shows Hypr-TBEV virus titers after incubation of PS cells for 24 or 48 hours in the presence of neutralizing antibody T038, enhancing antibody T036 or isotype control 10-1074. p values were calculated using one-way ANOVA and Tukey tests. The dashed line represents the limit of detection of the assay. FIG. 7C. Enhanced detection of viral antigen. Representative microscopy images of PS cells infected with Hypr-TBEV in the presence of the indicated amounts of T036, T038 or 10-1074 control. Scale bar indicates 200 m. FIGS. 7D and 7E. The fusion loop binding antibody 4G2 blocks the enhancement effect by T036. In FIG. 7D, TBEV RVP infection relative to no antibody control in the presence of antibody 4G2, T036 or 4G2 in combination with T036. Representative of two experiments. In FIG. 7E, cell plaques counts after infection of PS cells with Hypr-TBEV in the presence of 4G2, T036, or 4G2 and T036 in combination. For FIGS. 7D and 7E, the p values were calculated using one-way ANOVA and Tukey tests; error bars in D indicate the standard deviation of triplicates.

FIGS. 8A and 8B are a set of diagrams showing that T036 enhances TBEV infection. FIG. 8A is a plot showing TBEV Neudoerfl titers after infection of PS cells and incubation for 24 or 48 hours in the presence of T036, neutralizing antibody T038 or isotype control 10-1074. p values were calculated with one-way ANOVA and Tukey's test. FIG. 8B shows representative immunofluorescence microscopy images upon of PS cells with TBEV Neudoerfl in the presence of the indicated antibodies. Green is viral antigen, and blue is cell nuclei. Scale bar indicates 200 m.

FIGS. 9A, 9B, 9C, and 9D are a set of diagrams showing that the T025 antibody recognizes a lateral ridge epitope on TBEV EDIII that is exposed on the mature virus structure. FIG. 9A shows T025 recognition of the TBEV^{WE} EDIII. T025 interacts with the N-terminal region (EDI-EDIII hinge, the BC loop, and the DE loop) on TBEV^{WE} EDIII. FIG. 9B shows a T025 epitope. TBEV^{WE} EDIII residues with an atom within 4 Å of a residue in the T025 Fab are highlighted on a surface representation of the EDIII antigen. CDRH3 and CDRL3 are shown as ribbon backbone with stick side chains. FIG. 9C shows that T025 recognizes a similar epitope as the anti-TBEV mouse antibody 19/1786. The T025 epitope is shown in shades of orange; the 19/1786 epitope is outlined in a blue dashed line. Residues within the 19/1786 epitope, but not in the T025 epitope, are labeled. Epitopes are defined as residues that contain an atom within 4 Å of an atom in a residue on the antibody. FIG. 9D shows a surface representation of the cryo-EM structure of T025 (PDB 5O6A) shown with 5-fold, 3-fold, and 2-fold icosahedral symmetry operators at select vertices (left) with inset comparing binding poses of T025 and 19/1786 antibodies (right). Inset: close-up of the indicated portion (dotted box) of the cryo-EM structure of the viral surface interacting with the 19/1786 V_{H}V_{L} domains (PDB 506V) with the E protein domains labeled in red, yellow, and blue and the V_{H}V_{L} domains in teal and cyan. The T025-TBEV^{WE} EDIII crystal structure was docked onto a virion EDIII adjacent to an icosahedral 2-fold symmetry axis after alignment of the EDIII domains (RMSD=0.97 Å, 82 Cα atoms). The T025 V_{H}V_{L} binds EDIII with a similar pose as the 19/1786 V_{H}V_{L}.

FIGS. 10A and 10B are a set of diagrams showing prevention and therapy with T025. FIG. 10A shows that T025 is efficacious in pre-exposure prophylaxis. Mice were treated with T025 or 10-1074 (isotype control) 24 hours before infection with a lethal dose of TBEV-Hypr. Top, the histogram shows disease score overtime. Antibody dose is indicated on the right. Two independent experiments combined. Bottom, Kaplan Meyer survival curve. The p value was calculated with the Mantel-Cox test (p<0.0001). FIG. 10B shows that T025 protects mice when administered after infection. Mice were treated with 30 μg of T025 or control 10-1074 at 1, 3, or 5 days post infection (DPI). Three experiments combined and p<0.0001 for both +1 DPI and +3 DPI by Mantel-Cox test.

### DETAILED DESCRIPTION OF THE INVENTION

This disclosure describes anti-TBEV antibodies with unexpected broadly neutralizing activities. In addition to TBEV, these antibodies also neutralize other emerging tick-borne flaviviruses, including Langat, louping ill, Omsk hemorrhagic fever, Kyasanur forest disease, and Powassan viruses. Thus, the disclosed antibodies and antigen-binding fragments represent a novel therapeutic strategy for preventing or treating diseases or infections caused by various tick-borne flaviviruses, including TBEV.

### A. Broadly Neutralizing Anti-TBEV Antibodies

Antibodies

The invention disclosed herein involves broadly neutralizing anti-TBEV antibodies or antigen-binding fragments thereof. These antibodies refer to a class of neutralizing antibodies that neutralize multiple tick-borne flaviviruses and strains thereof. The antibodies are able to protect a subject prophylactically and therapeutically against a lethal challenge with a tick-borne flavivirus (e.g., TBEV).

In one aspect, this disclosure provides an isolated anti-TBEV antibody or antigen-binding fragment thereof that binds specifically to a tick-borne flavivirus (e.g., TBEV) antigen. In some embodiments, the antigen comprises a lateral ridge of EDIII.

Listed below in Tables 2A-I, 3, and 4 are representative amino acid and/or nucleic acid sequences of the heavy chain (HC) variable regions and light chain (LC) variable regions of exemplary anti-TBEV antibodies.

In some embodiments, the antibody or antigen-binding fragment thereof comprises: (i) a heavy chain variable region having an amino acid sequence with at least 75% (e.g., 75%, 50%, 85%, 90%, 92%, 94%, 95%, 96%, 97%, 98%, 99%) identity to one selected from the Tables 2A-I, 3, and 4 and (ii) a light chain variable region having an amino acid sequence with at least 75% (e.g., 75%, 50%, 85%, 90%, 92%, 94%, 95%, 96%, 97%, 98%, 99%) identity to one selected from Tables 2A-I, 3, and 4. In some embodiments, the antibody or antigen-binding fragment thereof comprises: (i) the three heavy chain CDRs (HCDRs 1-3) of one selected from those in Tables 2A-I, 3, and 4, and/or (ii) the three light chain CDRs (LCDRs 1-3) of one selected from those in Tables 2A-I, 3, and 4. In some embodiments, the antibody or antigen-binding fragment thereof comprises the six CDRs of one selected from those in Tables 2A-I, 3, and 4.

In some embodiments, the antibody or antigen-binding fragment thereof comprises: three heavy chain complementarity determining regions (HCDRs) (HCDR1, HCDR2, and HCDR3) of a heavy chain variable region having an amino acid sequence of SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, or 117; and three light chain CDRs (LCDR1, LCDR2, and LCDR3) of a light chain variable region having the amino acid sequence of SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, or 118.

In some embodiments, the antibody or antigen-binding fragment thereof comprises: a heavy chain variable region having an amino acid sequence with at least 75% identity to the amino acid sequence of SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, or 117; or having the amino acid sequence of SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, or 117; and a light chain variable region having an amino acid sequence with at least 75% identity to the amino acid sequence of SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, or 118; or having the amino acid sequence of SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, or 118.

In some embodiments, the antibody or antigen-binding fragment thereof comprises: a heavy chain variable region and a light chain variable region that comprise the respective amino acid sequences of SEQ ID NOs: 1-2, 3-4, 5-6, 7-8, 9-10, 11-12, 13-14, 15-16, 17-18, 19-20, 21-22, 23-24, 25-26, 27-28, 29-30, 31-32, 33-34, 35-36, 37-38, 39-40, 41-42, 43-44, 45-46, 47-48, 49-50, 51-52, 53-54, 55-56, 57-58, 59-60, 61-62, 63-64, 65-66, 67-68, 69-70, 71-72, 73-74, 75-76, 77-78, 79-80, 81-82, 83-84, 85-86, 87-88, 89-90, 91-92, 93-94, 95-96, 97-98, 99-100, 101-102, 103-104, 105-106, 107-108, 109-110, 111-112, 113-114, 115-116, or 117-118.

In some embodiments, the antibody or the antigen-binding fragment thereof further comprises a variant Fc constant region. In some embodiments, the antibody is a monoclonal antibody. In some embodiments, the antibody is a chimeric antibody, a humanized antibody, or a humanized monoclonal antibody. In some embodiments, the antibody is a single-chain antibody, a Fab fragment or a Fab2 fragment.

In some embodiments, the antibody or the antigen-binding fragment thereof further comprises a variant Fc constant region. The antibody can be a monoclonal antibody. In some embodiments, the antibody can be a chimeric antibody, a humanized antibody, or a humanized monoclonal antibody. In some embodiments, the antibody can be a single-chain antibody, Fab or Fab2 fragment.

In some embodiments, the antibody or antigen-binding fragment thereof can be detectably labeled or conjugated to a toxin, a therapeutic agent, a polymer (e.g., polyethylene glycol (PEG)), a receptor, an enzyme or a receptor ligand. For example, an antibody of the present invention may be coupled to a toxin (e.g., a tetanus toxin). Such antibodies may be used to treat animals, including humans, that are infected with the virus that is etiologically linked to a tick-borne flavivirus (e.g., TBEV).

In another example, an antibody of the present invention may be coupled to a detectable tag. Such antibodies may be used within diagnostic assays to determine if an animal, such as a human, is infected with a tick-borne flavivirus (e.g., TBEV). Examples of detectable tags include: fluorescent proteins (i.e., green fluorescent protein, red fluorescent protein, yellow fluorescent protein), fluorescent markers (i.e., fluorescein isothiocyanate, rhodamine, texas red), radiolabels (i.e., 3H, 32P, 125I), enzymes (i.e., β-galactosidase, horseradish peroxidase, β-glucuronidase, alkaline phosphatase), or an affinity tag (i.e., avidin, biotin, streptavidin). Methods to couple antibodies to a detectable tag are known in the art. Harlow et al., Antibodies: A Laboratory Manual, page 319 (Cold Spring Harbor Pub. 1988).

Fragment

In some embodiments, an antibody provided herein is an antibody fragment. Antibody fragments include, but are not limited to, Fab, Fab′, Fab′-SH, F(ab′)2, Fv, and single-chain Fv (scFv) fragments, and other fragments described below, e.g., diabodies, triabodies tetrabodies, and single-domain antibodies. For a review of certain antibody fragments, see Hudson et al., Nat. Med. 9:129-134 (2003). For a review of scFv fragments, see, e.g., Pluckthun, in The Pharmacology of Monoclonal Antibodies, vol. 113, Rosenburg and Moore eds., (Springer-Verlag, New York), pp. 269-315 (1994); see also WO 93/16185; and U.S. Pat. Nos. 5,571,894 and 5,587,458. For discussion of Fab and F(ab′)2 fragments comprising salvage receptor binding epitope residues and having increased in vivo half-life, see U.S. Pat. No. 5,869,046.

Diabodies are antibody fragments with two antigen-binding sites that may be bivalent or bispecific. See, for example, EP 404,097; WO 1993/01161; Hudson et al., Nat. Med. 9:129-134 (2003); and Hollinger et al., Proc. Natl. Acad. Sci. USA 90: 6444-6448 (1993). Triabodies and tetrabodies are also described in Hudson et al., Nat. Med. 9:129-134 (2003).

Single-domain antibodies are antibody fragments comprising all or a portion of the heavy chain variable domain or all or a portion of the light chain variable domain of an antibody. In some embodiments, a single-domain antibody is a human single-domain antibody (DOMANTIS, Inc., Waltham, Mass.; see, e.g., U.S. Pat. No. 6,248,516).

Antibody fragments can be made by various techniques, including but not limited to proteolytic digestion of an intact antibody as well as production by recombinant host cells (e.g., E. coli or phage), as described herein.

Chimeric and Humanized Antibodies

In some embodiments, an antibody provided herein is a chimeric antibody. Certain chimeric antibodies are described, e.g., in U.S. Pat. No. 4,816,567; and Morrison et al., Proc. Natl. Acad. Sci. USA, 81:6851-6855 (1984)). In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof.

In some embodiments, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which HVRs, e.g., CDRs, (or portions thereof) are derived from a non-human antibody, and FRs (or portions thereof) are derived from human antibody sequences. A humanized antibody optionally will also comprise at least a portion of a human constant region. In some embodiments, some FR residues in a humanized antibody are substituted with corresponding residues from a non-human antibody (e.g., the antibody from which the HVR residues are derived), e.g., to restore or improve antibody specificity or affinity.

Humanized antibodies and methods of making them are reviewed, e.g., in Almagro and Fransson, Front. Biosci. 13:1619-1633 (2008), and are further described, e.g., in Riechmann et al., Nature 332:323-329 (1988); Queen et al., Proc. Nat'l Acad. Sci. USA 86:10029-10033 (1989); U.S. Pat. Nos. 5,821,337, 7,527,791, 6,982,321, and 7,087,409; Kashmiri et al., Methods 36:25-34 (2005) (describing specificity determining region (SDR) grafting); Padlan, Mol. Immunol. 28:489-498 (1991) (describing “resurfacing”); Dall'Acqua et al., Methods 36:43-60 (2005) (describing “FR shuffling”); and Osbourn et al., Methods 36:61-68 (2005) and Klimka et al., Br. J. Cancer, 83:252-260 (2000) (describing the “guided selection” approach to FR shuffling).

Human framework regions that may be used for humanization include but are not limited to: framework regions selected using the “best-fit” method (see, e.g., Sims et al. J. Immunol. 151:2296 (1993)); framework regions derived from the consensus sequence of human antibodies of a particular subgroup of light or heavy chain variable regions (see, e.g., Carter et al. Proc. Natl. Acad. Sci. USA, 89:4285 (1992); and Presta et al. J. Immunol., 151:2623 (1993)); human mature (somatically mutated) framework regions or human germline framework regions (see, e.g., Almagro and Fransson, Front. Biosci. 13:1619-1633 (2008)); and framework regions derived from screening FR libraries (see, e.g., Baca et al., J. Biol. Chem. 272:10678-10684 (1997) and Rosok et al., J. Biol. Chem. 271:22611-22618 (1996)).

Human Antibodies

In some embodiments, an antibody provided herein is a human antibody. Human antibodies can be produced using various techniques known in the art or using techniques described herein. Human antibodies are described generally in van Dijk and van de Winkel, Curr. Opin. Pharmacol. 5: 368-74 (2001) and Lonberg, Curr. Opin. Immunol. 20:450-459 (2008).

Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For review of methods for obtaining human antibodies from transgenic animals, see Lonberg, Nat. Biotech. 23:1117-1125 (2005). See also, e.g., U.S. Pat. Nos. 6,075,181 and 6,150,584 describing XENOMOUSE technology; U.S. Pat. No. 5,770,429 describing HUMAB technology; U.S. Pat. No. 7,041,870 describing K-M MOUSE technology, and U.S. Patent Application Publication No. US 2007/0061900, describing VELOCIMOUSE technology). Human variable regions from intact antibodies generated by such animals may be further modified, e.g., by combining with a different human constant region.

Human antibodies can also be made by hybridoma-based methods. Human myeloma and mouse-human heteromyeloma cell lines for the production of human monoclonal antibodies have been described. (See, e.g., Kozbor J. Immunol., 133: 3001 (1984); Brodeur et al., Monoclonal Antibody Production Techniques and Applications, pp. 51-63 (Marcel Dekker, Inc., New York, 1987); and Boerner et al., J. Immunol., 147: 86 (1991).) Human antibodies generated via human B-cell hybridoma technology are also described in Li et al., Proc. Natl. Acad. Sci. USA, 103:3557-3562 (2006). Additional methods include those described, for example, in U.S. Pat. No. 7,189,826 (describing production of monoclonal human IgM antibodies from hybridoma cell lines) and Ni, Xiandai Mianyixue, 26(4):265-268 (2006) (describing human-human hybridomas). Human hybridoma technology (Trioma technology) is also described in Vollmers and Brandlein, Histology and Histopathology, 20(3):927-937 (2005) and Vollmers and Brandlein, Methods and Findings in Experimental and Clinical Pharmacology, 27(3):185-91 (2005).

Human antibodies may also be generated by isolating Fv clone variable domain sequences selected from human-derived phage display libraries. Such variable domain sequences may then be combined with a desired human constant domain. Techniques for selecting human antibodies from antibody libraries are described below.

Antibodies of the invention may be isolated by screening combinatorial libraries for antibodies with the desired activity or activities. For example, a variety of methods are known in the art for generating phage display libraries and screening such libraries for antibodies possessing the desired binding characteristics. Such methods are reviewed, e.g., in Hoogenboom et al., in Methods in Molecular Biology 178:1-37 (O'Brien et al., ed., Human Press, Totowa, N.J., 2001) and further described, e.g., in the McCafferty et al., Nature 348:552-554; Clackson et al., Nature 352: 624-628 (1991); Marks et al., J. Mol. Biol. 222: 581-597 (1992); Marks and Bradbury, in Methods in Molecular Biology 248:161-175 (Lo, ed., Human Press, Totowa, N.J., 2003); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132 (2004).

In certain phage display methods, repertoires of VH and VL genes are separately cloned by polymerase chain reaction (PCR) and recombined randomly in phage libraries, which can then be screened for antigen-binding phage as described in Winter et al., Ann. Rev. Immunol., 12: 433-455 (1994). Phage typically displays antibody fragments, either as scFv fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self-antigens without any immunization as described by Griffiths et al., EMBO J, 12: 725-734 (1993). Finally, naive libraries can also be made synthetically by cloning unrearranged V-gene segments from stem cells and using PCR primers containing random sequences to encode the highly variable CDR3 regions and to accomplish rearrangement in vitro, as described by Hoogenboom and Winter, J. Mol. Biol., 227: 381-388 (1992). Patent publications describing human antibody phage libraries include, for example, U.S. Pat. No. 5,750,373, and US Patent Publication Nos. 2005/0079574, 2005/0119455, 2005/0266000, 2007/0117126, 2007/0160598, 2007/0237764, 2007/0292936, and 2009/0002360. Antibodies or antibody fragments isolated from human antibody libraries are considered human antibodies or human antibody fragments herein.

Variants

In some embodiments, amino acid sequence variants of the antibodies provided herein are contemplated. For example, it may be desirable to improve the binding affinity and/or other biological properties of the antibody. Amino acid sequence variants of an antibody may be prepared by introducing appropriate modifications into the nucleotide sequence encoding the antibody or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen binding.

Substitution, Insertion, and Deletion Variants

In some embodiments, antibody variants having one or more amino acid substitutions are provided. Sites of interest for substitutional mutagenesis include the HVRs and FRs. Conservative substitutions are defined herein. Amino acid substitutions may be introduced into an antibody of interest and the products screened for a desired activity, e.g., retained/improved antigen binding, decreased immunogenicity, or improved antibody-dependent cell-mediated cytotoxicity (ADCC) and complement-dependent cytotoxicity (CDC).

Accordingly, an antibody of the invention can comprise one or more conservative modifications of the CDRs, heavy chain variable region, or light variable regions described herein. A conservative modification or functional equivalent of a peptide, polypeptide, or protein disclosed in this invention refers to a polypeptide derivative of the peptide, polypeptide, or protein, e.g., a protein having one or more point mutations, insertions, deletions, truncations, a fusion protein, or a combination thereof. It substantially retains the activity of the parent peptide, polypeptide, or protein (such as those disclosed in this invention). In general, a conservative modification or functional equivalent is at least 60% (e.g., any number between 60% and 100%, inclusive, e.g., 60%, 70%, 75%, 80%, 85%, 90%, 95%, 96%, 97%, 98%, and 99%) identical to a parent. Accordingly, within the scope of this invention are heavy chain variable region or light variable regions having one or more point mutations, insertions, deletions, truncations, a fusion protein, or a combination thereof, as well as antibodies having the variant regions.

As used herein, the percent homology between two amino acid sequences is equivalent to the percent identity between the two sequences. The percent identity between the two sequences is a function of the number of identical positions shared by the sequences (i.e., % homology=# of identical positions/total # of positions×100), taking into account the number of gaps and the length of each gap, which need to be introduced for the optimal alignment of the two sequences. The comparison of sequences and determination of percent identity between two sequences can be accomplished using a mathematical algorithm, as described in the non-limiting examples below.

The percent identity between two amino acid sequences can be determined using the algorithm of E. Meyers and W. Miller (Comput. Appl. Biosci., 4:11-17 (1988)) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol. Biol. 48:444-453 (1970)) algorithm, which has been incorporated into the GAP program in the GCG software package (available at www.gcg.com), using either a Blossum 62 matrix or a PAM250 matrix, and a gap weight of 16, 14, 12, 10, 8, 6, or 4 and a length weight of 1, 2, 3, 4, 5, or 6.

Additionally or alternatively, the protein sequences of the present invention can further be used as a “query sequence” to perform a search against public databases to, for example, identify related sequences. Such searches can be performed using the XBLAST program (version 2.0) of Altschul, et al. (1990) J. Mol. Biol. 215:403-10. BLAST protein searches can be performed with the XBLAST program, score=50, wordlength=3 to obtain amino acid sequences homologous to the antibody molecules of the invention. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used. (See www.ncbi.nlm.nih.gov).

As used herein, the term “conservative modifications” refers to amino acid modifications that do not significantly affect or alter the binding characteristics of the antibody containing the amino acid sequence. Such conservative modifications include amino acid substitutions, additions, and deletions. Modifications can be introduced into an antibody of the invention by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include: (i) amino acids with basic side chains (e.g., lysine, arginine, histidine), (ii) acidic side chains (e.g., aspartic acid, glutamic acid), (iii) uncharged polar side chains (e.g., glycine, asparagine, glutamine, serine, threonine, tyrosine, cysteine, tryptophan), (iv) nonpolar side chains (e.g., alanine, valine, leucine, isoleucine, proline, phenylalanine, methionine), (v) beta-branched side chains (e.g., threonine, valine, isoleucine), and (vi) aromatic side chains (e.g., tyrosine, phenylalanine, tryptophan, histidine).

Non-conservative substitutions will entail exchanging a member of one of these classes for another class.

An exemplary substitutional variant is an affinity matured antibody, which may be conveniently generated, e.g., using phage display-based affinity maturation techniques such as those described in, e.g., Hoogenboom et al., in Methods in Molecular Biology 178:1-37 (O'Brien et al., ed., Human Press, Totowa, N.J., (2001). Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g., for ADEPT) or a polypeptide which increases the serum half-life of the antibody.

Glycosylation Variants

In some embodiments, an antibody provided herein is altered to increase or decrease the extent to which the antibody is glycosylated. Addition or deletion of glycosylation sites to an antibody may be conveniently accomplished by altering the amino acid sequence such that one or more glycosylation sites are created or removed.

For example, an aglycoslated antibody can be made (i.e., the antibody lacks glycosylation). Glycosylation can be altered to, for example, increase the affinity of the antibody for antigen. Such carbohydrate modifications can be accomplished by, for example, altering one or more sites of glycosylation within the antibody sequence. For example, one or more amino acid substitutions can be made that result in elimination of one or more variable region framework glycosylation sites to thereby eliminate glycosylation at that site. Such aglycosylation may increase the affinity of the antibody for antigen. Such an approach is described in further detail in U.S. Pat. Nos. 5,714,350 and 6,350,861 by Co et al.

Glycosylation of the constant region on N297 may be prevented by mutating the N297 residue to another residue, e.g., N297A, and/or by mutating an adjacent amino acid, e.g., 298 to thereby reduce glycosylation on N297.

Additionally or alternatively, an antibody can be made that has an altered type of glycosylation, such as a hypofucosylated antibody having reduced amounts of fucosyl residues or an antibody having increased bisecting GlcNac structures. Such altered glycosylation patterns have been demonstrated to increase the ADCC ability of antibodies. Such carbohydrate modifications can be accomplished by, for example, expressing the antibody in a host cell with altered glycosylation machinery. Cells with altered glycosylation machinery have been described in the art and can be used as host cells in which to express recombinant antibodies described herein to thereby produce an antibody with altered glycosylation. For example, EP 1,176,195 by Hanai et al. describes a cell line with a functionally disrupted FUT8 gene, which encodes a fucosyltransferase, such that antibodies expressed in such a cell line exhibit hypofucosylation. PCT Publication WO 03/035835 by Presta describes a variant Chinese Hamster Ovary cell line, Led 3 cells, with reduced ability to attach fucose to Asn(297)-linked carbohydrates, also resulting in hypofucosylation of antibodies expressed in that host cell (see also Shields, R. L. et al. (2002) J. Biol. Chem. 277:26733-26740). PCT Publication WO 99/54342 by Umana et al. describes cell lines engineered to express glycoprotein-modifying glycosyltransferases (e.g., beta(1,4)-N-acetylglucosaminyltransferase III (GnTIII)) such that antibodies expressed in the engineered cell lines exhibit increased bisecting GlcNac structures, which result in increased ADCC activity of the antibodies (see also Umana et al. (1999) Nat. Biotech. 17: 176-180).

Fc Region Variants

The variable regions of the antibody described herein can be linked (e.g., covalently linked or fused) to an Fc, e.g., an IgG1, IgG2, IgG3 or IgG4 Fc, which may be of any allotype or isoallotype, e.g., for IgG1: G1m, G1m1(a), G1m2(x), G1m3(f), G1m17(z); for IgG2: G2m, G2m23(n); for IgG3: G3m, G3m21(g1), G3m28(g5), G3 m11(b0), G3m5(b1), G3m13(b3), G3m14(b4), G3m10(b5), G3m15(s), G3m16(t), G3m6(c3), G3m24(c5), G3m26(u), G3m27(v); and for K: Km, Km1, Km2, Km3 (see, e.g., Jefferies et al. (2009) mAbs 1: 1). In some embodiments, the antibody variable regions described herein are linked to an Fe that binds to one or more activating Fc receptors (FcγI, Fcγlla or FcγIIIa), and thereby stimulate ADCC and may cause T cell depletion. In some embodiments, the antibody variable regions described herein are linked to an Fc that causes depletion.

In some embodiments, the antibody variable regions described herein may be linked to an Fc comprising one or more modifications, typically to alter one or more functional properties of the antibody, such as serum half-life, complement fixation, Fc receptor binding, and/or antigen-dependent cellular cytotoxicity. Furthermore, an antibody described herein may be chemically modified (e.g., one or more chemical moieties can be attached to the antibody) or be modified to alter its glycosylation, to alter one or more functional properties of the antibody. The numbering of residues in the Fc region is that of the EU index of Kabat.

The Fc region encompasses domains derived from the constant region of an immunoglobulin, preferably a human immunoglobulin, including a fragment, analog, variant, mutant or derivative of the constant region. Suitable immunoglobulins include IgG1, IgG2, IgG3, IgG4, and other classes such as IgA, IgD, IgE, and IgM. The constant region of an immunoglobulin is defined as a naturally-occurring or synthetically-produced polypeptide homologous to the immunoglobulin C-terminal region and can include a CH1 domain, a hinge, a CH2 domain, a CH3 domain, or a CH4 domain, separately or in combination. In some embodiments, an antibody of this invention has an Fc region other than that of a wild type IgA1. The antibody can have an Fc region from that of IgG (e.g., IgG1, IgG2, IgG3, and IgG4) or other classes such as IgA2, IgD, IgE, and IgM. The Fc can be a mutant form of IgA1.

The constant region of an immunoglobulin is responsible for many important antibody functions, including Fc receptor (FcR) binding and complement fixation. There are five major classes of heavy chain constant region, classified as IgA, IgG, IgD, IgE, IgM, each with characteristic effector functions designated by isotype. For example, IgG is separated into four subclasses known as IgG1, IgG2, IgG3, and IgG4.

Ig molecules interact with multiple classes of cellular receptors. For example, IgG molecules interact with three classes of Fcγ receptors (FcγR) specific for the IgG class of antibody, namely FcγRI, FcγRII, and FcγRIIL. The important sequences for the binding of IgG to the FcγR receptors have been reported to be located in the CH2 and CH3 domains. The serum half-life of an antibody is influenced by the ability of that antibody to bind to an FcR.

In some embodiments, the Fc region is a variant Fc region, e.g., an Fc sequence that has been modified (e.g., by amino acid substitution, deletion and/or insertion) relative to a parent Fc sequence (e.g., an unmodified Fc polypeptide that is subsequently modified to generate a variant), to provide desirable structural features and/or biological activity. For example, one may make modifications in the Fc region in order to generate an Fc variant that (a) has increased or decreased ADCC, (b) increased or decreased CDC, (c) has increased or decreased affinity for C1q and/or (d) has increased or decreased affinity for an Fc receptor relative to the parent Fc. Such Fc region variants will generally comprise at least one amino acid modification in the Fc region. Combining amino acid modifications is thought to be particularly desirable. For example, the variant Fc region may include two, three, four, five, or more substitutions therein, e.g., of the specific Fc region positions identified herein.

A variant Fc region may also comprise a sequence alteration wherein amino acids involved in disulfide bond formation are removed or replaced with other amino acids. Such removal may avoid reaction with other cysteine-containing proteins present in the host cell used to produce the antibodies described herein. Even when cysteine residues are removed, single chain Fc domains can still form a dimeric Fc domain that is held together non-covalently. In other embodiments, the Fc region may be modified to make it more compatible with a selected host cell. For example, one may remove the PA sequence near the N-terminus of a typical native Fc region, which may be recognized by a digestive enzyme in E. coli such as proline iminopeptidase. In other embodiments, one or more glycosylation sites within the Fc domain may be removed. Residues that are typically glycosylated (e.g., asparagine) may confer cytolytic response. Such residues may be deleted or substituted with unglycosylated residues (e.g., alanine). In other embodiments, sites involved in interaction with complement, such as the C1q binding site, may be removed from the Fc region. For example, one may delete or substitute the EKK sequence of human IgG1. In some embodiments, sites that affect binding to Fc receptors may be removed, preferably sites other than salvage receptor binding sites. In other embodiments, an Fc region may be modified to remove an ADCC site. ADCC sites are known in the art; see, for example, Molec. Immunol. 29 (5): 633-9 (1992) with regard to ADCC sites in IgG1. Specific examples of variant Fe domains are disclosed, for example, in WO 97/34631 and WO 96/32478.

In one embodiment, the hinge region of Fc is modified such that the number of cysteine residues in the hinge region is altered, e.g., increased or decreased. This approach is described further in U.S. Pat. No. 5,677,425 by Bodmer et al. The number of cysteine residues in the hinge region of Fc is altered to, for example, facilitate assembly of the light and heavy chains or to increase or decrease the stability of the antibody. In one embodiment, the Fc hinge region of an antibody is mutated to decrease the biological half-life of the antibody. More specifically, one or more amino acid mutations are introduced into the CH2-CH3 domain interface region of the Fc-hinge fragment such that the antibody has impaired Staphylococcal protein A (SpA) binding relative to native Fc-hinge domain SpA binding. This approach is described in further detail in U.S. Pat. No. 6,165,745 by Ward et al.

In yet other embodiments, the Fc region is altered by replacing at least one amino acid residue with a different amino acid residue to alter the effector function(s) of the antibody. For example, one or more amino acids selected from amino acid residues 234, 235, 236, 237, 297, 318, 320, and 322 can be replaced with a different amino acid residue such that the antibody has an altered affinity for an effector ligand but retains the antigen-binding ability of the parent antibody. The effector ligand to which affinity is altered can be, for example, an Fc receptor or the CI component of complement. This approach is described in further detail in U.S. Pat. Nos. 5,624,821 and 5,648,260, both by Winter et al.

In another example, one or more amino acids selected from amino acid residues 329, 331, and 322 can be replaced with a different amino acid residue such that the antibody has altered C1q binding and/or reduced or abolished CDC. This approach is described in further detail in U.S. Pat. No. 6,194,551 by Idusogie et al.

In another example, one or more amino acid residues within amino acid positions 231 and 239 are altered to thereby alter the ability of the antibody to fix complement. This approach is described further in PCT Publication WO 94/29351 by Bodmer et al.

In yet another example, the Fc region may be modified to increase ADCC and/or to increase the affinity for an Fcγ receptor by modifying one or more amino acids at the following positions: 234, 235, 236, 238, 239, 240, 241, 243, 244, 245, 247, 248, 249, 252, 254, 255, 256, 258, 262, 263, 264, 265, 267, 268, 269, 270, 272, 276, 278, 280, 283, 285, 286, 289, 290, 292, 293, 294, 295, 296, 298, 299, 301, 303, 305, 307, 309, 312, 313, 315, 320, 322, 324, 325, 326, 327, 329, 330, 331, 332, 333, 334, 335, 337, 338, 340, 360, 373, 376, 378, 382, 388, 389, 398, 414, 416, 419, 430, 433, 434, 435, 436, 437, 438 or 439. Exemplary substitutions include 236A, 239D, 239E, 268D, 267E, 268E, 268F, 324T, 332D, and 332E. Exemplary variants include 239D/332E, 236A/332E, 236A/239D/332E, 268F/324T, 267E/268F, 267E/324T, and 267E/268F7324T. Other modifications for enhancing FcγR and complement interactions include but are not limited to substitutions 298A, 333A, 334A, 326A, 2471, 339D, 339Q, 280H, 290S, 298D, 298V, 243L, 292P, 300L, 396L, 305I, and 396L. These and other modifications are reviewed in Strohl, 2009, Current Opinion in Biotechnology 20:685-691.

Fc modifications that increase binding to an Fcγ receptor include amino acid modifications at any one or more of amino acid positions 238, 239, 248, 249, 252, 254, 255, 256, 258, 265, 267, 268, 269, 270, 272, 279, 280, 283, 285, 298, 289, 290, 292, 293, 294, 295, 296, 298, 301, 303, 305, 307, 312, 315, 324, 327, 329, 330, 335, 337, 3338, 340, 360, 373, 376, 379, 382, 388, 389, 398, 414, 416, 419, 430, 434, 435, 437, 438 or 439 of the Fc region, wherein the numbering of the residues in the Fc region is that of the EU index as in abat (WO00/42072).

Other Fc modifications that can be made to Fcs are those for reducing or ablating binding to FcγR and/or complement proteins, thereby reducing or ablating Fc-mediated effector functions such as ADCC, antibody-dependent cellular phagocytosis (ADCP), and CDC. Exemplary modifications include but are not limited to substitutions, insertions, and deletions at positions 234, 235, 236, 237, 267, 269, 325, and 328, wherein numbering is according to the EU index. Exemplary substitutions include but are not limited to 234G, 235G, 236R, 237K, 267R, 269R, 325L, and 328R, wherein numbering is according to the EU index. An Fc variant may comprise 236R/328R. Other modifications for reducing FcγR and complement interactions include substitutions 297A, 234A, 235A, 237A, 318A, 228P, 236E, 268Q, 309L, 330S, 331S, 220S, 226S, 229S, 238S, 233P, and 234V, as well as removal of the glycosylation at position 297 by mutational or enzymatic means or by production in organisms such as bacteria that do not glycosylate proteins. These and other modifications are reviewed in Strohl, 2009, Current Opinion in Biotechnology 20:685-691.

Optionally, the Fc region may comprise a non-naturally occurring amino acid residue at additional and/or alternative positions known to one skilled in the art (see, e.g., U.S. Pat. Nos. 5,624,821; 6,277,375; 6,737,056; 6,194,551; 7,317,091; 8,101,720; WO00/42072; WO01/58957; WO02/06919; WO04/016750; WO04/029207; WO04/035752; WO04/074455; WO04/099249; WO04/063351; WO05/070963; WO05/040217, WO05/092925 and WO06/020114).

Fc variants that enhance affinity for an inhibitory receptor FcγRIIb may also be used. Such variants may provide an Fc fusion protein with immune-modulatory activities related to FcγRIIb cells, including, for example, B cells and monocytes. In one embodiment, the Fc variants provide selectively enhanced affinity to FcγRIIb relative to one or more activating receptors. Modifications for altering binding to FcγRIIb include one or more modifications at a position selected from the group consisting of 234, 235, 236, 237, 239, 266, 267, 268, 325, 326, 327, 328, and 332, according to the EU index. Exemplary substitutions for enhancing FcγRIIb affinity include but are not limited to 234D, 234E, 234F, 234W, 235D, 235F, 235R, 235Y, 236D, 236N, 237D, 237N, 239D, 239E, 266M, 267D, 267E, 268D, 268E, 327D, 327E, 328F, 328W, 328Y, and 332E. Exemplary substitutions include 235Y, 236D, 239D, 266M, 267E, 268D, 268E, 328F, 328W, and 328Y. Other Fc variants for enhancing binding to FcγRllb include 235Y/267E, 236D/267E, 239D/268D, 239D/267E, 267E/268D, 267E/268E, and 267E/328F.

The affinities and binding properties of an Fc region for its ligand may be determined by a variety of in vitro assay methods (biochemical or immunological based assays) known in the art, including, but not limited to, equilibrium methods (e.g., ELISA, or radioimmunoassay), or kinetics (e.g., BIACORE analysis), and other methods such as indirect binding assays, competitive inhibition assays, fluorescence resonance energy transfer (FRET), gel electrophoresis and chromatography (e.g., gel filtration). These and other methods may utilize a label on one or more of the components being examined and/or employ a variety of detection methods, including, but not limited to, chromogenic, fluorescent, luminescent, or isotopic labels. A detailed description of binding affinities and kinetics can be found in Paul, W. E., ed., Fundamental Immunology, 4th Ed., Lippincott-Raven, Philadelphia (1999), which focuses on antibody-immunogen interactions.

In some embodiments, the antibody is modified to increase its biological half-life. Various approaches are possible. For example, this may be done by increasing the binding affinity of the Fc region for FcRn. For example, one or more of the following residues can be mutated: 252, 254, 256, 433, 435, 436, as described in U.S. Pat. No. 6,277,375. Specific exemplary substitutions include one or more of the following: T252L, T254S, and/or T256F. Alternatively, to increase the biological half-life, the antibody can be altered within the CH1 or CL region to contain a salvage receptor binding epitope taken from two loops of a CH2 domain of an Fc region of an IgG, as described in U.S. Pat. Nos. 5,869,046 and 6,121,022 by Presta et al. Other exemplary variants that increase binding to FcRn and/or improve pharmacokinetic properties include substitutions at positions 259, 308, 428, and 434, including for example 259I, 308F, 428L, 428M, 434S, 434H, 434F, 434Y, and 434M. Other variants that increase Fc binding to FcRn include: 250E, 250Q, 428L, 428F, 250Q/428L (Hinton et al., 2004, J. Biol. Chem. 279(8): 6213-6216, Hinton et al. 2006 Journal of Immunology 176:346-356), 256A, 272A, 286A, 305A, 307A, 307Q, 311A, 312A, 376A, 378Q, 380A, 382A, 434A (Shields et al, Journal of Biological Chemistry, 2001, 276(9):6591-6604), 252F, 252T, 252Y, 252W, 254T, 256S, 256R, 256Q, 256E, 256D, 256T, 309P, 311S, 433R, 433S, 4331, 433P, 433Q, 434H, 434F, 434Y, 252Y/254T/256E, 433K/434F/436H, 308T/309P/311S (Dall Acqua et al. Journal of Immunology, 2002, 169:5171-5180, Dall'Acqua et al., 2006, Journal of Biological Chemistry 281:23514-23524). Other modifications for modulating FcRn binding are described in Yeung et al., 2010, J Immunol, 182:7663-7671. In some embodiments, hybrid IgG isotypes with particular biological characteristics may be used. For example, an IgG1/IgG3 hybrid variant may be constructed by substituting IgG 1 positions in the CH2 and/or CH3 region with the amino acids from IgG3 at positions where the two isotypes differ. Thus a hybrid variant IgG antibody may be constructed that comprises one or more substitutions, e.g., 274Q, 276K, 300F, 339T, 356E, 358M, 384S, 392N, 397M, 422I, 435R, and 436F. In other embodiments described herein, an IgG1/IgG2 hybrid variant may be constructed by substituting IgG2 positions in the CH2 and/or CH3 region with amino acids from IgG1 at positions where the two isotypes differ. Thus a hybrid variant IgG antibody may be constructed chat comprises one or more substitutions, e.g., one or more of the following amino acid substitutions: 233E, 234L, 235L, 236G (referring to an insertion of a glycine at position 236), and 321 h.

Moreover, the binding sites on human IgG1 for FcγRl, FcγRII, FcγRIII, and FcRn have been mapped, and variants with improved binding have been described (see Shields, R. L. et al. (2001) J. Biol. Chem. 276:6591-6604). Specific mutations at positions 256, 290, 298, 333, 334, and 339 were shown to improve binding to FcγRIII. Additionally, the following combination mutants were shown to improve FcγRIII binding: T256A/S298A, S298A/E333A, S298A/K224A, and S298A/E333A/K334A, which has been shown to exhibit enhanced FcγRIIIa binding and ADCC activity (Shields et al., 2001). Other IgG1 variants with strongly enhanced binding to FcγRIIIa have been identified, including variants with S239D/I332E and S239D/I332E/A330L mutations which showed the greatest increase in affinity for FcγRIIIa, a decrease in FcγRIIb binding, and strong cytotoxic activity in cynomolgus monkeys (Lazar et al., 2006). Introduction of the triple mutations into antibodies such as alemtuzumab (CD52-specific), trastuzumab (HER2/neu-specific), rituximab (CD20-specific), and cetuximab (EGFR-specific) translated into greatly enhanced ADCC activity in vitro, and the S239D/I332E variant showed an enhanced capacity to deplete B cells in monkeys (Lazar et al., 2006). In addition, IgG1 mutants containing L235V, F243L, R292P, Y300L and P396L mutations which exhibited enhanced binding to FcγRIIIa and concomitantly enhanced ADCC activity in transgenic mice expressing human FcγRIIIa in models of B cell malignancies and breast cancer have been identified (Stavenhagen et al., 2007; Nordstrom et al., 2011). Other Fc mutants that may be used include: S298A/E333A/L334A, S239D/I332E, S239D/I332E/A330L, L235V/F243L/R292P/Y300L/P396L, and M428L/N434S.

In some embodiments, an Fc is chosen that has reduced binding to FcγRs. An exemplary Fc, e.g., IgG1 Fc, with reduced FcγR binding, comprises the following three amino acid substitutions: L234A, L235E, and G237A. In some embodiments, an Fc is chosen that has reduced complement fixation. An exemplary Fc, e.g., IgG1 Fc, with reduced complement fixation, has the following two amino acid substitutions: A330S and P331S. In some embodiments, an Fc is chosen that has essentially no effector function, i.e., it has reduced binding to FcγRs and reduced complement fixation. An exemplary Fc, e.g., IgG1 Fc, that is effectorless, comprises the following five mutations: L234A, L235E, G237A, A330S, and P331S. When using an IgG4 constant domain, it is usually preferable to include the substitution S228P, which mimics the hinge sequence in IgG1 and thereby stabilizes IgG4 molecules.

Multivalent Antibodies

In one embodiment, the antibodies of the invention may be monovalent or multivalent (e.g., bivalent, trivalent, etc.). As used herein, the term “valency” refers to the number of potential target binding sites associated with an antibody. Each target binding site specifically binds one target molecule or specific position or locus on a target molecule. When an antibody is monovalent, each binding site of the molecule will specifically bind to a single antigen position or epitope. When an antibody comprises more than one target binding site (multivalent), each target binding site may specifically bind the same or different molecules (e.g., may bind to different ligands or different antigens, or different epitopes or positions on the same antigen). See, for example, U.S.P.N. 2009/0130105. In each case, at least one of the binding sites will comprise an epitope, motif or domain associated with a DLL3 isoform.

In one embodiment, the antibodies are bispecific antibodies in which the two chains have different specificities, as described in Millstein et al., 1983, Nature, 305:537-539. Other embodiments include antibodies with additional specificities such as trispecific antibodies. Other more sophisticated compatible multispecific constructs and methods of their fabrication are set forth in U.S.P.N. 2009/0155255, as well as WO 94/04690; Suresh et al., 1986, Methods in Enzymology, 121:210; and WO96/27011.

As stated above, multivalent antibodies may immunospecifically bind to different epitopes of the desired target molecule or may immunospecifically bind to both the target molecule as well as a heterologous epitope, such as a heterologous polypeptide or solid support material. In some embodiments, the multivalent antibodies may include bispecific antibodies or trispecific antibodies. Bispecific antibodies also include cross-linked or “heteroconjugate” antibodies. For example, one of the antibodies in the heteroconjugate can be coupled to avidin, the other to biotin. Such antibodies have, for example, been proposed to target immune system cells to unwanted cells (U.S. Pat. No. 4,676,980) and for treatment of HIV infection (WO 91/00360, WO 92/200373, and EP 03089). Heteroconjugate antibodies may be made using any convenient cross-linking methods. Suitable cross-linking agents are well known in the art and are disclosed in U.S. Pat. No. 4,676,980, along with a number of cross-linking techniques.

In some embodiments, antibody variable domains with the desired binding specificities (antibody-antigen combining sites) are fused to immunoglobulin constant domain sequences, such as an immunoglobulin heavy chain constant domain comprising at least part of the hinge, CH2, and/or CH3 regions, using methods well known to those of ordinary skill in the art.

Antibody Derivatives

An antibody provided herein may be further modified to contain additional nonproteinaceous moieties that are known in the art and readily available. The moieties suitable for derivatization of the antibody include but are not limited to water-soluble polymers.

Non-limiting examples of water-soluble polymers include, but are not limited to, PEG, copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, polypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight and may be branched or unbranched. The number of polymers attached to the antibody may vary, and if more than one polymer is attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

In another embodiment, conjugates of an antibody and nonproteinaceous moiety that may be selectively heated by exposure to radiation are provided. In one embodiment, the nonproteinaceous moiety is a carbon nanotube (Kam et al., Proc. Natl. Acad. Sci. USA 102: 11600-11605 (2005)). The radiation may be of any wavelength and includes, but is not limited to, wavelengths that do not harm ordinary cells, but which heat the nonproteinaceous moiety to a temperature at which cells proximal to the antibody-nonproteinaceous moiety are killed.

Another modification of the antibodies described herein is pegylation. An antibody can be pegylated to, for example, increase the biological (e.g., serum) half-life of the antibody. To pegylate an antibody, the antibody, or fragment thereof, typically is reacted with PEG, such as a reactive ester or aldehyde derivative of PEG, under conditions in which one or more PEG groups become attached to the antibody or antibody fragment. Preferably, the pegylation is carried out via an acylation reaction or an alkylation reaction with a reactive PEG molecule (or an analogous reactive water-soluble polymer). As used herein, the term “polyethylene glycol” is intended to encompass any of the forms of PEG that have been used to derivatize other proteins, such as mono (CI-CIO) alkoxy- or aryloxy-polyethylene glycol or polyethylene glycol-maleimide. In some embodiments, the antibody to be pegylated is an aglycosylated antibody. Methods for pegylating proteins are known in the art and can be applied to the antibodies described herein. See, for example, EP 0 154 316 by Nishimura et al. and EP0401384 by Ishikawa et al.

The present invention also encompasses a human monoclonal antibody described herein conjugated to a therapeutic agent, a polymer, a detectable label or enzyme. In one embodiment, the therapeutic agent is a cytotoxic agent. In one embodiment, the polymer is PEG.

Nucleic Acids, Expression Cassettes, and Vectors

The present invention provides isolated nucleic acid segments that encode the polypeptides, peptide fragments, and coupled proteins of the invention. The nucleic acid segments of the invention also include segments that encode for the same amino acids due to the degeneracy of the genetic code. For example, the amino acid threonine is encoded by ACU, ACC, ACA, and ACG and is therefore degenerate. It is intended that the invention includes all variations of the polynucleotide segments that encode for the same amino acids. Such mutations are known in the art (Watson et al., Molecular Biology of the Gene, Benjamin Cummings 1987). Mutations also include alteration of a nucleic acid segment to encode for conservative amino acid changes, for example, the substitution of leucine for isoleucine and so forth. Such mutations are also known in the art. Thus, the genes and nucleotide sequences of the invention include both the naturally occurring sequences as well as mutant forms.

The nucleic acid segments of the invention may be contained within a vector. A vector may include, but is not limited to, any plasmid, phagemid, F-factor, virus, cosmid, or phage in a double- or single-stranded linear or circular form which may or may not be self transmissible or mobilizable. The vector can also transform a prokaryotic or eukaryotic host either by integration into the cellular genome or exist extra-chromosomally (e.g., autonomous replicating plasmid with an origin of replication).

The nucleic acid segment in the vector can be under the control of, and operably linked to, an appropriate promoter or other regulatory elements for transcription in vitro or in a host cell, such as a eukaryotic cell, or a microbe, e.g., bacteria. The vector may be a shuttle vector that functions in multiple hosts. The vector may also be a cloning vector that typically contains one or a small number of restriction endonuclease recognition sites at which foreign DNA sequences can be inserted in a determinable fashion. Such insertion can occur without loss of essential biological function of the cloning vector. A cloning vector may also contain a marker gene that is suitable for use in the identification and selection of cells transformed with the cloning vector. Examples of marker genes are tetracycline resistance or ampicillin resistance. Many cloning vectors are commercially available (Stratagene, New England Biolabs, Clonetech).

The nucleic acid segments of the invention may also be inserted into an expression vector. Typically an expression vector contains prokaryotic DNA elements coding for a bacterial replication origin and an antibiotic resistance gene to provide for the amplification and selection of the expression vector in a bacterial host; regulatory elements that control initiation of transcription such as a promoter; and DNA elements that control the processing of transcripts such as introns, or a transcription termination/polyadenylation sequence.

Methods to introduce nucleic acid segment into a vector are available in the art (Sambrook et al., Molecular Cloning: A Laboratory Manual, 3rd edition, Cold Spring Harbor Press, Cold Spring Harbor, N.Y. (2001)). Briefly, a vector into which a nucleic acid segment is to be inserted is treated with one or more restriction enzymes (restriction endonuclease) to produce a linearized vector having a blunt end, a “sticky” end with a 5′ or a 3′ overhang, or any combination of the above. The vector may also be treated with a restriction enzyme and subsequently treated with another modifying enzyme, such as a polymerase, an exonuclease, a phosphatase or a kinase, to create a linearized vector that has characteristics useful for ligation of a nucleic acid segment into the vector. The nucleic acid segment that is to be inserted into the vector is treated with one or more restriction enzymes to create a linearized segment having a blunt end, a “sticky” end with a 5′ or a 3′ overhang, or any combination of the above. The nucleic acid segment may also be treated with a restriction enzyme and subsequently treated with another DNA modifying enzyme. Such DNA modifying enzymes include, but are not limited to, polymerase, exonuclease, phosphatase or a kinase, to create a nucleic acid segment that has characteristics useful for ligation of a nucleic acid segment into the vector.

The treated vector and nucleic acid segment are then ligated together to form a construct containing a nucleic acid segment according to methods available in the art (Sambrook et al., Molecular Cloning: A Laboratory Manual, 3rd edition, Cold Spring Harbor Press, Cold Spring Harbor, N.Y. (2001)). For example, the treated nucleic acid fragment and the treated vector are combined in the presence of a suitable buffer and ligase. The mixture is then incubated under appropriate conditions to allow the ligase to ligate the nucleic acid fragment into the vector.

The disclosure also provides an expression cassette which contains a nucleic acid sequence capable of directing expression of a particular nucleic acid segment of the invention, either in vitro or in a host cell. Also, a nucleic acid segment of the invention may be inserted into the expression cassette such that an anti-sense message is produced. The expression cassette is an isolatable unit such that the expression cassette may be in linear form and functional for in vitro transcription and translation assays. The materials and procedures to conduct these assays are commercially available from Promega Corp. (Madison, Wis.). For example, an in vitro transcript may be produced by placing a nucleic acid sequence under the control of a T7 promoter and then using T7 RNA polymerase to produce an in vitro transcript. This transcript may then be translated in vitro through use of a rabbit reticulocyte lysate. Alternatively, the expression cassette can be incorporated into a vector allowing for replication and amplification of the expression cassette within a host cell or also in vitro transcription and translation of a nucleic acid segment.

Such an expression cassette may contain one or a plurality of restriction sites allowing for placement of the nucleic acid segment under the regulation of a regulatory sequence. The expression cassette can also contain a termination signal operably linked to the nucleic acid segment as well as regulatory sequences required for proper translation of the nucleic acid segment. The expression cassette containing the nucleic acid segment may be chimeric, meaning that at least one of its components is heterologous with respect to at least one of its other components. The expression cassette may also be one that is naturally occurring but has been obtained in a recombinant form useful for heterologous expression. Expression of the nucleic acid segment in the expression cassette may be under the control of a constitutive promoter or an inducible promoter, which initiates transcription only when the host cell is exposed to some particular external stimulus.

The expression cassette may include in the 5′-3′ direction of transcription, a transcriptional and translational initiation region, a nucleic acid segment, and a transcriptional and translational termination region functional in vivo and/or in vitro. The termination region may be native with the transcriptional initiation region, may be native with the nucleic acid segment, or may be derived from another source.

The regulatory sequence can be a polynucleotide sequence located upstream (5′ non-coding sequences), within, or downstream (3′ non-coding sequences) of a coding sequence, and which influences the transcription, RNA processing or stability, or translation of the associated coding sequence. Regulatory sequences can include, but are not limited to, enhancers, promoters, repressor binding sites, translation leader sequences, introns, and polyadenylation signal sequences. They may include natural and synthetic sequences as well as sequences, which may be a combination of synthetic and natural sequences. While regulatory sequences are not limited to promoters, some useful regulatory sequences include constitutive promoters, inducible promoters, regulated promoters, tissue-specific promoters, viral promoters, and synthetic promoters.

A promoter is a nucleotide sequence that controls the expression of the coding sequence by providing the recognition for RNA polymerase and other factors required for proper transcription. A promoter includes a minimal promoter, consisting only of all basal elements needed for transcription initiation, such as a TATA-box and/or initiator that is a short DNA sequence comprised of a TATA-box and other sequences that serve to specify the site of transcription initiation, to which regulatory elements are added for control of expression. A promoter may be derived entirely from a native gene, or be composed of different elements derived from different promoters found in nature, or even be comprised of synthetic DNA segments. A promoter may contain DNA sequences that are involved in the binding of protein factors that control the effectiveness of transcription initiation in response to physiological or developmental conditions.

The disclosure also provides a construct containing a vector and an expression cassette. The vector may be selected from, but not limited to, any vector previously described. Into this vector may be inserted an expression cassette through methods known in the art and previously described (Sambrook et al., Molecular Cloning: A Laboratory Manual, 3rd edition, Cold Spring Harbor Press, Cold Spring Harbor, N.Y. (2001)). In one embodiment, the regulatory sequences of the expression cassette may be derived from a source other than the vector into which the expression cassette is inserted. In another embodiment, a construct containing a vector and an expression cassette is formed upon insertion of a nucleic acid segment of the invention into a vector that itself contains regulatory sequences. Thus, an expression cassette is formed upon insertion of the nucleic acid segment into the vector. Vectors containing regulatory sequences are available commercially, and methods for their use are known in the art (Clonetech, Promega, Stratagene).

In another aspect, this disclosure also provides (i) a nucleic acid molecule encoding a polypeptide chain of the antibody or antigen-binding fragment thereof described above; (ii) a vector comprising the nucleic acid molecule as described; and (iii) a cultured host cell comprising the vector as described.

Also provided is a method for producing a polypeptide (e.g., anti-TBEV antibody), comprising: (a) obtaining the cultured host cell as described; (b) culturing the cultured host cell in a medium under conditions permitting expression of a polypeptide encoded by the vector and assembling of an antibody or fragment thereof, and (c) purifying the antibody or fragment from the cultured cell or the medium of the cell.

Methods of Production

Antibodies may be produced using recombinant methods and compositions, e.g., as described in U.S. Pat. No. 4,816,567. In one embodiment, an isolated nucleic acid encoding an antibody described herein is provided. Such nucleic acid may encode an amino acid sequence comprising the VL and/or an amino acid sequence comprising the VH of the antibody (e.g., the light and/or heavy chains of the antibody). In a further embodiment, one or more vectors (e.g., expression vectors) comprising such nucleic acid are provided. In a further embodiment, a host cell comprising such nucleic acid is provided. In one such embodiment, a host cell comprises (e.g., has been transformed with): (1) a vector comprising a nucleic acid that encodes an amino acid sequence comprising the VL of the antibody and an amino acid sequence comprising the VH of the antibody, or (2) a first vector comprising a nucleic acid that encodes an amino acid sequence comprising the VL of the antibody and a second vector comprising a nucleic acid that encodes an amino acid sequence comprising the VH of the antibody. In one embodiment, the host cell is eukaryotic, e.g., a Chinese Hamster Ovary (CHO) cell or lymphoid cell (e.g., Y0, NS0, Sp20 cell). In one embodiment, a method of making an antibody is provided, wherein the method comprises culturing a host cell comprising a nucleic acid encoding the antibody, as provided above, under conditions suitable for expression of the antibody, and optionally recovering the antibody from the host cell (or host cell culture medium).

For recombinant production of an antibody, a nucleic acid encoding an antibody, e.g., as described above, is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

A described herein, such as in the examples, RNA encoding the disclosed antibodies can be isolated from convalescent or vaccinated donors and reverse transcribed into cDNA. The cDNA can then be modified through recombinant DNA methods and cloned into one or more vectors for expression in a host cell. The recombinantly expressed monoclonal antibodies can be isolated and subject to further purification. At least due to one or more of the reverse transcription, PCR amplification, and cloning processes, each of the recombinantly made and expressed antibodies possesses at least one or more non-naturally occurring changes or mutations in the heavy chain variable region, light chain variable region, or constant region, distinguishing it from an occurring natural antibody. These mutations render the antibodies disclosed herein markedly different from any naturally occurring counterparts. Accordingly, the antibodies disclosed herein are non-naturally occurring.

Suitable host cells for cloning or expression of antibody-encoding vectors include prokaryotic or eukaryotic cells described herein. For example, antibodies may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, Methods in Molecular Biology, Vol. 248 (B. K. C. Lo, ed., Humana Press, Totowa, N.J., 2003), pp. 245-254, describing expression of antibody fragments in E. coli.) After expression, the antibody may be isolated from the bacterial cell paste in a soluble fraction and can be further purified.

In addition to prokaryotes, eukaryotic microbes such as filamentous fungi or yeast are suitable cloning or expression hosts for antibody-encoding vectors, including fungi and yeast strains whose glycosylation pathways have been “humanized,” resulting in the production of an antibody with a partially or fully human glycosylation pattern. See Gerngross, Nat. Biotech. 22:1409-1414 (2004), and Li et al., Nat. Biotech. 24:210-215 (2006).

Suitable host cells for the expression of glycosylated antibody are also derived from multicellular organisms (invertebrates and vertebrates). Examples of invertebrate cells include plant and insect cells. Numerous baculoviral strains have been identified, which may be used in conjunction with insect cells, particularly for transfection of Spodoptera frugiperda cells.

Plant cell cultures can also be utilized as hosts. See, e.g., U.S. Pat. Nos. 5,959,177, 6,040,498, 6,420,548, 7,125,978, and 6,417,429 (describing PLANTIBODIES technology for producing antibodies in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather et al., Annals N.Y. Acad. Sci. 383:44-68 (1982); MRC 5 cells; and FS4 cells. Other useful mammalian host cell lines include CHO cells, including DHFR-CHO cells (Urlaub et al., Proc. Natl. Acad. Sci. USA 77:4216 (1980)); and myeloma cell lines such as Y0, NS0, and Sp2/0. For a review of certain mammalian host cell lines suitable for antibody production, see, e.g., Yazaki and Wu, Methods in Molecular Biology, Vol. 248 (B. K. C. Lo, ed., Humana Press, Totowa, N.J.), pp. 255-268 (2003).

### B. Compositions and Formulations

The antibodies of this invention represent an excellent way for the development of antiviral therapies either alone or in antibody cocktails with additional anti-TBEV antibodies for the treatment of tick-borne flavivirus (e.g., TBEV) infection in humans.

In another aspect, the present invention provides a pharmaceutical composition comprising the antibodies of the present invention described herein formulated together with a pharmaceutically acceptable carrier. The composition may optionally contain one or more additional pharmaceutically active ingredients, such as another antibody or a therapeutic agent.

In some embodiments, the pharmaceutical comprises two or more of the antibody or antigen-binding fragment thereof described above, such as any combinations of the antibody or antigen-binding fragment thereof comprising a heavy chain and a light chain that comprise the respective amino acid sequences described herein.

In some example, each antibody or antigen-binding fragment thereof comprises (i) HCDRs1-3 and LCDRs1-3 of an antibody selected from those in Tables 2A-I, 3, and 4, or (ii) a heavy chain variable region and a light chain variable region that comprise the respective amino acid sequences of an antibody selected from those in Tables 2A-I, 3, and 4.

The pharmaceutical compositions of the invention also can be administered in a combination therapy with, for example, another immune-stimulatory agent, an antiviral agent, or a vaccine, etc. In some embodiments, a composition comprises an antibody of this invention at a concentration of at least 1 mg/ml, 5 mg/ml, 10 mg/ml, 50 mg/ml, 100 mg/ml, 150 mg/ml, 200 mg/ml, 1-300 mg/ml, or 100-300 mg/ml.

In some embodiments, the second therapeutic agent comprises an anti-inflammatory drug or an antiviral compound. In some embodiments, the antiviral compound comprises: a nucleoside analog, a peptoid, an oligopeptide, a polypeptide, a protease inhibitor, a 3C-like protease inhibitor, a papain-like protease inhibitor, or an inhibitor of an RNA dependent RNA polymerase. In some embodiments, the antiviral compound may include: acyclovir, gancyclovir, vidarabine, foscarnet, cidofovir, amantadine, ribavirin, trifluorothymidine, zidovudine, didanosine, zalcitabine or an interferon. In some embodiments, the interferon is an interferon-α or an interferon-β.

Also within the scope of this disclosure is use of the pharmaceutical composition in the preparation of a medicament for the diagnosis, prophylaxis, treatment, or combination thereof of a condition resulting from infection caused by a tick-borne flavivirus (e.g., TBEV).

The pharmaceutical composition can comprise any number of excipients. Excipients that can be used include carriers, surface-active agents, thickening or emulsifying agents, solid binders, dispersion or suspension aids, solubilizers, colorants, flavoring agents, coatings, disintegrating agents, lubricants, sweeteners, preservatives, isotonic agents, and combinations thereof. The selection and use of suitable excipients is taught in Gennaro, ed., Remington: The Science and Practice of Pharmacy, 20th Ed. (Lippincott Williams & Wilkins 2003), the disclosure of which is incorporated herein by reference.

Preferably, a pharmaceutical composition is suitable for intravenous, intramuscular, subcutaneous, parenteral, spinal or epidermal administration (e.g., by injection or infusion). Depending on the route of administration, the active compound can be coated in a material to protect it from the action of acids and other natural conditions that may inactivate it. The phrase “parenteral administration” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion. Alternatively, an antibody of the present invention described herein can be administered via a non-parenteral route, such as a topical, epidermal or mucosal route of administration, e.g., intranasally, orally, vaginally, rectally, sublingually or topically.

The pharmaceutical compositions of the invention may be prepared in many forms that include tablets, hard or soft gelatin capsules, aqueous solutions, suspensions, and liposomes and other slow-release formulations, such as shaped polymeric gels. An oral dosage form may be formulated such that the antibody is released into the intestine after passing through the stomach. Such formulations are described in U.S. Pat. No. 6,306,434 and in the references contained therein.

Oral liquid pharmaceutical compositions may be in the form of, for example, aqueous or oily suspensions, solutions, emulsions, syrups or elixirs, or may be presented as a dry product for constitution with water or other suitable vehicle before use. Such liquid pharmaceutical compositions may contain conventional additives such as suspending agents, emulsifying agents, non-aqueous vehicles (which may include edible oils), or preservatives.

An antibody can be formulated for parenteral administration (e.g., by injection, for example, bolus injection or continuous infusion) and may be presented in unit dosage form in ampules, prefilled syringes, small volume infusion containers or multi-dose containers with an added preservative. The pharmaceutical compositions may take such forms as suspensions, solutions, or emulsions in oily or aqueous vehicles and may contain formulatory agents such as suspending, stabilizing and/or dispersing agents. Pharmaceutical compositions suitable for rectal administration can be prepared as unit dose suppositories. Suitable carriers include saline solution and other materials commonly used in the art.

For administration by inhalation, an antibody can be conveniently delivered from an insufflator, nebulizer or a pressurized pack or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount.

Alternatively, for administration by inhalation or insufflation, an antibody may take the form of a dry powder composition, for example, a powder mix of a modulator and a suitable powder base such as lactose or starch. The powder composition may be presented in a unit dosage form in, for example, capsules or cartridges or, e.g., gelatin or blister packs from which the powder may be administered with the aid of an inhalator or insufflator. For intra-nasal administration, an antibody may be administered via a liquid spray, such as via a plastic bottle atomizer.

Pharmaceutical compositions of the invention may also contain other ingredients such as flavorings, colorings, anti-microbial agents, or preservatives. It will be appreciated that the amount of an antibody required for use in treatment will vary not only with the particular carrier selected but also with the route of administration, the nature of the condition being treated, and the age and condition of the patient. Ultimately the attendant health care provider may determine proper dosage. In addition, a pharmaceutical composition may be formulated as a single unit dosage form.

The pharmaceutical composition of the present invention can be in the form of sterile aqueous solutions or dispersions. It can also be formulated in a microemulsion, liposome, or other ordered structure suitable to high drug concentration.

An antibody of the present invention described herein can be administered as a sustained release formulation, in which case less frequent administration is required. Dosage and frequency vary depending on the half-life of the antibody in the patient. In general, human antibodies show the longest half-life, followed by humanized antibodies, chimeric antibodies, and nonhuman antibodies. The dosage and frequency of administration can vary depending on whether the treatment is prophylactic or therapeutic. In prophylactic applications, a relatively low dosage is administered at relatively infrequent intervals over a long period of time. Some patients continue to receive treatment for the rest of their lives. In therapeutic applications, a relatively high dosage at relatively short intervals is sometimes required until progression of the disease is reduced or terminated, and preferably, until the patient shows partial or complete amelioration of symptoms of the disease. Thereafter, the patient can be administered a prophylactic regime.

The amount of active ingredient that can be combined with a carrier material to produce a single dosage form will vary depending upon the subject being treated and the particular mode of administration and will generally be that amount of the composition, which produces a therapeutic effect. Generally, out of one hundred percent, this amount will range from about 0.01% to about 99% of active ingredient, preferably from about 0.1% to about 70%, most preferably from about 1% to about 30% of active ingredient in combination with a pharmaceutically acceptable carrier.

Dosage regimens can be adjusted to provide the optimum desired response (e.g., a therapeutic response). For example, a single bolus can be administered, several divided doses can be administered over time or the dose can be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. Alternatively, the antibody can be administered as a sustained release formulation, in which case less frequent administration is required. For administration of the antibody, the dosage ranges from about 0.0001 to 800 mg/kg, and more usually 0.01 to 5 mg/kg, of the host body weight. For example dosages can be 0.3 mg/kg body weight, 1 mg/kg body weight, 3 mg/kg body weight, 5 mg/kg body weight or 10 mg/kg body weight or within the range of 1-10 mg/kg. An exemplary treatment regime entails administration once per week, once every two weeks, once every three weeks, once every four weeks, once a month, once every 3 months or once every three to 6 months. Preferred dosage regimens for an antibody of the invention include 1 mg/kg body weight or 3 mg/kg body weight via intravenous administration, with the antibody being given using one of the following dosing schedules: (i) every four weeks for six dosages, then every three months; (ii) every three weeks; (iii) 3 mg/kg body weight once followed by 1 mg/kg body weight every three weeks. In some methods, dosage is adjusted to achieve a plasma antibody concentration of about 1-1000 μg/ml and in some methods about 25-300 μg/ml. A “therapeutically effective dosage” of an antibody of the invention preferably results in a decrease in severity of disease symptoms, an increase in frequency and duration of disease symptom-free periods, or a prevention of impairment or disability due to the disease affliction. For example, for the treatment of tick-borne flavivirus (e.g., TBEV) infection in a subject, a “therapeutically effective dosage” preferably inhibits a tick-borne flavivirus (e.g., TBEV) replication or uptake by host cells by at least about 20%, more preferably by at least about 40%, even more preferably by at least about 60%, and still more preferably by at least about 80% relative to untreated subjects. A therapeutically effective amount of a therapeutic compound can neutralize a tick-borne flavivirus (e.g., TBEV), or otherwise ameliorate symptoms in a subject, which is typically a human or can be another mammal.

The pharmaceutical composition can be a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. See, e.g., Sustained and Controlled Release Drug Delivery Systems, J. R. Robinson, ed., Marcel Dekker, Inc., New York, 1978.

Therapeutic compositions can be administered via medical devices such as (1) needleless hypodermic injection devices (e.g., U.S. Pat. Nos. 5,399,163; 5,383,851; 5,312,335; 5,064,413; 4,941,880; 4,790,824; and 4,596,556); (2) micro-infusion pumps (U.S. Pat. No. 4,487,603); (3) transdermal devices (U.S. Pat. No. 4,486,194); (4) infusion apparati (U.S. Pat. Nos. 4,447,233 and 4,447,224); and (5) osmotic devices (U.S. Pat. Nos. 4,439,196 and 4,475,196); the disclosures of which are incorporated herein by reference.

In some embodiments, the human monoclonal antibodies described herein can be formulated to ensure proper distribution in vivo. For example, to ensure that the therapeutic compounds of the invention cross the blood-brain barrier, they can be formulated in liposomes, which may additionally comprise targeting moieties to enhance selective transport to specific cells or organs. See, e.g., U.S. Pat. Nos. 4,522,811; 5,374,548; 5,416,016; and 5,399,331; V. V. Ranade (1989) Clin. Pharmacol. 29:685; Umezawa et al., (1988) Biochem. Biophys. Res. Commun. 153:1038; Bloeman et al. (1995) FEBS Lett. 357:140; M. Owais et al. (1995) Antimicrob. Agents Chemother. 39:180; Briscoe et al. (1995) Am. Physiol. 1233:134; Schreier et al. (1994). Biol. Chem. 269:9090; Keinanen and Laukkanen (1994) FEBS Lett. 346:123; and Killion and Fidler (1994) Immunomethods 4:273.

In some embodiments, the initial dose may be followed by administration of a second or a plurality of subsequent doses of the antibody or antigen-binding fragment thereof in an amount that can be approximately the same or less than that of the initial dose, wherein the subsequent doses are separated by at least 1 day to 3 days; at least one week, at least 2 weeks; at least 3 weeks; at least 4 weeks; at least 5 weeks; at least 6 weeks; at least 7 weeks; at least 8 weeks; at least 9 weeks; at least 10 weeks; at least 12 weeks; or at least 14 weeks.

Various delivery systems are known and can be used to administer the pharmaceutical composition of the invention, e.g., encapsulation in liposomes, microparticles, microcapsules, recombinant cells capable of expressing the mutant viruses, receptor-mediated endocytosis (see, e.g., Wu et al. (1987) J. Biol. Chem. 262:4429-4432). Methods of introduction include, but are not limited to, intradermal, transdermal, intramuscular, intraperitoneal, intravenous, subcutaneous, intranasal, epidural, and oral routes. The composition may be administered by any convenient route, for example, by infusion or bolus injection, by absorption through epithelial or mucocutaneous linings (e.g., oral mucosa, rectal and intestinal mucosa, etc.) and may be administered together with other biologically active agents. Administration can be systemic or local. The pharmaceutical composition can also be delivered in a vesicle, in particular, a liposome (see, for example, Langer (1990) Science 249: 1527-1533).

The use of nanoparticles to deliver the antibodies of the present invention is also contemplated herein. Antibody-conjugated nanoparticles may be used both for therapeutic and diagnostic applications. Antibody-conjugated nanoparticles and methods of preparation and use are described in detail by Arruebo, M., et al. 2009 (“Antibody-conjugated nanoparticles for biomedical applications” in J. Nanomat. Volume 2009, Article ID 439389), incorporated herein by reference. Nanoparticles may be developed and conjugated to antibodies contained in pharmaceutical compositions to target cells. Nanoparticles for drug delivery have also been described in, for example, U.S. Pat. No. 8,257,740, or U.S. Pat. No. 8,246,995, each incorporated herein in its entirety.

In certain situations, the pharmaceutical composition can be delivered in a controlled release system. In one embodiment, a pump may be used. In another embodiment, polymeric materials can be used. In yet another embodiment, a controlled release system can be placed in proximity of the composition's target, thus requiring only a fraction of the systemic dose.

The injectable preparations may include dosage forms for intravenous, subcutaneous, intracutaneous, intracranial, intraperitoneal, and intramuscular injections, drip infusions, etc. These injectable preparations may be prepared by methods publicly known. For example, the injectable preparations may be prepared, e.g., by dissolving, suspending or emulsifying the antibody or its salt described above in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant [e.g., polysorbate 80, HCO-50 (polyoxyethylene (50 mol) adduct of hydrogenated castor oil)], etc. As the oily medium, there are employed, e.g., sesame oil, soybean oil, etc., which may be used in combination with a solubilizing agent such as benzyl benzoate, benzyl alcohol, etc. The injection thus prepared is preferably filled in an appropriate ampoule.

A pharmaceutical composition of the present invention can be delivered subcutaneously or intravenously with a standard needle and syringe. In addition, with respect to subcutaneous delivery, a pen delivery device readily has applications in delivering a pharmaceutical composition of the present invention. Such a pen delivery device can be reusable or disposable. A reusable pen delivery device generally utilizes a replaceable cartridge that contains a pharmaceutical composition. Once all of the pharmaceutical composition within the cartridge has been administered and the cartridge is empty, the empty cartridge can readily be discarded and replaced with a new cartridge that contains the pharmaceutical composition. The pen delivery device can then be reused. In a disposable pen delivery device, there is no replaceable cartridge. Rather, the disposable pen delivery device comes prefilled with the pharmaceutical composition held in a reservoir within the device. Once the reservoir is emptied of the pharmaceutical composition, the entire device is discarded.

Numerous reusable pen and autoinjector delivery devices have applications in the subcutaneous delivery of a pharmaceutical composition of the present invention. Examples include, but certainly are not limited to AUTOPEN™ (Owen Mumford, Inc., Woodstock, UK), DISETRONIC™ pen (Disetronic Medical Systems, Burghdorf, Switzerland), HUMALOG MIX 75/25™ pen, HUMALOG™ pen, HUMALIN 70/30™ pen (Eli Lilly and Co., Indianapolis, IN), NOVOPEN™ I, II and III (Novo Nordisk, Copenhagen, Denmark), NOVOPEN JUNIOR™ (Novo Nordisk, Copenhagen, Denmark), BD™ pen (Becton Dickinson, Franklin Lakes, NJ), OPTIPEN™ OPTIPEN PRO™, OPTIPEN STARLET™, and OPTICLIK™ (Sanofi-Aventis, Frankfurt, Germany), to name only a few. Examples of disposable pen delivery devices having applications in subcutaneous delivery of a pharmaceutical composition of the present invention include, but certainly are not limited to the SOLOSTAR™ pen (Sanofi-Aventis), the FLEXPEN™ (Novo Nordisk), and the KWIKPEN™ (Eli Lilly), the SURECLICK™ Autoinjector (Amgen, Thousand Oaks, CA), the PENLET™ (Haselmeier, Stuttgart, Germany), the EPIPEN (Dey, L.P.) and the HUMIRA™ Pen (Abbott Labs, Abbott Park, IL), to name only a few.

Advantageously, the pharmaceutical compositions for oral or parenteral use described above are prepared into dosage forms in a unit dose suited to fit a dose of the active ingredients. Such dosage forms in a unit dose include, for example, tablets, pills, capsules, injections (ampoules), suppositories, etc. The amount of the antibody contained is generally about 5 to about 500 mg per dosage form in a unit dose; especially in the form of injection, it is preferred that the antibody is contained in about 5 to about 300 mg and in about 10 to about 300 mg for the other dosage forms.

### C. Methods of Use

Methods of Treatment

The antibodies, compositions, and formulations described herein can be used to neutralize a tick-borne flavivirus (e.g., TBEV) and thereby treating or preventing diseases or infections caused by various tick-borne flaviviruses, including TBEV.

Accordingly, in one aspect, this disclosure further provides a method of neutralizing a tick-borne flavivirus (e.g., TBEV) in a subject. The method comprises administering to a subject in need thereof a therapeutically effective amount of the antibody or antigen-binding fragment thereof or a therapeutically effective amount of the pharmaceutical composition, as described above.

In another aspect, this disclosure additionally provides a method of preventing or treating tick-borne flavivirus (e.g., TBEV) infection. The method comprises administering to a subject in need thereof a therapeutically effective amount of the antibody or antigen-binding fragment thereof or a therapeutically effective amount of the pharmaceutical composition, as described above.

For example, the neutralizing of a TBEV can be carried out via (i) inhibiting TBEV binding to a target cell; (ii) inhibiting TBEV uptake by a target cell; (iii) inhibiting TBEV replication; and (iv) inhibiting TBEV virus particle release from infected cells. One skilled in the art possesses the ability to perform any assay to assess neutralization of TBEV.

Notably, the neutralizing properties of antibodies may be assessed by a variety of tests, which all may assess the consequences of (i) inhibition of TBEV binding to a target cell; (ii) inhibition of TBEV uptake by a target cell; (iii) inhibition of TBEV replication; and (iv) inhibition of TBEV virus particle release from infected cells. In other words, implementing different tests may lead to the observation of the same consequence, i.e., the loss of infectivity of the TBEV. Thus, in one embodiment, the present invention provides a method of neutralizing TBEV in a subject comprising administering to the subject a therapeutically effective amount of the antibody of the present invention described herein.

Another aspect of the present invention provides a method of treating a tick-borne flavivirus (e.g., TBEV)-related disease. Such a method includes therapeutic (e.g., following TBEV infection) and prophylactic (e.g., prior to TBEV exposure, infection or pathology). For example, therapeutic and prophylactic methods of treating an individual for TBEV infection include treatment of an individual having or at risk of having TBEV infection or pathology, treating an individual with a TBEV infection, and methods of protecting an individual from TBEV infection, to decrease or reduce the probability of TBEV infection in an individual, to decrease or reduce susceptibility of an individual to TBEV infection, or to inhibit or prevent TBEV infection in an individual, and to decrease, reduce, inhibit or suppress transmission of a TBEV from an infected individual to an uninfected individual. Such methods include administering an antibody of the present invention or a composition comprising the antibody disclosed herein to therapeutically or prophylactically treat (vaccinate or immunize) an individual having or at risk of having TBEV infection or pathology. Accordingly, methods can treat the TBEV infection or pathology, or provide the individual with protection from infection (e.g., prophylactic protection).

In one embodiment, a method of treating a tick-borne flavivirus (e.g., TBEV)-related disease comprises administering to an individual in need thereof an antibody or therapeutic composition disclosed herein in an amount sufficient to reduce one or more physiological conditions or symptoms associated with tick-borne flavivirus (e.g., TBEV) infection or pathology, thereby treating the tick-borne flavivirus (e.g., TBEV)-related disease.

In one embodiment, an antibody or therapeutic composition disclosed herein is used to treat a tick-borne flavivirus (e.g., TBEV)-related disease. In some embodiments, use of an antibody or therapeutic composition disclosed herein treats a TBEV-related disease by reducing one or more physiological conditions or symptoms associated with TBEV infection or pathology. In aspects of this embodiment, administration of an antibody or therapeutic composition disclosed herein is in an amount sufficient to reduce one or more physiological conditions or symptoms associated with TBEV infection or pathology, thereby treating the TBEV-based disease. In other aspects of this embodiment, administration of an antibody or therapeutic composition disclosed herein is in an amount sufficient to increase, induce, enhance, augment, promote or stimulate TBEV clearance or removal; or decrease, reduce, inhibit, suppress, prevent, control, or limit transmission of TBEV to another individual.

One or more physiological conditions or symptoms associated with tick-borne flavivirus (e.g., TBEV) infection or pathology will respond to a method of treatment disclosed herein. The symptoms of tick-borne flavivirus (e.g., TBEV) infection or pathology vary, depending on the phase of infection.

In some embodiments, the method of neutralizing a tick-borne flavivirus (e.g., TBEV) in a subject comprises administering to a subject in need thereof a therapeutically effective amount of a first antibody or antigen-binding fragment thereof and a second antibody or antigen-binding fragment thereof of the antibody or antigen-binding fragment, as described above, wherein the first antibody or antigen-binding fragment thereof and the second antibody or antigen binding fragment thereof exhibit synergistic activity or a therapeutically effective amount of the pharmaceutical composition described above.

In some embodiments, the method of preventing or treating tick-borne flavivirus (e.g., TBEV) infection comprises administering to a subject in need thereof a therapeutically effective amount of a first antibody or antigen-binding fragment thereof and a second antibody or antigen-binding fragment thereof of the antibody or antigen-binding fragment, as described above, wherein the first antibody or antigen-binding fragment thereof and the second antibody or antigen binding fragment thereof exhibit synergistic activity or a therapeutically effective amount of the pharmaceutical composition described above. In some embodiments, the first antibody or antigen-binding fragment thereof is administered before, after, or concurrently with the second antibody or antigen-binding fragment thereof.

In some embodiments, the first antibody or antigen-binding fragment thereof and the second antibody or antigen-binding fragment thereof can be any combinations of the antibody or antigen-binding fragment thereof comprising a heavy chain and a light chain that comprise the respective amino acid sequences described herein.

In some embodiments, the second therapeutic agent comprises an anti-inflammatory drug or an antiviral compound. In some embodiments, the antiviral compound comprises: a nucleoside analog, a peptoid, an oligopeptide, a polypeptide, a protease inhibitor, a 3C-like protease inhibitor, a papain-like protease inhibitor, or an inhibitor of an RNA dependent RNA polymerase. In some embodiments, the antiviral compound may include: acyclovir, gancyclovir, vidarabine, foscarnet, cidofovir, amantadine, ribavirin, trifluorothymidine, zidovudine, didanosine, zalcitabine or an interferon. In some embodiments, the interferon is an interferon-α or an interferon-β.

In some embodiments, the antibody or antigen-binding fragment thereof is administered before, after, or concurrently with the second therapeutic agent or therapy. In some embodiments, the antibody or antigen-binding fragment thereof is administered to the subject intravenously, subcutaneously, or intraperitoneally. In some embodiments, the antibody or antigen-binding fragment thereof is administered prophylactically or therapeutically.

The antibodies described herein can be used together with one or more of other anti-TBEV virus antibodies to neutralize a tick-borne flavivirus (e.g., TBEV) and thereby treating tick-borne flavivirus (e.g., TBEV) infection.

Combination Therapies

Combination therapies may include an anti-TBEV antibody as described and any additional therapeutic agent that may be advantageously combined with an antibody or a biologically active fragment of an antibody as described. The antibodies may be combined synergistically with one or more drugs or therapy used to treat a disease or disorder associated with a viral infection, such as tick-borne flavivirus (e.g., TBEV) infection. In some embodiments, the antibodies of the invention may be combined with a second therapeutic agent to ameliorate one or more symptoms of said disease. In some embodiments, the antibodies may be combined with a second antibody to provide synergistic activity in ameliorating one or more symptoms of said disease. In some embodiments, the first antibody or antigen-binding fragment thereof is administered before, after, or concurrently with the second antibody or antigen-binding fragment thereof.

For example, the antibody described herein can be used in various detection methods for use in, e.g., monitoring the progression of tick-borne flavivirus (e.g., TBEV) infection; monitoring patient response to treatment for such an infection, etc.

In some embodiments, the second therapeutic agent is another antibody to a tick-borne flavivirus (e.g., TBEV) protein or a fragment thereof. It is contemplated herein to use a combination (“cocktail”) of antibodies with broad neutralization or inhibitory activity against a tick-borne flavivirus (e.g., TBEV). In some embodiments, non-competing antibodies may be combined and administered to a subject in need thereof. In some embodiments, the antibodies comprising the combination bind to distinct non-overlapping epitopes on the protein. In some embodiments, the second antibody may possess a longer half-life in human serum.

As used herein, the term “in combination with” means that additional therapeutically active component(s) may be administered prior to, concurrent with, or after the administration of the anti-TBEV antibody of the present invention. The term “in combination with” also includes sequential or concomitant administration of an anti-TBEV antibody and a second therapeutic agent.

The additional therapeutically active component(s) may be administered to a subject prior to administration of an anti-TBEV antibody of the present invention. For example, a first component may be deemed to be administered “prior to” a second component if the first component is administered 1 week before, 72 hours before, 60 hours before, 48 hours before, 36 hours before, 24 hours before, 12 hours before, 6 hours before, 5 hours before, 4 hours before, 3 hours before, 2 hours before, 1 hour before, 30 minutes before, 15 minutes before, 10 minutes before, 5 minutes before, or less than 1 minute before administration of the second component. In other embodiments, the additional therapeutically active component(s) may be administered to a subject after administration of an anti-TBEV antibody of the present invention. For example, a first component may be deemed to be administered “after” a second component if the first component is administered 1 minute after, 5 minutes after, 10 minutes after, 15 minutes after, 30 minutes after, 1 hour after, 2 hours after, 3 hours after, 4 hours after, 5 hours after, 6 hours after, 12 hours after, 24 hours after, 36 hours after, 48 hours after, 60 hours after, 72 hours after administration of the second component. In yet other embodiments, the additional therapeutically active component(s) may be administered to a subject concurrent with administration of an anti-TBEV antibody of the present invention. “Concurrent” administration, for purposes of the present invention, includes, e.g., administration of an anti-TBEV antibody and an additional therapeutically active component to a subject in a single dosage form or in separate dosage forms administered to the subject within about 30 minutes or less of each other. If administered in separate dosage forms, each dosage form may be administered via the same route (e.g., both the anti-TBEV antibody and the additional therapeutically active component may be administered intravenously, etc.); alternatively, each dosage form may be administered via a different route (e.g., the anti-TBEV antibody may be administered intravenously, and the additional therapeutically active component may be administered orally). In any event, administering the components in a single dosage from, in separate dosage forms by the same route, or in separate dosage forms by different routes are all considered “concurrent administration,” for purposes of the present disclosure. For purposes of the present disclosure, administration of an anti-TBEV antibody “prior to,” “concurrent with,” or “after” (as those terms are defined hereinabove) administration of an additional therapeutically active component is considered administration of an anti-TBEV antibody “in combination with” an additional therapeutically active component.

The present invention includes pharmaceutical compositions in which an anti-TBEV antibody of the present invention is co-formulated with one or more of the additional therapeutically active component(s) as described elsewhere herein.

Administration Regimens

According to certain embodiments, a single dose of an anti-TBEV antibody as described (or a pharmaceutical composition comprising a combination of an anti-TBEV antibody and any of the additional therapeutically active agents mentioned herein) may be administered to a subject in need thereof. According to certain embodiments of the present invention, multiple doses of an anti-TBEV antibody (or a pharmaceutical composition comprising a combination of an anti-TBEV antibody and any of the additional therapeutically active agents mentioned herein) may be administered to a subject over a defined time course. The methods according to this aspect of the invention comprise sequentially administering to a subject multiple doses of an anti-TBEV antibody. As used herein, “sequentially administering” means that each dose of anti-TBEV antibody is administered to the subject at a different point in time, e.g., on different days separated by a predetermined interval (e.g., hours, days, weeks or months). This disclosure provides methods which comprise sequentially administering to the patient a single initial dose of an anti-TBEV antibody, followed by one or more secondary doses of the anti-TBEV antibody, and optionally followed by one or more tertiary doses of the anti-TBEV antibody.

The terms “initial dose,” “secondary doses,” and “tertiary doses,” refer to the temporal sequence of administration of the anti-TBEV antibody of the invention. Thus, the “initial dose” is the dose which is administered at the beginning of the treatment regimen (also referred to as the “baseline dose”); the “secondary doses” are the doses which are administered after the initial dose; and the “tertiary doses” are the doses which are administered after the secondary doses. The initial, secondary, and tertiary doses may all contain the same amount of anti-TBEV antibody, but generally may differ from one another in terms of frequency of administration. In some embodiments, however, the amount of anti-TBEV antibody contained in the initial, secondary and/or tertiary doses varies from one another (e.g., adjusted up or down as appropriate) during the course of treatment. In some embodiments, two or more (e.g., 2, 3, 4, or 5) doses are administered at the beginning of the treatment regimen as “loading doses” followed by subsequent doses that are administered on a less frequent basis (e.g., “maintenance doses”).

In certain exemplary embodiments of the present invention, each secondary and/or tertiary dose is administered 1 to 48 hours (e.g., 1, 1½, 2, 2½, 3, 3½, 4, 4½, 5, 5½, 6, 6½, 7, 7½, 8, 8½, 9, 9½, 10, 10½, 11, 11½, 12, 12½, 13, 13½, 14, 14½, 15, 15½, 16, 16½, 17, 17½, 18, 18½, 19, 19½, 20, 20½, 21, 21½, 22, 22½, 23, 23½24, 24½, 25, 25½26, 26½, or more) after the immediately preceding dose. The phrase “the immediately preceding dose,” as used herein, means, in a sequence of multiple administrations, the dose of anti-TBEV antibody, which is administered to a patient prior to the administration of the very next dose in the sequence with no intervening doses.

The methods, according to this aspect of the invention, may comprise administering to a patient any number of secondary and/or tertiary doses of an anti-TBEV antibody. For example, In some embodiments, only a single secondary dose is administered to the patient. In other embodiments, two or more (e.g., 2, 3, 4, 5, 6, 7, 8, or more) secondary doses are administered to the patient. Likewise, In some embodiments, only a single tertiary dose is administered to the patient. In other embodiments, two or more (e.g., 2, 3, 4, 5, 6, 7, 8, or more) tertiary doses are administered to the patient.

In some embodiments of the invention, the frequency at which the secondary and/or tertiary doses are administered to a patient can vary over the course of the treatment regimen. The frequency of administration may also be adjusted during the course of treatment by a physician depending on the needs of the individual patient following clinical examination.

Diagnostic Uses of the Antibodies

The disclosed anti-TBEV antibodies may be used to detect and/or measure a tick-borne flavivirus (e.g., TBEV) in a sample, e.g., for diagnostic purposes. Some embodiments contemplate the use of one or more antibodies of the present invention in assays to detect a tick-borne flavivirus (e.g., TBEV)-associated disease or disorder. Exemplary diagnostic assays for TBEV may comprise, e.g., contacting a sample, obtained from a patient, with an anti-TBEV antibody, wherein the anti-TBEV antibody is labeled with a detectable label or reporter molecule or used as a capture ligand to selectively isolate TBEV from patient samples. Alternatively, an unlabeled anti-TBEV antibody can be used in diagnostic applications in combination with a secondary antibody, which is itself detectably labeled. The detectable label or reporter molecule can be a radioisotope, such as H, C, P, S, or I; a fluorescent or chemiluminescent moiety such as fluorescein isothiocyanate, or rhodamine; or an enzyme such as alkaline phosphatase, β-galactosidase, horseradish peroxidase, or luciferase. Specific exemplary assays that can be used to detect or measure a tick-borne flavivirus (e.g., TBEV) in a sample include enzyme-linked immunosorbent assay (ELISA), radioimmunoassay (RIA), and fluorescence-activated cell sorting (FACS).

In another aspect, this disclosure further provides a method for detecting the presence of a tick-borne flavivirus (e.g., TBEV) in a sample comprising the steps of: (i) contacting a sample with the antibody or antigen-binding fragment thereof described above; and (ii) determining binding of the antibody or antigen-binding fragment to one or more tick-borne flavivirus (e.g., TBEV) antigens, wherein binding of the antibody to the one or more tick-borne flavivirus (e.g., TBEV) antigens is indicative of the presence of the tick-borne flavivirus (e.g., TBEV) in the sample.

In some embodiments, the antibody or antigen-binding fragment thereof is conjugated to a label. In some embodiments, the step of detecting comprises contacting a secondary antibody with the antibody or antigen-binding fragment thereof and wherein the secondary antibody comprises a label. In some embodiments, the label includes a fluorescent label, a chemiluminescent label, a radiolabel, and an enzyme.

In some embodiments, the step of detecting comprises detecting fluorescence or chemiluminescence. In some embodiments, the step of detecting comprises a competitive binding assay or ELISA.

In some embodiments, the method further comprises binding the sample to a solid support. In some embodiments, the solid support includes microparticles, microbeads, magnetic beads, and an affinity purification column.

Samples that can be used in tick-borne flavivirus (e.g., TBEV) diagnostic assays include any tissue or fluid sample obtainable from a patient, which contains detectable quantities of either a tick-borne flavivirus (e.g., TBEV) protein or fragments thereof, under normal or pathological conditions. Generally, levels of TBEV protein in a particular sample obtained from a healthy patient (e.g., a patient not afflicted with a disease associated with a tick-borne flavivirus (e.g., TBEV)) will be measured to initially establish a baseline, or standard, level of the tick-borne flavivirus (e.g., TBEV). This baseline level of the tick-borne flavivirus (e.g., TBEV) can then be compared against the levels of the tick-borne flavivirus (e.g., TBEV) measured in samples obtained from individuals suspected of having a tick-borne flavivirus (e.g., TBEV)-associated condition, or symptoms associated with such condition.

The antibodies specific for a tick-borne flavivirus (e.g., TBEV) protein may contain no additional labels or moieties, or they may contain an N-terminal or C-terminal label or moiety. In one embodiment, the label or moiety is biotin. In a binding assay, the location of a label (if any) may determine the orientation of the peptide relative to the surface upon which the peptide is bound. For example, if a surface is coated with avidin, a peptide containing an N-terminal biotin will be oriented such that the C-terminal portion of the peptide will be distal to the surface.

### D. Kits

In another aspect, this disclosure provides a kit comprising a pharmaceutically acceptable dose unit of the antibody or antigen-binding fragment thereof of or the pharmaceutical composition as described above. Also within the scope of this disclosure is a kit for the diagnosis, prognosis or monitoring the treatment of tick-borne flavivirus (e.g., TBEV)-associated infections or diseases in a subject, comprising: the antibody or antigen-binding fragment thereof as described; and a least one detection reagent that binds specifically to the antibody or antigen-binding fragment thereof.

In some embodiments, the kit also includes a container that contains the composition and optionally informational material. The informational material can be descriptive, instructional, marketing or other material that relates to the methods described herein and/or the use of the agents for therapeutic benefit. In an embodiment, the kit also includes an additional therapeutic agent, as described above. For example, the kit includes a first container that contains the composition and a second container for the additional therapeutic agent.

The informational material of the kits is not limited in its form. In some embodiments, the informational material can include information about production of the composition, concentration, date of expiration, batch or production site information, and so forth. In one embodiment, the informational material relates to methods of administering the composition, e.g., in a suitable dose, dosage form, or mode of administration (e.g., a dose, dosage form, or mode of administration described herein), to treat a subject in need thereof. In one embodiment, the instructions provide a dosing regimen, dosing schedule, and/or route of administration of the composition or the additional therapeutic agent. The information can be provided in a variety of formats, including printed text, computer-readable material, video recording, or audio recording, or information that contains a link or address to substantive material.

The kit can include one or more containers for the composition. In some embodiments, the kit contains separate containers, dividers or compartments for the composition and informational material. For example, the composition can be contained in a bottle or vial, and the informational material can be contained in a plastic sleeve or packet. In other embodiments, the separate elements of the kit are contained within a single, undivided container. For example, the composition is contained in a bottle or vial that has attached thereto the informational material in the form of a label. In some embodiments, the kit includes a plurality (e.g., a pack) of individual containers, each containing one or more unit dosage forms (e.g., a dosage form described herein) of the agents.

The kit optionally includes a device suitable for administration of the composition or other suitable delivery device. The device can be provided pre-loaded with one or both of the agents or can be empty, but suitable for loading. Such a kit may optionally contain a syringe to allow for injection of the antibody contained within the kit into an animal, such as a human.

### E. Definitions

To aid in understanding the detailed description of the compositions and methods according to the disclosure, a few express definitions are provided to facilitate an unambiguous disclosure of the various aspects of the disclosure. Unless otherwise defined, all technical and scientific terms used herein have the same meaning as commonly understood by one of ordinary skill in the art to which this disclosure belongs.

The term “antibody” as referred to herein includes whole antibodies and any antigen-binding fragment or single chains thereof. Whole antibodies are glycoproteins comprising at least two heavy (H) chains and two light (L) chains inter-connected by disulfide bonds. Each heavy chain is comprised of a heavy chain variable region (abbreviated herein as VH) and a heavy chain constant region. The heavy chain constant region is comprised of three domains, CH1, CH2 and CH3. Each light chain is comprised of a light chain variable region (abbreviated herein as VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. The heavy chain variable region CDRs and FRs are HFR1, HCDR1, HFR2, HCDR2, HFR3, HCDR3, HFR4. The light chain variable region CDRs and FRs are LFR1, LCDR1, LFR2, LCDR2, LFR3, LCDR3, LFR4. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (CIq) of the classical complement system.

The term “antigen-binding fragment or portion” of an antibody (or simply “antibody fragment or portion”), as used herein, refers to one or more fragments of an antibody that retain the ability to specifically bind to an antigen. It has been shown that the antigen-binding function of an antibody can be performed by fragments of a full-length antibody. Examples of binding fragments encompassed within the term “antigen-binding fragment or portion” of an antibody include (i) a Fab fragment, a monovalent fragment consisting of the VL, VH, CL and CHI domains; (ii) a F(ab′)2 fragment, a bivalent fragment comprising two Fab fragments linked by a disulfide bridge at the hinge region; (iii) a Fab′ fragment, which is essentially a Fab with part of the hinge region (see, FUNDAMENTAL IMMUNOLOGY (Paul ed., 3rd ed. 1993)); (iv) a Fd fragment consisting of the VH and CHI domains; (v) a Fv fragment consisting of the VL and VH domains of a single arm of an antibody, (vi) a dAb fragment (Ward et al., (1989) Nature 341:544-546), which consists of a VH domain; (vii) an isolated CDR; and (viii) a nanobody, a heavy chain variable region containing a single variable domain and two constant domains. Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv or scFv); see, e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding fragment or portion” of an antibody. These antibody fragments are obtained using conventional techniques known to those with skill in the art, and the fragments are screened for utility in the same manner as are intact antibodies.

An “isolated antibody,” as used herein, is intended to refer to an antibody that is substantially free of other antibodies having different antigenic specificities. An isolated antibody can be substantially free of other cellular material and/or chemicals.

The terms “monoclonal antibody” or “monoclonal antibody composition” as used herein refer to a preparation of antibody molecules of single molecular composition. A monoclonal antibody composition displays a single binding specificity and affinity for a particular epitope.

The term “human antibody” is intended to include antibodies having variable regions in which both the framework and CDR regions are derived from human germline immunoglobulin sequences. Furthermore, if the antibody contains a constant region, the constant region also is derived from human germline immunoglobulin sequences. The human antibodies of the invention can include amino acid residues not encoded by human germline immunoglobulin sequences (e.g., mutations introduced by random or site-specific mutagenesis in vitro or by somatic mutation in vivo). However, the term “human antibody,” as used herein, is not intended to include antibodies in which CDR sequences derived from the germline of another mammalian species, such as a mouse, have been grafted onto human framework sequences.

The term “human monoclonal antibody” refers to antibodies displaying a single binding specificity, which have variable regions in which both the framework and CDR regions are derived from human germline immunoglobulin sequences. In one embodiment, the human monoclonal antibodies can be produced by a hybridoma that includes a B cell obtained from a transgenic nonhuman animal, e.g., a transgenic mouse, having a genome comprising a human heavy chain transgene and a light chain transgene fused to an immortalized cell.

The term “recombinant human antibody,” as used herein, includes all human antibodies that are prepared, expressed, created or isolated by recombinant means, such as (a) antibodies isolated from an animal (e.g., a mouse) that is transgenic or transchromosomal for human immunoglobulin genes or a hybridoma prepared therefrom (described further below), (b) antibodies isolated from a host cell transformed to express the human antibody, e.g., from a transfectoma, (c) antibodies isolated from a recombinant, combinatorial human antibody library, and (d) antibodies prepared, expressed, created or isolated by any other means that involve splicing of human immunoglobulin gene sequences to other DNA sequences. Such recombinant human antibodies have variable regions in which the framework and CDR regions are derived from human germline immunoglobulin sequences. In some embodiments, however, such recombinant human antibodies can be subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

The term “isotype” refers to the antibody class (e.g., IgM or IgG1) that is encoded by the heavy chain constant region genes. The phrases “an antibody recognizing an antigen” and “an antibody specific for an antigen” are used interchangeably herein with the term “an antibody which binds specifically to an antigen.”

The term “human antibody derivatives” refers to any modified form of the human antibody, e.g., a conjugate of the antibody and another agent or antibody. The term “humanized antibody” is intended to refer to antibodies in which CDR sequences derived from the germline of another mammalian species, such as a mouse, have been grafted onto human framework sequences. Additional framework region modifications can be made within the human framework sequences.

The term “chimeric antibody” is intended to refer to antibodies in which the variable region sequences are derived from one species, and the constant region sequences are derived from another species, such as an antibody in which the variable region sequences are derived from a mouse antibody, and the constant region sequences are derived from a human antibody. The term can also refer to an antibody in which its variable region sequence or CDR(s) is derived from one source (e.g., an IgA1 antibody) and the constant region sequence or Fc is derived from a different source (e.g., a different antibody, such as an IgG, IgA2, IgD, IgE or IgM antibody).

The invention encompasses isolated or substantially purified nucleic acids, peptides, polypeptides or proteins. In the context of the present invention, an “isolated” nucleic acid, DNA or RNA molecule or an “isolated” polypeptide is a nucleic acid, DNA molecule, RNA molecule, or polypeptide that exists apart from its native environment and is therefore not a product of nature. An isolated nucleic acid, DNA molecule, RNA molecule or polypeptide may exist in a purified form or may exist in a non-native environment such as, for example, a transgenic host cell. A “purified” nucleic acid molecule, peptide, polypeptide or protein, or a fragment thereof, is substantially free of other cellular material, or culture medium when produced by recombinant techniques, or substantially free of chemical precursors or other chemicals when chemically synthesized. In one embodiment, an “isolated” nucleic acid is free of sequences that naturally flank the nucleic acid (i.e., sequences located at the 5′ and 3′ ends of the nucleic acid) in the genomic DNA of the organism from which the nucleic acid is derived. For example, in various embodiments, the isolated nucleic acid molecule can contain less than about 5 kb, 4 kb, 3 kb, 2 kb, 1 kb, 0.5 kb, or 0.1 kb of nucleotide sequences that naturally flank the nucleic acid molecule in genomic DNA of the cell from which the nucleic acid is derived. A protein, peptide or polypeptide that is substantially free of cellular material includes preparations of protein, peptide or polypeptide having less than about 30%, 20%, 10%, or 5% (by dry weight) of contaminating protein. When the protein of the invention, or biologically active portion thereof, is recombinantly produced, preferably culture medium represents less than about 30%, 20%, 10%, or 5% (by dry weight) of chemical precursors or non-protein-of-interest chemicals.

The terms “polypeptide,” “peptide,” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified; for example, disulfide bond formation, glycosylation, lipidation, acetylation, phosphorylation, pegylation, or any other manipulation, such as conjugation with a labeling component. As used herein, the term “amino acid” includes natural and/or unnatural or synthetic amino acids, including glycine and both the D or L optical isomers, and amino acid analogs and peptidomimetics.

A peptide or polypeptide “fragment” as used herein refers to a less than full-length peptide, polypeptide or protein. For example, a peptide or polypeptide fragment can have is at least about 3, at least about 4, at least about 5, at least about 10, at least about 20, at least about 30, at least about 40 amino acids in length, or single unit lengths thereof. For example, fragment may be 6, 7, 8, 9, 10, 11, 12, 13, 14, 15, 16, 17, or more amino acids in length. There is no upper limit to the size of a peptide fragment. However, in some embodiments, peptide fragments can be less than about 500 amino acids, less than about 400 amino acids, less than about 300 amino acids or less than about 250 amino acids in length. Preferably the peptide fragment can elicit an immune response when used to inoculate an animal. A peptide fragment may be used to elicit an immune response by inoculating an animal with a peptide fragment in combination with an adjuvant, a peptide fragment that is coupled to an adjuvant, or a peptide fragment that is coupled to arsanilic acid, sulfanilic acid, an acetyl group, or a picryl group. A peptide fragment can include a non-amide bond and can be a peptidomimetic.

As used herein, the term “conjugate” or “conjugation” or “linked” as used herein refers to the attachment of two or more entities to form one entity. A conjugate encompasses both peptide-small molecule conjugates as well as peptide-protein/peptide conjugates.

The term “recombinant,” as used herein, refers to antibodies or antigen-binding fragments thereof of the invention created, expressed, isolated or obtained by technologies or methods known in the art as recombinant DNA technology, which include, e.g., DNA splicing and transgenic expression. The term refers to antibodies expressed in a non-human mammal (including transgenic non-human mammals, e.g., transgenic mice), or a cell (e.g., CHO cells) expression system or isolated from a recombinant combinatorial human antibody library.

A “nucleic acid” or “polynucleotide” refers to a DNA molecule (for example, but not limited to, a cDNA or genomic DNA) or an RNA molecule (for example, but not limited to, an mRNA), and includes DNA or RNA analogs. A DNA or RNA analog can be synthesized from nucleotide analogs. The DNA or RNA molecules may include portions that are not naturally occurring, such as modified bases, modified backbone, deoxyribonucleotides in an RNA, etc. The nucleic acid molecule can be single-stranded or double-stranded.

The term “substantial identity” or “substantially identical,” when referring to a nucleic acid or fragment thereof, indicates that, when optimally aligned with appropriate nucleotide insertions or deletions with another nucleic acid (or its complementary strand), there is nucleotide sequence identity in at least about 90%, and more preferably at least about 95%, 96%, 97%, 98% or 99% of the nucleotide bases, as measured by any well-known algorithm of sequence identity, such as FASTA, BLAST or GAP, as discussed below. A nucleic acid molecule having substantial identity to a reference nucleic acid molecule may, in certain instances, encode a polypeptide having the same or substantially similar amino acid sequence as the polypeptide encoded by the reference nucleic acid molecule.

As applied to polypeptides, the term “substantial similarity” or “substantially similar” means that two peptide sequences, when optimally aligned, such as by the programs GAP or BESTFIT using default gap weights, share at least 90% sequence identity, even more preferably at least 95%, 98% or 99% sequence identity. Preferably, residue positions, which are not identical, differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent or degree of similarity may be adjusted upwards to correct for the conservative nature of the substitution. Means for making this adjustment are well known to those of skill in the art. See, e.g., Pearson (1994) Methods Mol. Biol. 24: 307-331, which is herein incorporated by reference. Examples of groups of amino acids that have side chains with similar chemical properties include 1) aliphatic side chains: glycine, alanine, valine, leucine, and isoleucine; 2) aliphatic-hydroxyl side chains: serine and threonine; 3) amide-containing side chains: asparagine and glutamine; 4) aromatic side chains: phenylalanine, tyrosine, and tryptophan; 5) basic side chains: lysine, arginine, and histidine; 6) acidic side chains: aspartate and glutamate, and 7) sulfur-containing side chains: cysteine and methionine. Preferred conservative amino acids substitution groups are: valine-leucine-isoleucine, phenylalanine-tyrosine, lysine-arginine, alanine-valine, glutamate-aspartate, and asparagine-glutamine. Alternatively, a conservative replacement is any change having a positive value in the PAM250 log-likelihood matrix disclosed in Gonnet et al. (1992) Science 256: 1443 45, herein incorporated by reference. A “moderately conservative” replacement is any change having a nonnegative value in the PAM250 log-likelihood matrix.

Sequence similarity for polypeptides is typically measured using sequence analysis software. Protein analysis software matches similar sequences using measures of similarity assigned to various substitutions, deletions, and other modifications, including conservative amino acid substitutions. For instance, GCG software contains programs such as GAP and BESTFIT, which can be used with default parameters to determine sequence homology or sequence identity between closely related polypeptides, such as homologous polypeptides from different species of organisms or between a wild type protein and a mutein thereof. See, e.g., GCG Version 6.1. Polypeptide sequences also can be compared using FASTA with default or recommended parameters; a program in GCG Version 6.1. FASTA (e.g., FASTA2 and FASTA3) provides alignments and percent sequence identity of the regions of the best overlap between the query and search sequences (Pearson (2000) supra). Another preferred algorithm when comparing a sequence of the invention to a database containing a large number of sequences from different organisms is the computer program BLAST, especially BLASTP or TBLASTN, using default parameters. See, e.g., Altschul et al. (1990) J. Mol. Biol. 215: 403-410 and (1997) Nucleic Acids Res. 25:3389-3402, each of which is herein incorporated by reference.

As used herein, the term “affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity, which reflects a 1:1 interaction between members of a binding pair (e.g., antibody and antigen). The affinity of a molecule X for its partner Y can generally be represented by the dissociation constant (KD). Affinity can be measured by common methods known in the art, including those described herein.

The term “specifically binds,” or “binds specifically to,” or the like, refers to an antibody that binds to a single epitope, e.g., under physiologic conditions, but which does not bind to more than one epitope. Accordingly, an antibody that specifically binds to a polypeptide will bind to an epitope that present on the polypeptide, but which is not present on other polypeptides. Specific binding can be characterized by an equilibrium dissociation constant of at least about 1×10⁻⁸ M or less (e.g., a smaller KD denotes a tighter binding). Methods for determining whether two molecules specifically bind are well known in the art and include, for example, equilibrium dialysis, surface plasmon resonance, and the like.

For example, the antibody binds to an epitope with “high affinity,” namely with a KD of 1×10⁻⁷ M or less, more preferably 5×10⁻⁸ M or less, more preferably 3×10⁻⁸ M or less, more preferably 1×10⁻⁸ M or less, more preferably 5×10-9 M or less or even more preferably 1×10⁻⁹ M or less, as determined by surface plasmon resonance, e.g., BIACORE. The term “does not substantially bind” to a protein or cells, as used herein, means does not bind or does not bind with a high affinity to the protein or cells, i.e., binds to the protein or cells with a KD of 1×10⁻⁶ M or more, more preferably 1×10⁻⁵ M or more, more preferably 1×10⁻⁴ M or more, more preferably 1×10⁻³ M or more, even more preferably 1×10⁻² M or more.

The term “Kassoc” or “Ka,” as used herein, is intended to refer to the association rate of a particular antibody-antigen interaction, whereas the term “Kdis” or “Kd,” as used herein, is intended to refer to the dissociation rate of a particular antibody-antigen interaction. The term “KD,” as used herein, is intended to refer to the dissociation constant, which is obtained from the ratio of Kd to Ka (i.e., Kd/Ka) and is expressed as a molar concentration (M). KD values for antibodies can be determined using methods well established in the art. A preferred method for determining the KD of an antibody is by using surface plasmon resonance, preferably using a biosensor system such as a BIACORE system.

Antibodies that “compete with another antibody for binding to a target” refer to antibodies that inhibit (partially or completely) the binding of the other antibody to the target. Whether two antibodies compete with each other for binding to a target, i.e., whether and to what extent one antibody inhibits the binding of the other antibody to a target, may be determined using known competition experiments. In some embodiments, an antibody competes with, and inhibits binding of another antibody to a target by at least 10%, 20%, 30%, 40%, 50%, 60%, 70%, 80%, 90% or 100%. The level of inhibition or competition may be different depending on which antibody is the “blocking antibody” (i.e., the cold antibody that is incubated first with the target). Competition assays can be conducted as described, for example, in Ed Harlow and David Lane, Cold Spring Harb Protoc; 2006 or in Chapter 11 of “Using Antibodies” by Ed Harlow and David Lane, Cold Spring Harbor Laboratory Press, Cold Spring Harbor, NY, USA 1999. Competing antibodies bind to the same epitope, an overlapping epitope or to adjacent epitopes (e.g., as evidenced by steric hindrance). Other competitive binding assays include: solid-phase direct or indirect radioimmunoassay (RIA), solid-phase direct or indirect enzyme immunoassay (EIA), sandwich competition assay (see Stahli et al., Methods in Enzymology 9:242 (1983)); solid-phase direct biotin-avidin EIA (see Kirkland et al., J. Immunol. 137:3614 (1986)); solid-phase direct labeled assay, solid-phase direct labeled sandwich assay (see Harlow and Lane, Antibodies: A Laboratory Manual, Cold Spring Harbor Press (1988)); solid-phase direct label RIA using 1-125 label (see Morel et al., Mol. Immunol. 25(1):7 (1988)); solid-phase direct biotin-avidin EIA (Cheung et al., Virology 176:546 (1990)); and direct labeled RIA. (Moldenhauer et al., Scand. J. Immunol. 32:77 (1990)).

The term “epitope” as used herein refers to an antigenic determinant that interacts with a specific antigen-binding site in the variable region of an antibody molecule known as a paratope. A single antigen may have more than one epitope. Thus, different antibodies may bind to different areas on an antigen and may have different biological effects. The term “epitope” also refers to a site on an antigen to which B and/or T cells respond. It also refers to a region of an antigen that is bound by an antibody. Epitopes may be defined as structural or functional. Functional epitopes are generally a subset of the structural epitopes and have those residues that directly contribute to the affinity of the interaction. Epitopes may also be conformational, that is, composed of non-linear amino acids. In some embodiments, epitopes may include determinants that are chemically active surface groupings of molecules such as amino acids, sugar side chains, phosphoryl groups, or sulfonyl groups, and, In some embodiments, may have specific three-dimensional structural characteristics and/or specific charge characteristics. An epitope typically includes at least 3, 4, 5, 6, 7, 8, 9, 10, 11, 12, 13, 14 or 15 amino acids in a unique spatial conformation. Methods for determining what epitopes are bound by a given antibody (i.e., epitope mapping) are well known in the art. Methods of determining spatial conformation of epitopes include techniques in the art and those described herein, for example, x-ray crystallography and 2-dimensional nuclear magnetic resonance (see, e.g., Epitope Mapping Protocols in Methods in Molecular Biology, Vol. 66, G. E. Morris, Ed. (1996)).

The term “epitope mapping” refers to the process of identification of the molecular determinants for antibody-antigen recognition.

The term “binds to an epitope” or “recognizes an epitope” with reference to an antibody or antibody fragment refers to continuous or discontinuous segments of amino acids within an antigen. Those of skill in the art understand that the terms do not necessarily mean that the antibody or antibody fragment is in direct contact with every amino acid within an epitope sequence.

The term “binds to the same epitope” with reference to two or more antibodies means that the antibodies bind to the same, overlapping or encompassing continuous or discontinuous segments of amino acids. Those of skill in the art understand that the phrase “binds to the same epitope” does not necessarily mean that the antibodies bind to or contact exactly the same amino acids. The precise amino acids that the antibodies contact can differ. For example, a first antibody can bind to a segment of amino acids that is completely encompassed by the segment of amino acids bound by a second antibody. In another example, a first antibody binds one or more segments of amino acids that significantly overlap the one or more segments bound by the second antibody. For the purposes herein, such antibodies are considered to “bind to the same epitope.”

As used herein, the term “immune response” refers to a biological response within a vertebrate against foreign agents, which response protects the organism against these agents and diseases caused by them. An immune response is mediated by the action of a cell of the immune system (for example, a T lymphocyte, B lymphocyte, natural killer (NK) cell, macrophage, eosinophil, mast cell, dendritic cell or neutrophil) and soluble macromolecules produced by any of these cells or the liver (including antibodies, cytokines, and complement) that results in selective targeting, binding to, damage to, destruction of, and/or elimination from the vertebrate's body of invading pathogens, cells or tissues infected with pathogens, cancerous or other abnormal cells, or, in cases of autoimmunity or pathological inflammation, normal human cells or tissues. An immune reaction includes, e.g., activation or inhibition of a T cell, e.g., an effector T cell or a Th cell, such as a CD4+ or CD8+ T cell, or the inhibition of a Treg cell.

The term “detectable label” as used herein refers to a molecule capable of detection, including, but not limited to, radioactive isotopes, fluorescers, chemiluminescers, chromophores, enzymes, enzyme substrates, enzyme cofactors, enzyme inhibitors, chromophores, dyes, metal ions, metal sols, ligands (e.g., biotin, avidin, streptavidin or haptens), intercalating dyes and the like. The term “fluorescer” refers to a substance or a portion thereof that is capable of exhibiting fluorescence in the detectable range.

In many embodiments, the terms “subject” and “patient” are used interchangeably irrespective of whether the subject has or is currently undergoing any form of treatment. As used herein, the terms “subject” and “subjects” may refer to any vertebrate, including, but not limited to, a mammal (e.g., cow, pig, camel, llama, horse, goat, rabbit, sheep, hamsters, guinea pig, cat, dog, rat, and mouse, a non-human primate (for example, a monkey, such as a cynomolgus monkey, chimpanzee, etc.) and a human). The subject may be a human or a non-human. In more exemplary aspects, the mammal is a human. As used herein, the expression “a subject in need thereof” or “a patient in need thereof” means a human or non-human mammal that exhibits one or more symptoms or indications of disorders (e.g., neuronal disorders, autoimmune diseases, and cardiovascular diseases), and/or who has been diagnosed with inflammatory disorders. In some embodiments, the subject is a mammal. In some embodiments, the subject is human.

As used herein, the term “disease” is intended to be generally synonymous and is used interchangeably with, the terms “disorder” and “condition” (as in medical condition), in that all reflect an abnormal condition (e.g., inflammatory disorder) of the human or animal body or of one of its parts that impairs normal functioning, is typically manifested by distinguishing signs and symptoms, and causes the human or animal to have a reduced duration or quality of life.

As used herein, the term “treating” or “treatment” of any disease or disorder refers in one embodiment, to ameliorating the disease or disorder (i.e., arresting or reducing the development of the disease or at least one of the clinical symptoms thereof). In another embodiment, “treating” or “treatment” refers to ameliorating at least one physical parameter, which may not be discernible by the patient. In yet another embodiment, “treating” or “treatment” refers to modulating the disease or disorder, either physically, (e.g., stabilization of a discernible symptom), physiologically, (e.g., stabilization of a physical parameter), or both. In yet another embodiment, “treating” or “treatment” refers to preventing or delaying the onset or development or progression of the disease or disorder.

The terms “prevent,” “preventing,” “prevention,” “prophylactic treatment” and the like refer to reducing the probability of developing a disorder or condition in a subject, who does not have, but is at risk of or susceptible to developing a disorder or condition.

The terms “decrease,” “reduced,” “reduction,” “decrease,” or “inhibit” are all used herein generally to mean a decrease by a statistically significant amount. However, for avoidance of doubt, “reduced,” “reduction” or “decrease” or “inhibit” means a decrease by at least 10% as compared to a reference level, for example, a decrease by at least about 20%, or at least about 30%, or at least about 40%, or at least about 50%, or at least about 60%, or at least about 70%, or at least about 80%, or at least about 90% or up to and including a 100% decrease (e.g., absent level as compared to a reference sample), or any decrease between 10-100% as compared to a reference level.

As used herein, the term “agent” denotes a chemical compound, a mixture of chemical compounds, a biological macromolecule (such as a nucleic acid, an antibody, a protein or portion thereof, e.g., a peptide), or an extract made from biological materials such as bacteria, plants, fungi, or animal (particularly mammalian) cells or tissues. The activity of such agents may render it suitable as a “therapeutic agent,” which is a biologically, physiologically, or pharmacologically active substance (or substances) that acts locally or systemically in a subject.

As used herein, the terms “therapeutic agent,” “therapeutic capable agent,” or “treatment agent” are used interchangeably and refer to a molecule or compound that confers some beneficial effect upon administration to a subject. The beneficial effect includes enablement of diagnostic determinations; amelioration of a disease, symptom, disorder, or pathological condition; reducing or preventing the onset of a disease, symptom, disorder, or condition; and generally counteracting a disease, symptom, disorder or pathological condition.

The term “therapeutic effect” is art-recognized and refers to a local or systemic effect in animals, particularly mammals, and more particularly humans caused by a pharmacologically active substance.

The term “effective amount,” “effective dose,” or “effective dosage” is defined as an amount sufficient to achieve or at least partially achieve a desired effect. A “therapeutically effective amount” or “therapeutically effective dosage” of a drug or therapeutic agent is any amount of the drug that, when used alone or in combination with another therapeutic agent, promotes disease regression evidenced by a decrease in severity of disease symptoms, an increase in frequency and duration of disease symptom-free periods, or a prevention of impairment or disability due to the disease affliction. A “prophylactically effective amount” or a “prophylactically effective dosage” of a drug is an amount of the drug that, when administered alone or in combination with another therapeutic agent to a subject at risk of developing a disease or of suffering a recurrence of disease, inhibits the development or recurrence of the disease. The ability of a therapeutic or prophylactic agent to promote disease regression or inhibit the development or recurrence of the disease can be evaluated using a variety of methods known to the skilled practitioner, such as in human subjects during clinical trials, in animal model systems predictive of efficacy in humans, or by assaying the activity of the agent in in vitro assays.

Doses are often expressed in relation to bodyweight. Thus, a dose which is expressed as [g, mg, or other unit]/kg (or g, mg etc.) usually refers to [g, mg, or other unit] “per kg (or g, mg etc.) bodyweight,” even if the term “bodyweight” is not explicitly mentioned.

As used herein, the term “composition” or “pharmaceutical composition” refers to a mixture of at least one component useful within the invention with other components, such as carriers, stabilizers, diluents, dispersing agents, suspending agents, thickening agents, and/or excipients. The pharmaceutical composition facilitates administration of one or more components of the invention to an organism.

As used herein, the term “pharmaceutically acceptable” refers to a material, such as a carrier or diluent, which does not abrogate the biological activity or properties of the composition, and is relatively non-toxic, i.e., the material may be administered to an individual without causing undesirable biological effects or interacting in a deleterious manner with any of the components of the composition in which it is contained.

As used herein, the term “pharmaceutically acceptable carrier” includes a pharmaceutically acceptable salt, pharmaceutically acceptable material, composition or carrier, such as a liquid or solid filler, diluent, excipient, solvent or encapsulating material, involved in carrying or transporting a compound(s) of the present invention within or to the subject such that it may perform its intended function. Typically, such compounds are carried or transported from one organ, or portion of the body, to another organ, or portion of the body. Each salt or carrier must be “acceptable” in the sense of being compatible with the other ingredients of the formulation and not injurious to the subject. Some examples of materials that may serve as pharmaceutically acceptable carriers include: sugars, such as lactose, glucose, and sucrose; starches, such as corn starch and potato starch; cellulose, and its derivatives, such as sodium carboxymethyl cellulose, ethyl cellulose, and cellulose acetate; powdered tragacanth; malt; gelatin; talc; excipients, such as cocoa butter and suppository waxes; oils, such as peanut oil, cottonseed oil, safflower oil, sesame oil, olive oil, corn oil, and soybean oil; glycols, such as propylene glycol; polyols, such as glycerin, sorbitol, mannitol, and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; diluent; granulating agent; lubricant; binder; disintegrating agent; wetting agent; emulsifier; coloring agent; release agent; coating agent; sweetening agent; flavoring agent; perfuming agent; preservative; antioxidant; plasticizer; gelling agent; thickener; hardener; setting agent; suspending agent; surfactant; humectant; carrier; stabilizer; and other non-toxic compatible substances employed in pharmaceutical formulations, or any combination thereof. As used herein, “pharmaceutically acceptable carrier” also includes any and all coatings, antibacterial and antifungal agents, and absorption delaying agents, and the like that are compatible with the activity of one or more components of the invention, and are physiologically acceptable to the subject. Supplementary active compounds may also be incorporated into the compositions.

“Combination” therapy, as used herein, unless otherwise clear from the context, is meant to encompass administration of two or more therapeutic agents in a coordinated fashion and includes, but is not limited to, concurrent dosing. Specifically, combination therapy encompasses both co-administration (e.g., administration of a co-formulation or simultaneous administration of separate therapeutic compositions) and serial or sequential administration, provided that administration of one therapeutic agent is conditioned in some way on the administration of another therapeutic agent. For example, one therapeutic agent may be administered only after a different therapeutic agent has been administered and allowed to act for a prescribed period of time. See, e.g., Kohrt et al. (2011) Blood 117:2423.

As used herein, the term “co-administration” or “co-administered” refers to the administration of at least two agent(s) or therapies to a subject. In some embodiments, the co-administration of two or more agents/therapies is concurrent. In other embodiments, a first agent/therapy is administered prior to a second agent/therapy. Those of skill in the art understand that the formulations and/or routes of administration of the various agents/therapies used may vary.

As used herein, the term “contacting,” when used in reference to any set of components, includes any process whereby the components to be contacted are mixed into the same mixture (for example, are added into the same compartment or solution), and does not necessarily require actual physical contact between the recited components. The recited components can be contacted in any order or any combination (or sub-combination) and can include situations where one or some of the recited components are subsequently removed from the mixture, optionally prior to addition of other recited components. For example, “contacting A with B and C” includes any and all of the following situations: (i) A is mixed with C, then B is added to the mixture; (ii) A and B are mixed into a mixture; B is removed from the mixture, and then C is added to the mixture; and (iii) A is added to a mixture of B and C.

“Sample,” “test sample,” and “patient sample” may be used interchangeably herein. The sample can be a sample of serum, urine plasma, amniotic fluid, cerebrospinal fluid, cells, or tissue. Such a sample can be used directly as obtained from a patient or can be pre-treated, such as by filtration, distillation, extraction, concentration, centrifugation, inactivation of interfering components, addition of reagents, and the like, to modify the character of the sample in some manner as discussed herein or otherwise as is known in the art. The terms “sample” and “biological sample” as used herein generally refer to a biological material being tested for and/or suspected of containing an analyte of interest such as antibodies. The sample may be any tissue sample from the subject. The sample may comprise protein from the subject.

As used herein, the term “in vitro” refers to events that occur in an artificial environment, e.g., in a test tube or reaction vessel, in cell culture, etc., rather than within a multi-cellular organism.

As used herein, the term “in vivo” refers to events that occur within a multi-cellular organism, such as a non-human animal.

As used herein, the singular forms “a,” “an,” and “the” include plural references unless the context clearly dictates otherwise.

As used herein, the terms “including,” “comprising,” “containing,” or “having” and variations thereof are meant to encompass the items listed thereafter and equivalents thereof as well as additional subject matter unless otherwise noted.

As used herein, the phrases “in one embodiment,” “in various embodiments,” “in some embodiments,” and the like are used repeatedly. Such phrases do not necessarily refer to the same embodiment, but they may unless the context dictates otherwise.

As used herein, the terms “and/or” or “/” means any one of the items, any combination of the items, or all of the items with which this term is associated.

As used herein, the word “substantially” does not exclude “completely,” e.g., a composition which is “substantially free” from Y may be completely free from Y. Where necessary, the word “substantially” may be omitted from the definition of the invention.

As used herein, the term “each,” when used in reference to a collection of items, is intended to identify an individual item in the collection but does not necessarily refer to every item in the collection. Exceptions can occur if explicit disclosure or context clearly dictates otherwise.

As used herein, the term “approximately” or “about,” as applied to one or more values of interest, refers to a value that is similar to a stated reference value. In some embodiments, the term “approximately” or “about” refers to a range of values that fall within 25%, 20%, 19%, 18%, 17%, 16%, 15%, 4%1, 3%1, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). Unless indicated otherwise herein, the term “about” is intended to include values, e.g., weight percents, proximate to the recited range that are equivalent in terms of the functionality of the individual ingredient, the composition, or the embodiment.

As disclosed herein, a number of ranges of values are provided. It is understood that each intervening value, to the tenth of the unit of the lower limit, unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither, or both limits are included in the smaller ranges is also encompassed within the invention, subject to any specifically excluded limit in the stated range. Where the stated range includes one or both of the limits, ranges excluding either or both of those included limits are also included in the invention.

The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

All methods described herein are performed in any suitable order unless otherwise indicated herein or otherwise clearly contradicted by context. In regard to any of the methods provided, the steps of the method may occur simultaneously or sequentially. When the steps of the method occur sequentially, the steps may occur in any order, unless noted otherwise. In cases in which a method comprises a combination of steps, each and every combination or sub-combination of the steps is encompassed within the scope of the disclosure, unless otherwise noted herein.

Each publication, patent application, patent, and other reference cited herein is incorporated by reference in its entirety to the extent that it is not inconsistent with the present disclosure. Publications disclosed herein are provided solely for their disclosure prior to the filing date of the present invention. Nothing herein is to be construed as an admission that the present invention is not entitled to antedate such publication by virtue of prior invention. Further, the dates of publication provided may be different from the actual publication dates, which may need to be independently confirmed.

It is understood that the examples and embodiments described herein are for illustrative purposes only and that various modifications or changes in light thereof will be indicated to persons skilled in the art and are to be included within the spirit and purview of this application and scope of the appended claims.

### F. Examples

### Example 1

This example describes the materials and methods used in the subsequent EXAMPLES below.

Human Subjects and Clinical Information

Samples of peripheral blood were obtained upon consent from individuals previously hospitalized with confirmed TBEV infection or from individuals previously vaccinated against TBEV in České Budějovice, Czech Republic, under protocols approved by the ethical committees of the Hospital in České Budějovice (approval No. 103/19), the Biology Center of the Czech Academy of Sciences (approval No. 1/2018) and the Rockefeller University (IRB DRO-0984). Clinical data were obtained at the treating hospital, and severity of disease was evaluated according to the following scale: mild, flu-like symptoms with meningeal irritation defined as meningitis, characterized by fever, fatigue, nausea, headache, back pain, arthralgia/myalgia, and neck or back stiffness; moderate, previous symptoms together with tremor, vertigo, somnolence and photophobia defined as meningoencephalitis; severe, prolonged neurological consequences including ataxia, titubation, altered mental status, memory loss, quantitative disturbance of consciousness, and palsy revealed as encephalitis, encephalomyelitis, or encephalomyeloradiculitis (Bogovic, P., and Strle, F., (2015) World J Clin Cases 3, 430-441; Ruzek, D., et al., (2019) Antiviral Res 164, 23-51).

Blood Samples Processing and Storage

Peripheral Blood Mononuclear Cells (PBMCs) were obtained by gradient centrifugation using Ficoll and stored in liquid nitrogen in freezing media (90% FCS, 10% DMSO). Prior to experiments, aliquots of sera (from infected, vaccinated, and random blood bank donors) were heat-inactivated at 56° C. for 1 hour and then stored at 4° C.

Protein Expression and Purification

EDIII antigens were expressed in E. coli and purified from inclusion bodies as previously reported (Robbiani, D. F., et al., (2017) Cell 169, 597-609 e511); Sapparapu, G., et al., (2016) Nature 540, 443-447). Expression vectors containing codon-optimized sequences encoding residues 299-397 for TBEV strain Neudoerfl (TBEV^{WE}; NC_001672.1) or 301-397 for strains Sofjin (TBEV^{FE}; UniProtKB P07720) and Vasilchenko (TBEV^{Si}; AF069066) were used to produce untagged EDIII proteins or EDIII proteins containing a C-terminal 6×His-Avitag. Constructs encoding untagged EDIIIs of other tick-borne flaviviruses were constructed similarly (POWV strain LB, GenBank L04636.1; POWV isolate DTV; KFDV strain W-377, JF416960.1; LGTV strain TP21-636, NC_003690.1; LIV isolate LI3/1, KP144331.1; OHFV strain Bogoluvovska, NC_005062). Expression plasmids were transformed into BL21(DE3) E. coli and induced with 1 mM isopropyl β-D-1-thiogalactopyranoside (IPTG) at 37° C. for 4 hours. The cells were lysed and the insoluble fraction containing inclusion bodies was solubilized and refolded in 400 mM L-arginine, 100 mM Tris-base pH 8.0, 2 mM EDTA, 0.2 mM phenyl-methylsulfonyl fluoride, 5 mM reduced and 0.5 mM oxidized glutathione, and 10% glycerol at 4° C. Refolded protein was purified by size exclusion chromatography (Superdex 75; Cytiva) in 20 mM Tris pH 8.0, 150 mM NaCl, 0.02% NaN₃. EDIIIs were concentrated to 10-20 mg/mL.

T025 F(ab)s for structural studies were produced and purified as described in previous studies (Keeffe, J. R., et al., (2018) Cell Rep 25, 1385-1394.e1387; Robbiani, D. F., et al., (2017) Cell 169, 597-609 e511; Robbiani, D. F., et al., (2020) Nature 584, 437-442; Wang, Q., et al., (2020) Cell Host Microbe 28, 335-349.e336). Briefly, F(ab)s containing a 6×His purification tag (SEQ ID NO: 6668) at the C-terminus of the heavy chain were expressed by transiently transfecting Expi293 cells (Life Technologies) with appropriate heavy and light chain plasmids. His-tagged F(ab)s were purified from expression supernatants using Ni-NTA affinity chromatography (Cytiva) followed by size exclusion chromatography (Superdex 200; Cytiva) in 20 mM Tris pH 8.0, 150 mM NaCl, 0.02% NaN₃. Fabs were concentrated to approximately 15 mg/mL.

Sequence Analysis

Antibody sequences were analyzed as described previously (Robbiani, D. F., et al., (2020) Nature 584, 437-442); in particular, sequences were trimmed and annotated using Igblastn v.1.14.0 (Ye, J., et al., (2013) Nucleic Acids Research 41, W34-W40) and Change-O toolkit v.0.4.5 (Gupta, N. T., et al., (2015) Bioinformatics 31, 3356-3358). Sequences from the same cell were paired and assigned clonotypes based on V and J genes using in-house R and Perl scripts, available on GitHub (https://github.com/stratust/igpipeline). Nucleotide somatic hypermutation and CDR3 length were also analyzed using in-house R and Perl scripts, as described previously (Robbiani, D. F., et al., (2020) Nature 584, 437-442); hypermutation analysis was based on the closest germlines in Igblastn. Hydrophobicity GRAVY scores were calculated using Guy H. R. Hydrophobicity scale (Guy, H. R. (1985) Biophysical Journal 47, 61-70; Kyte, J., and Doolittle, R. F., (1982) J Mol Biol 157, 105-132) and R package Peptides (https.//journal.r-project.org/archive/2015/RJ-2015-001/RJ-2015-001.pdf), based on 776 IGH CDR3 sequences from this study and 22,654,256 IGH CDR3 sequences from public databases of memory B cell receptor sequences (DeWitt, W. S., et al., (2016) PLOS ONE 11, e0160853). Distribution was determined using the Shapiro-Wilk test with all CDR3 sequence GRAVY scores from this study and 5,000 randomly selected GRAVY scores from the public database. The Wilcoxon nonparametric test was used to test for the significant difference in hydrophobicity.

Frequency distributions of V genes in anti-TBEV antibodies from 6 infected donors were compared to Sequence Read Archive accession SRP010970 (Rubelt, F., et al., (2012) PLoS One 7, e49774). V gene assignments were based on the above-described analysis, and frequencies were calculated for 6 infected donors using sequences with unique CDR3s. Statistical significance was determined using two-tailed t-tests with unequal variances. Sequence logos were generated from left-aligned CDR3 sequences from each antibody set using WebLogo (Crooks, G. E., et al., (2004) Genome Res 14, 1188-1190).

Protein Biotinylation

Avi-tagged TBEV^{FE} EDIII was biotinylated using the Biotin-Protein Ligase BIRA kit according to the manufacturer's instructions (Avidity) and conjugated to streptavidin-PE (BD Biosciences, 554061) and streptavidin-Alexa Fluor 647 (Biolegend, 405237). Ovalbumin (Sigma, A5503-1G) was biotinylated using the EZ Sulfo-NHS-LC-Biotinylation kit according to the manufacturer's instructions (Thermo Scientific, A39257) and conjugated to streptavidin BV711 (BD Biosciences, 563262). Biotinylation was confirmed by ELISA prior to use in flow cytometry.

Single-Cell Sorting

PBMCs from sample 111 were enriched for B cells via positive selection using CD19 microbeads (Miltenyi Biotec, 130-050-301). PBMCs from all other donors were enriched for B cells by negative selection (Miltenyi Biotec, 130-101-638). All selection protocols were performed according to the manufacturer's instructions. Enriched B cells were incubated for 30 minutes on ice in FACS buffer (1× Phosphate-buffered saline (PBS), 2% calf serum, 1 mM EDTA) with fluorophore-labeled EDIII and ovalbumin, and in the presence of anti-human antibodies anti-CD3-APC-eFluro 780 (Invitrogen, 47-0037-41), anti-CD8-APC-eFluro 780 (Invitrogen, 47-0086-42), anti-CD14-APC-eFluro 780 (Invitrogen, 47-0149-42), anti-CD16-APC-eFluro 780 (Invitrogen, 47-0168-41), anti-CD20-PECy7 (BD Biosciences, 335793), and Zombie NIR (BioLegend, 423105). Single CD3⁻CD8⁻CD14⁻CD16⁻ZombieNIR⁻CD20⁺Ova⁻EDIII-PE⁺EDIII-AF647⁺ B cells were sorted using a FACS Aria III (Becton Dickinson) into individual wells of 96-well plates. Each well contained 4 L of a lysis buffer comprising 0.5×PBS, 10 mM DTT, and 3000 units/mL RNasin Ribonuclease Inhibitors (Promega, N2615). Sorted cells were snap-frozen on dry ice and then stored at −80° C. Antibody sequences are derived from memory B cells because they originate from small CD20⁺ cells, and the antibody genes were PCR-amplified using IgG-specific primers.

Antibody Sequencing, Cloning, and Expression

RNA from single cells was reverse transcribed using SuperScript III Reverse Transcriptase (Invitrogen, 18080-044). The resulting cDNA was stored at −20° C. until amplification of the variable IGH, IGL, and IGK genes by nested PCR followed by Sanger sequencing. Amplicons from the first PCR reaction were used as a template for nested PCR-amplification and Sequence- and Ligation-Independent Cloning (SLIC) into antibody expression vectors as previously described (Robbiani, D. F., et al., (2020) Nature 584, 437-442). Recombinant monoclonal antibodies were produced and purified as previously detailed (Klein, F., et al., (2014) J Exp Med 211, 2361-2372). The T036 F(ab) and F(ab′)2 were generated using the Pierce™ Fab Preparation kit (Thermo Scientific, 44988).

Plasmids for the Production of Reporter Virus Particle (RVP)

A West Nile virus subgenomic replicon-expressing plasmid encoding Renilla luciferase (pWNVII-Rep-REN-IB) and a ZIKV CprME expression plasmid had previously been obtained from Ted Pierson (NIH) (Pierson, T. C., et al., (2006) Virology 346, 53-65; Robbiani, D. F., et al., (2017) Cell 169, 597-609 e511). The ZIKV CprME expression plasmid was manipulated by restriction enzyme digestion and ligation to express the CprME of other flaviviruses as follows:

TBEV: synthetic DNA with CprME coding sequence (flanked at the 5′ by the polylinker and Kozak sequence GGAATTCGCGGCCGCCTCAGG (SEQ ID NO: 237) and at the 3′ by the stop codons and polylinker TAATAGTTAATTAACTCGAGCCGCGG (SEQ ID NO: 6667); “CprME-flanked”) corresponding to tick-borne encephalitis virus, Western European subtype strain Neudoerfl (GenBank NC_001672), was amplified with primers DFRp1532 (5-GGAATTCGCGGCCGCCTCAGG) (SEQ ID NO: 238) and DFRp1533 (5-GCGGCTCGAGTTAATTAA) (SEQ ID NO: 239) before cloning at the NotI and PacI sites of plasmid pPOWV-LB-CprME (see below), resulting in pTBEV-WE-CprME.

POWV-LB: synthetic DNA containing the CprME sequence (underlined) of POWV LB strain (GenBank: L06436.1 with 4 synonymous changes, in lowercase and bold, to reduce complexity;

(SEQ ID NO: 240)
CTACTTGGCAGTACATCTACGTATTAGTCATCGCTATTACCATGGTGAT
GCGGTTTTGGCAGTACATCAATGGGCGTGGATAGCGGTTTGACTCACGG
GGATTTCCAAGTCTCCACCCCATTGACGTCAATGGGAGTTTGTTTTGGC
ACCAAAATCAACGGGACTTTCCAAAATGTCGTAACAACTCCGCCCCATT
GACGCAAATGGGCGGTAGGCGTGTACGGTGGGAGGTCTATATAAGCAGA
GCTCTCTGGCTAACTAGAGAACCCACTGCTTACTGGCTTATCGAAATTA
ATACGACTCACTATAGGGAGACCCAAGCTGGCTAGTTAAGCTATCAACA
AGGAATTCGCGGCCGCCAGGCTATGATGACCACTTCTAAAGGAAAGGGG
GGCGGTCCCCCTAGGCGCAAGCTTAAAGTGACCGCAAATAAGTCGCGAC
CAGCAACGAGCCCAATGCCAAAGGGCTTCGTGCTGTCGCGCATGCTGGG
GATTCTTTGGCACGCCGTGACAGGCACGGCCAGACCCCCAGTGCTGAAA
ATGTTCTGGAAAACGGTACCACTGCGCCAGGCGGAGGCTGTTCTGAAGA
AGATAAAGAGAGTTATCGGGAACTTGATGCAGAGCCTTCACATGAGAGG
GCGTCGCAGGTCAGGTGTGGACTGGACTTGGATTTTTTTGACGATGGCG
TTGATGACCATGGCCATGGCAACCACCATCCACCGGGACAGGGAAGGAT
ACATGGTTATGCGGGCCAGTGGAAGGGACGCTGCAAGCCAGGTCAGGGT
ACAAAACGGAACGTGCGTCATCCTGGCAACAGACATGGGAGAGTGGTGT
GAAGATTCAATCACCTACTCTTGCGTCACGATTGACCAGGAGGAAGAAC
CCGTTGACGTGGACTGCTTCTGCCGAGGTGTTGATAGGGTTAAGTTAGA
GTATGGACGCTGTGGAAGGCAAGCTGGATCTAGGGGGAAAAGGTCTGTG
GTCATTCCAACACATGCACAAAAAGACATGGTCGGGCGAGGTCATGCAT
GGCTTAAAGGTGACAATATTCGAGATCATGTCACCCGAGTCGAGGGCTG
GATGTGGAAGAACAAGCTTCTAACTGCCGCCATTGTGGCCTTGGCTTGG
CTCATGGTTGATAGTTGGATGGCCAGAGTGACTGTCATCCTCTTGGCGT
TGAGTCTAGGGCCAGTGTACGCCACGAGGTGCACGCATCTTGAGAACAG
AGATTTTGTGACAGGAACTCAAGGGACCACCAGAGTGTCCCTAGTTTTG
GAACTTGGAGGCTGCGTGACCATCACAGCTGAGGGCAAGCCATCCATTG
ATGTATGGCTCGAAGACATTTTTCAGGAAAGCCCGGCTGAAACCAGAGA
ATACTGCCTGCACGCCAAATTGACCAACACAAAAGTGGAGGCTCGCTGT
CCAACCACTGGACCGGCGACACTTCCGGAGGAGCATCAGGCTAATATGG
TGTGCAAGAGAGACCAAAGCGACCGTGGATGGGGAAACCACTGtGGaTT
cTTcGGGAAGGGCAGTATAGTGGCTTGTGCAAAGTTTGAATGCGAGGAA
GCAAAAAAAGCTGTGGGCCACGTCTATGACTCCACAAAGATCACGTATG
TTGTCAAGGTTGAGCCCCACACAGGGGATTACTTGGCTGCAAATGAGAC
CAATTCAAACAGGAAATCAGCACAGTTTACGGTGGCATCCGAGAAAGTG
ATCCTGCGGCTCGGCGACTATGGAGATGTGTCGCTGACGTGTAAAGTGG
CAAGTGGGATTGATGTCGCCCAAACTGTGGTGATGTCACTCGACAGCAG
CAAGGACCACCTGCCTTCTGCATGGCAAGTGCACCGTGACTGGTTTGAG
GACTTGGCGCTGCCCTGGAAACACAAGGACAACCAAGATTGGAACAGTG
TGGAGAAACTTGTGGAATTTGGACCACCACATGCTGTGAAAATGGATGT
TTTCAATCTGGGGGACCAGACGGCTGTGCTGCTCAAATCACTGGCAGGA
GTTCCGCTGGCCAGTGTGGAGGGCCAGAAATACCACCTGAAAAGCGGCC
ATGTTACTTGTGATGTGGGACTGGAAAAGCTGAAACTGAAAGGCACAAC
CTACTCCATGTGTGACAAAGCAAAGTTCAAATGGAAGAGAGTTCCTGTG
GACAGCGGCCATGACACAGTAGTCATGGAGGTATCATACACAGGAAGCG
ACAAGCCATGTCGGATCCCGGTGCGGGCTGTGGCACATGGTGTCCCAGC
GGTTAATGTAGCCATGCTCATAACCCCCAATCCAACCATTGAAACAAAT
GGTGGCGGATTCATAGAAATGCAGCTGCCACCAGGGGATAACATCATCT
ATGTGGGAGACCTTAGCCAGCAGTGGTTTCAGAAAGGCAGTACCATTGG
TAGAATGTTTGAAAAAACCCGCAGGGGATTGGAAAGGCTCTCTGTGGTT
GGAGAACATGCATGGGACTTTGGCTCAGTAGGCGGGGTACTGTCTTCTG
TGGGGAAGGCAATCCACACGGTGCTGGGGGGAGCTTTCAACACCCTTTT
TGGTGGTGTTGGATTCATCCCTAAGATGCTGCTGGGGGTTGCTCTGGTC
TGGTTGGGACTAAATGCCAGGAATCCAACGATGTCCATGACGTTTCTTG
CTGTGGGGGCTTTGACACTGATGATGACAATGGGAGTTGGGGCATAATA
GTTAATTAACTCGAGCCGCGGTTCGAAGGTAAGCCT)

was PCR-amplified with primers DFRp1511 (5-ATCTACGTATTAGTCATCGCTATTA) (SEQ ID NO: 241) and DFRp1514 (5-ACCGCGGCTCGAGTTAATTAA) (SEQ ID NO: 242) and cloned at the Eco105I and SacII sites of plasmid pZIKV—HPF-CprME (Robbiani, D. F., et al., (2017) 169, 597-609 e511), resulting in pPOWV-LB-CprME.

POWV-DTV: A three-piece assembly PCR strategy was utilized. DNA upstream of the CMV promoter in pZIKV—HFP-CprME to just downstream of the beginning of the C-encoding region was PCR-amplified with primers RU-O-24611 (5′-CTTGACCGACAATTGCATGAAG-3′) (SEQ ID NO: 243) and RU-O-26690 (5′-CTTTCCTTTAGAAGTAGTCACCATAGCCTGCTTTTTTGTACAAAC-3′) (SEQ ID NO: 244), resulting in a fragment fusing the CMV promoter with POWV-DTV C-encoding sequences (bolded in primer RU-O-26690). Using as template DTVpl (Kenney, J. L., et al., (2018) Vector Borne Zoonotic Dis 18, 371-381), kindly provided by Aaron Brault and based on the Spooner strain of DTV, a fragment overlapping with the CMV promoter—DTV C fusion to the region just downstream of a SacII site within DTV genome was generated by PCR using oligos RU-O-26689

(SEQ ID NO: 245)
(5′-GTTTGTACAAAAAAGCAGGCTATGGTGACTACTTCTAAAGGAAA
G-3′)
and
RU-O-26711
(SEQ ID NO: 246)
(5′-GTTTCCCCATCCTCTATCGCTCTG-3′),

with bolded nucleotides indicating synonymous mutations introduced to ablate the SacII site. DNA was amplified using DTVpl as template and oligos RU-O-26710 (5′-CAGAGCGATAGAGGATGGGGAAAC-3′; (SEQ ID NO: 247) bolded nucleotides indicate synonymous mutations) and RU-O-26688 (5′-TTCGAACCGCGGCTGGGTCCTATTATGCTCCGACTCCCATTGTCATCATC-3′) (SEQ ID NO: 248) to generate a fragment overlapping the killed SacII site to the end of the envelope protein-coding region followed by a SacII site. The three DNA fragments were annealed, extended, and then PCR-amplified using primers RU-O-24611 and RU-O-26688. The resulting DNA fragment was digested with SnaBI and SacII and cloned into similarly digested pZIKV—HPF-CprME to generate pPOWV-DTV-CprME.

KFDV: synthetic DNA with the CprME-flanked sequence of Kyasanur fever disease virus, strain W-377 (GenBank JF416960.1), was amplified with primers DFRp1532 and DFRp1533 before cloning at the NotI and PacI sites of plasmid pPOWV-LB-CprME (see above), resulting in pKFDV-W-377-CprME.

LGTV: the CprME of Langat virus, isolate TP21-636, was amplified from a plasmid kindly provided by Dr. Sonja Best (Rocky Mountain Laboratories of NIH/NIAID) with primers DFRp1563 (5-GGAATTCGCGGCCGCCTCAGGATGGCCGGGAAGGCCGTTCTA) (SEQ ID NO: 249) and DFRp1566 (5-CCGCGGCTCGAGTTAATTAACTATTAGGCTCCAACCCCCAGAGTCAT) (SEQ ID NO: 250) before cloning at the NotI and PacI sites of plasmid pPOWV-LB-CprME, resulting in pLGTV-TP21-636-CprME. Two nucleotide mutations from GenBankNC_003690 (A590G and A1893C).

LIV: synthetic DNA with the CprME-flanked sequence of louping ill virus, isolate LI3/1 (GenBank KP144331), was amplified with primers DFRp1532 and DFRp1533 before cloning at the NotI and PacI sites of plasmid pPOWV-LB-CprME, resulting in pLIV-LI3/1-CprME.

OHFV: synthetic DNA with the CprME-flanked sequence of Omsk hemorrhagic fever virus, strain Bogoluvovska (GenBank NC_005062), was amplified with primers DFRp1532 and DFRp1533 before cloning at the NotI and PacI sites of plasmid pPOWV-LB-CprME, resulting in pOHFV-CprME.

To confirm the absence of PCR-induced errors, all PCR-derived regions were sequenced in the final plasmids.

RVP Production

RVPs were produced by co-transfecting 1 μg of pWNVII-Rep-REN-IB plasmid with 3 μg of the flavivirus CprME plasmid of choice into the permissive cell line Lenti-X 293T, using Lipofectamine 2000 (Invitrogen, 1166803) according to the manufacturer's instructions. Cells were seeded 24 hours previously at 1×10⁶ cells/well in collagen-coated 6-well plates. Following transfection and 6 hours incubation at 37° C., excess DNA-lipid complexes were removed by aspiration, and the media was replaced with DMEM (Gibco) containing 20 mM HEPES and 10% FBS. For the next 72 hours, in 24-hour intervals, RVP-containing supernatants were harvested, filtered through a 0.45 micron filter and frozen at −80° C., and media replaced with DMEM containing 20 mM HEPES and 10% FBS. Frozen RVPs were later thawed and titrated on Huh-7.5 cells to determine the dilution of RVPs at which cells express 1×10⁶ RLU in the absence of sera or antibody.

RVP Neutralization Assays

96-well plates were seeded with 7,500 Huh-7.5 cells/well in 50 μL of DMEM (Gibco) supplemented with 10% FBS and 1% non-essential amino acids (NEAA). After 24 hours, 100 μL of diluted RVPs were combined with 100 μL of diluted sera or antibody, incubated for 1 hour at 37° C., and then 50 μL of the mix was added in triplicate to the plated cells. RVPs are diluted appropriately in BA-1 diluent (Medium 199 (Lonza) supplemented with 1% BSA and 100 units/mL Penicillin/Streptomycin) to achieve the desired RLU expression. After an additional 24 hours of incubation at 37° C., media was aspirated off the cells, replaced with 35 μL of lysis buffer (Promega, E2810), and the plates were frozen at −80° C. 15 μL of the subsequently thawed lysis buffer was used for Renilla luciferase expression measurement, using the Renilla Luciferase Assay System (Promega, E2810). Sera were either diluted to 1:600,000 final concentration for TBEV RVP neutralization screening or serially diluted to generate curves. Recombinant monoclonal antibodies were used at 10 g/mL final concentration and serially diluted 1:3 for neutralization assays. The half-maximal neutralization titer (NT₅₀) and the half-maximal inhibitory concentration (IC₅₀) were determined by non-linear regression analysis using Prism software (GraphPad). In the cross-neutralization screening against the panel of flavivirus RVPs, recombinant antibodies were assayed at 1 μg/mL final concentration using the protocol described above, and the results compared to no antibody control. In experiments using antibody fragments, equimolar concentrations of antibody fragments and immunoglobins were used. In RVP experiments using antibody combinations, T036 was used at 1 g/mL, and 4G2 (clone D1-4G2-4-15; Sigma cat. MAB10216) at 10 μg/mL final concentration.

ELISA Assays

Binding of serum IgG or recombinant IgG antibodies to EDIII proteins was measured by standard ELISA. High-binding 96 well plates (Costar, 07-200-721) were coated overnight with 250 ng of the EDIII protein in PBS per well at room temperature; plates were then blocked with 0.1 mM EDTA, 0.05% Tween, and 2% BSA in PBS for 2 hours at room temperature. Samples were diluted in PBS-T, added to plates, and incubated for an additional 1 hour at room temperature. Secondary goat anti-human-IgG F(ab′)2 fragments conjugated to HRP (Jackson Immunoresearch, 109-036-088) were diluted 1:5,000 in PBS-T, added to the plates, and incubated again for 1 hour at room temperature. Between each step, the plates were washed with PBS-T four times. Plates were finally developed using TMB substrate (ThermoScientific, 34021); the reaction was stopped using 1M sulfuric acid, and the plates were read at 450 nm. Sera were screened for binding at 1:500 dilution. Recombinant monoclonal antibodies were diluted to 10 g/mL and serially diluted 1:3; the half effective concentration (EC₅₀) was determined by non-linear regression analysis using Prism 8 (GraphPad). For cross-binding assays, recombinant antibodies were assayed at 1 g/mL according to the protocol described above using the panel of flavivirus EDIII proteins. The anti-HIV monoclonal antibody 10-1074 was used as isotype control (Mouquet, H., et al., (2012) Proc Natl Acad Sci USA 109, E3268-3277). Antibodies with optical density>2.5 times isotype control signal were considered cross-reactive. The TBEV clinical tests (Tables 1A and 1B) were conducted using the EIA TBE Virus IgG (TBG096) and EIA TBE Virus IgM (TBM096) kits from TestLine Clinical Diagnostics.

Viruses and Cells

The low-passage TBEV strain Hypr was provided by the Collection of Arboviruses, Institute of Parasitology, Biology Centre of the Czech Academy of Sciences, Ceske Budejovice, Czech Republic (http://www.arboviruscollection.cz/index.php?lang=en). The virus was originally isolated from the blood of a diseased 10-year-old child in Brno, Czech Republic (former Czechoslovakia), in 1953. The low-passage TBEV strain Neudoerfl was kindly provided by Professor F. X. Heinz, Medical University in Vienna, Austria. The virus was originally isolated from the tick Ixodes ricinus in Austria in 1971. Prior to their use in in vitro experiments, virus strains were propagated in suckling mouse brains and/or BHK-21 cells.

PS cells (porcine kidney stable) (Kozuch, O. and Mayer, V., (1975) Acta Virol 19, 498) were cultured at 37° C. in Leibovitz (L-15) medium supplemented with 3% fetal bovine serum, 100 U/mL penicillin, 100 μg/mL streptomycin, and 1% L-glutamine (Sigma-Aldrich, Prague, Czech Republic).

Plaque Assay

To determine virus titer in cell culture supernatants, plaque assays were performed as previously described (De Madrid, A. T., and Porterfield, J. S. (1969) Bull World Health Organ 40, 113-121) with slight modifications (Formanova, P. P., et al., (2019) J Neuroinflammation 16, 205). Briefly, 10-fold dilutions of virus plus a suspension of PS cells (1.3×10⁵ cells per well) were added to 24-well tissue culture plates. After 4 hours of incubation at 37° C. with 0.5% CO₂, each well was overlaid with carboxymethylcellulose (1.5% in L-15 medium). After a 5-day incubation at 37° C. and 0.5% CO₂, the cell monolayers were visualized using naphthalene black. Viral titers were expressed as plaque-forming units (pfu) per milliliter.

Virus Neutralization Test (VNT)

VNT was performed as described previously (Sirmarovi, J., et al., (2014) Ticks Tick Borne Dis 5, 523-527) with several modifications. Briefly, monoclonal antibodies (T025, T028, T034, and T038) were diluted to 2.5 μg/ml in L-15 medium and then serially diluted 1:2 in 96-well plates. Diluted monoclonals were incubated with 50 pfu per well of TBEV-Hypr (sufficient to cause 90-95% cytolysis) for 90 min at 37° C. Thereafter, 5×10⁴ PS cells were added per well. After 4 days incubation at 37° C., the cytopathic effect (CPE) was monitored microscopically, and cell viability was measured using the Cell Counting Kit-8 (Dojindo Molecular Technologies, Inc., Munich, Germany) according to the manufacturer's instructions. Half-maximal inhibitory concentration (IC₅₀) was calculated from two independent experiments done in octuplicates using GraphPad Prism (version 7.04, GraphPad Software, San Diego, CA, USA).

Effect of Antibodies on Virus Growth

Monoclonal antibodies (T036, T038, and 10-1074) were diluted to 0.5 or 0.05 μg/ml in L-15 medium and incubated with TBEV-Hypr (50 pfu/well) or TBEV-Neudoerfl (500 pfu/well or 2,500 pfu/well) in 96-well plates for 90 min at 37° C. After incubation, 5×10⁴ PS cells were added per well. After 24 and 48 hours of incubation at 37° C. and 0.5% CO₂, culture media were harvested, and virus titer was determined by plaque assay as described above, and the cell monolayers were fixed with cold acetone-methanol (1:1), blocked with 10% fetal bovine serum, and incubated with mouse anti-flavivirus antibody (1:250 dilution, clone D1-4G2-4-15; Sigma cat. MAB10216), as described previously (Stefanik, M., et al., (2020) Microorganisms 8). After washing, the cells were labeled with secondary goat anti-mouse antibody conjugated to fluorescein isothiocyanate (FITC; diluted 1:500, Sigma cat. AP181F) and counterstained with 4′,6-diamidino-2-phenylindole (DAPI, diluted to 1 μg/mL) to visualize cell nuclei. The fluorescence signal was recorded with an Olympus IX71 epifluorescence microscope and processed by ImageJ software.

Virus-Cell Binding Assay

TBEV (strain Hypr; 100 PFU) was pre-incubated with monoclonal antibodies or antibodies in combination (T036 and 10-1074 were used at 0.5 μg/ml, and D1-4G2-4-15 [4G2] was used at 10 μg/ml final concentration) in L-15 medium supplemented with 3% fetal bovine serum, 100 U/mL penicillin, 100 μg/mL streptomycin, and 1% L-glutamine (Sigma-Aldrich, Prague, Czech Republic) for 1.5 hours at 37° C. TBEV-antibody complexes were then added to pre-chilled confluent PS cell monolayers in 6-well plates (1 mL per well). After 1 hour at 4° C., the inoculum was removed, and cells were washed three times with PBS to remove unbound virus. L-15 medium supplemented with 3% fetal bovine serum, 100 U/mL penicillin, 100 μg/mL streptomycin, and 1% L-glutamine and 1.5% of CMC was added (4.5 ml per well), and the temperature was shifted to 37° C. to allow infection of the cells. After 5 days of incubation at 37° C. and 0.5% CO₂, the cell monolayers were stained using naphthalene black, and the plaques were visualized and counted to determine the number of viral particles that bound to the cells during the incubation step.

Statistical Analyses

For all live virus experiments, ANOVA followed by Tukey's multiple comparison tests and Student's t-tests were performed with log-transformed data. GraphPad Prism (version 7.04, GraphPad Software, San Diego, CA, USA) was used for analysis. Otherwise, data were analyzed using Mann-Whitney tests or ANOVA and Tukey's multiple comparison tests as specified, and comparison of survival curves was analyzed by Log-rank (Mantel-Cox) test, calculated in GraphPad Prism (version 8.4.3, GraphPad Software, San Diego, CA, USA). p values<0.05 were considered significant.

Crystallization, Structure Determination, and Refinement

Complexes for crystallization were produced by mixing Fab and antigen at a 1:1 molar ratio and incubating at room temperature for 1-2 hours. Crystals of T025 Fab-TBEV-WE EDIII-His-Avitag complex (space group P2₁; a=55.5 Å, b=66.7 Å, c=91.2 Å, α=90°, β=94.6°, γ=90°; one molecule per asymmetric unit) were obtained by combining 0.2 μL of crystallization complex with 0.2 μL of 0.1M sodium citrate tribasic dihydrate pH 5.0, 10% PEG 6000 in sitting drops at 22° C. Crystals of T025 Fab-TBEV-FE EDIII-His-Avitag complex (space group P2₁2₁2; a=56.96 Å, b=69.72 Å, c=180.20 Å, α=90°, β=90°, γ=90°; one molecule per asymmetric unit) were obtained by combining 0.2 μL of crystallization complex with 0.2 μL of 0.1M sodium citrate tribasic dihydrate pH 5.0, 10% PEG 6000 in sitting drops at 22° C. Crystals of T025 Fab-TBEV-Si EDIII complex (space group P2₁; a=55.4 Å, b=67.2 Å, c=91.2 Å, α=90°, β=94.8°, γ=90°; one molecule per asymmetric unit) were obtained by combining 0.2 μL of crystallization complex with 0.2 μL of 5% (+/−)-2-Methyl-2,4-pentanediol, 0.1M HEPES pH 7.5, 10% PEG 10,000 in sitting drops at 22° C. Crystals were cryoprotected with 25% glycerol. Crystals of T036 Fab-LIV EDIII complex (space group P1; a=67.0 Å, b=67.9 Å, c=82.4 Å, α=88.0°, β=73.2°, γ=70.6°; two molecules per asymmetric unit) were obtained by combining 0.2 μL of crystallization complex with 0.2 μL of 0.15M Lithium Sulfate monohydrate, 0.1M Citric acid pH 3.5, 18% PEG 6,000 in sitting drops at 22° C. Crystals were cryoprotected stepwise to 25% glycerol before being cryopreserved in liquid nitrogen.

X-ray diffraction data (Table 6) were collected at Stanford Synchrotron Radiation Lightsource (SSRL) beamline 12-2 using a Dectris Pilatus 6M detector. The data were integrated using Mosfim (Battye, T. G., et al., (2011) Acta Crystallogr D Biol Crystallogr 67, 271-281) and scaled using CCP4 (Winn, M. D., et al., (2011) Acta Crystallogr D Biol Crystallogr 67, 235-242). Four 1800 datasets from the same crystal were collected using different detector distances for the T036-LIV crystals and then merged and scaled using CCP4. The T025-TBEV-WE EDIII complex structure was solved by molecular replacement using the V_{H}V_{L} domains from PDB 2GHW, the C_{H}C_{L} domains from PDB 4OGX, and TBEV EDIII from PDB 6J5F as search models in PHASER (McCoy, A., et al., (2007) J Appl Crystallogr 40, 658-674). The model was refined to 2.24 Å resolution using an iterative approach involving refinement in Phenix (Adams, P. D., et al., (2010) Acta Crystallogr D Biol Crystallogr 66, 213-221) and manual rebuilding into a simulated annealed composite omit map using Coot (Emsley, P., and Cowtan, K., (2004) Acta Crystallogr D Biol Crystallogr 60, 2126-2132). Residues that were disordered and not included in the model were HC residues 214-219 and the 6× His tag (SEQ ID NO: 6668); residue 214 of the LC; and residues 299-302, 397, and the 6× His-tag (SEQ ID NO: 6668) and Avi-tag of the TBEV-WE EDIII domain. The T025-TBEV-FE EDIII and T025-TBEV-Si EDIII complex structures were solved similarly using the partially-refined T025-TBEV-WE EDIII structure as the molecular replacement model. The T025-TBEV-FE EDIII model was refined to 2.35 Å resolution, and the T025-TBEV-Si EDIII model was refined to 1.86 Å resolution using the iterative approach described for T025-TBEV-WE EDIII. The T036-LIV EDIII complex structure was solved by molecular replacement using the V_{H}V_{L} domains from PDB 40B5, the C_{H}C_{L} domains from the partially-refined T025-TBEV-WE EDIII structure, and TBEV EDIII from PDB 6J5F as search models in PHASER (McCoy, A., et al., (2007) J Appl Crystallogr 40, 658-674). The model was refined to 2.4 Å using an iterative strategy as described above that included non-crystallographic symmetry restraints during the initial stages of refinement. Residues that were disordered and not included in the model were HC residues 128-133, 188-190 (chain A), 214-219, and the 6× His tag (SEQ ID NO: 6668); residues 212-214 (chain L) or 213-214 (chain B) of the LC; and residues 301 and 397 (chain C) of the LIV EDIII domain. In addition, there was extra density present in a simulated annealed omit map (Phenix) near residues E98_{HC} and S94_{LC} in both copies of the Fab in the asymmetric unit that were not modeled. The Kabat numbering scheme was used for Fab numbering. Structures were superimposed, RMSDs were calculated, and figures were generated using PyMOL. Buried surface areas and hydrogen bonds were determined using PDBePISA (Krissinel, E., and Henrick, K., (2007) J Mol Biol 372, 774-797). Fab-antigen contact residues were identified as residues in which any atom is within 4 Å of an atom on the other protein. The distance and geometry criteria used for assigning hydrogen bonds were a distance of <4.0 Å and a hydrogen bond angle of 90-270°. The maximum distance allowed for a van der Waals interaction was 4.0 Å.

Animal Ethics Statement

The research complied with all relevant European Union guidelines for work with animals and was in accordance with Czech national law guidelines on the use of experimental animals and protection of animals against cruelty (Animal Welfare Act No. 246/1992 Coll.). The protocol was approved by the Committee on the Ethics of Animal Experimentation of the Institute of Parasitology and of the Departmental Expert Committee for the Approval of Projects of Experiments on Animals of the Czech Academy of Sciences (permit No. 4253/2019).

Mice and Virus Inoculation

Specific pathogen-free BALB/c mice were obtained from ENVIGO RMS B.V. (Horst, the Netherlands). Sterilized pellet diet and water were supplied ad libitum. In all experiments, female mice aged 6-8 weeks were used. Mice were housed in individually ventilated plastic cages (Techniplast) with wood-chip bedding, with a constant temperature of 22° C., a relative humidity of 65%, and under a 12 hr light/dark cycle. Three mice per group were used in experiments. Mice were inoculated intraperitoneally one day prior to or one day post infection with monoclonal antibodies T025 or 10-1074 in 200 ul PBS, and infected subcutaneously with 100 pfu of TBEV-Hypr (propagated 8 times in suckling mouse brains). Mice were monitored for symptoms and survival over time and euthanized when reaching a humane endpoint.

### Example 2

Serological Responses in a TBEV-Infected Cohort

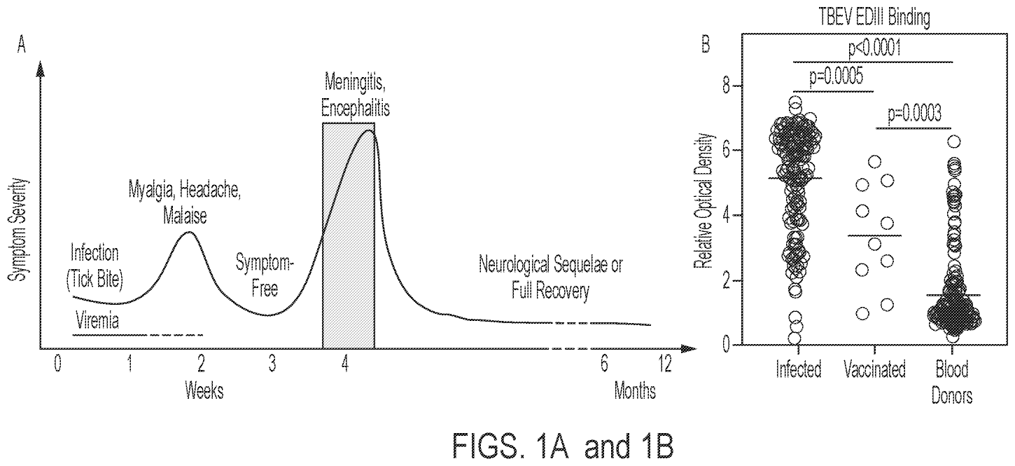

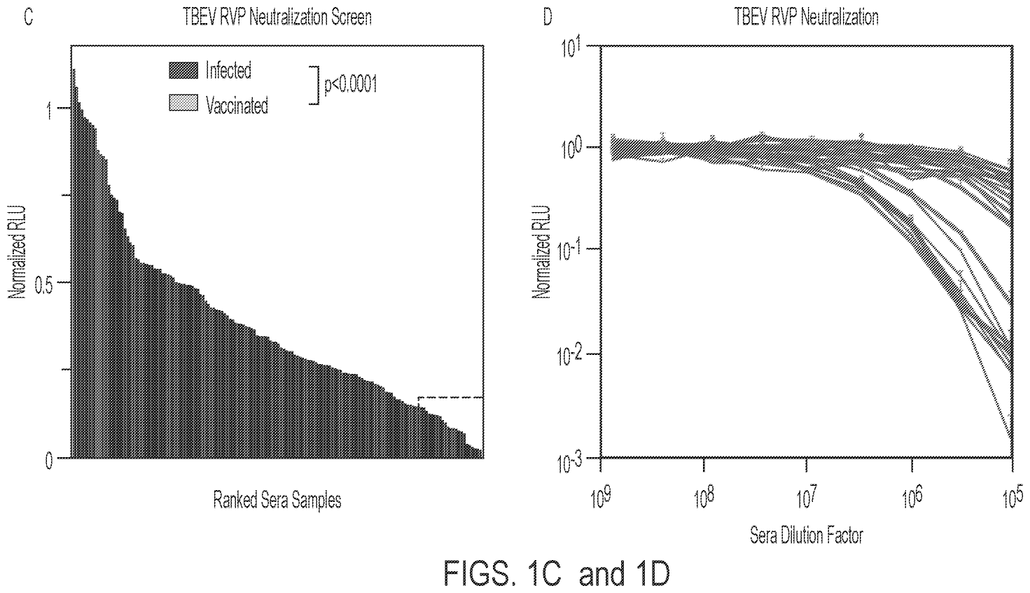

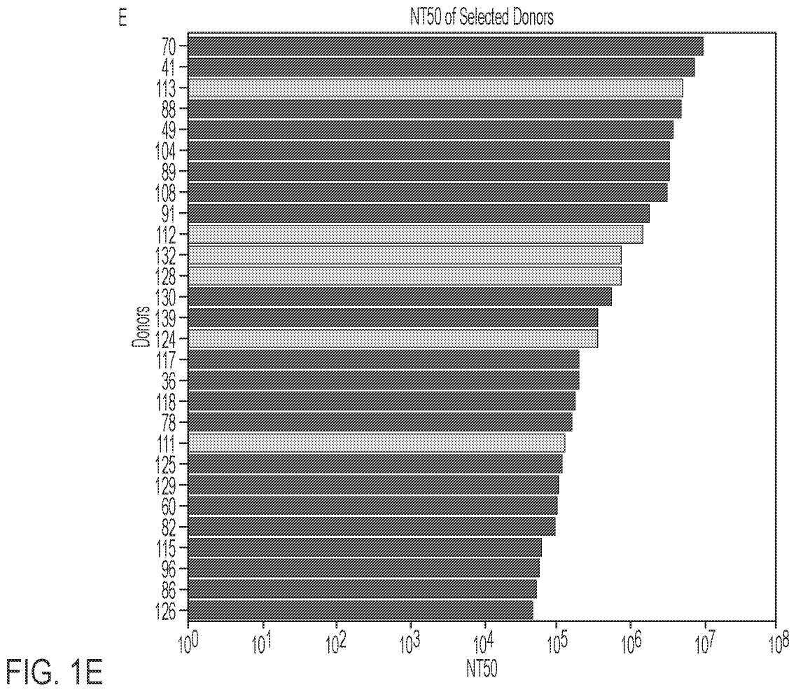

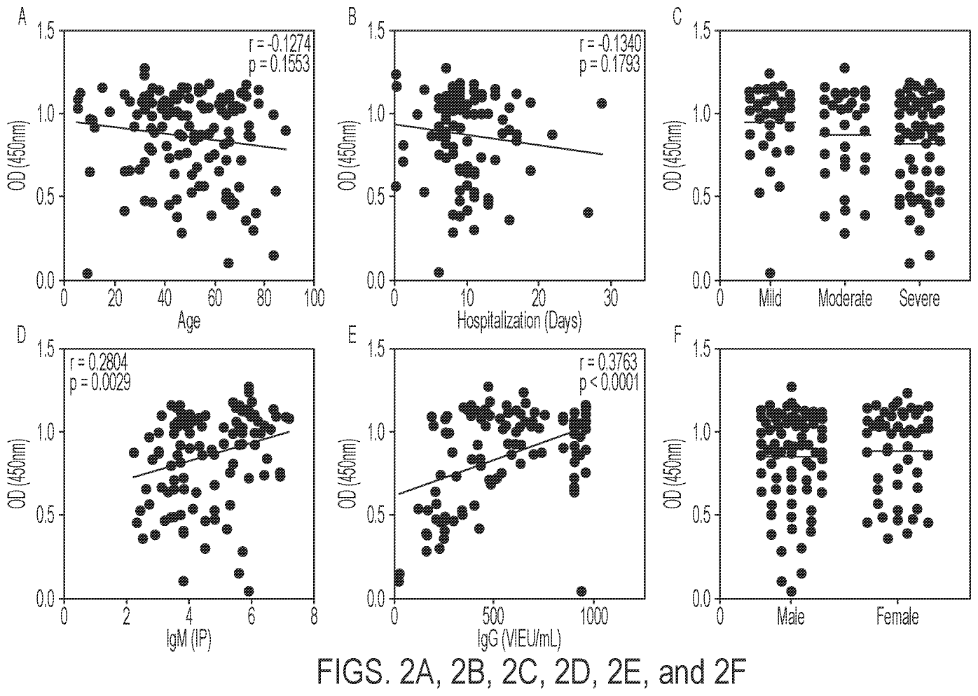

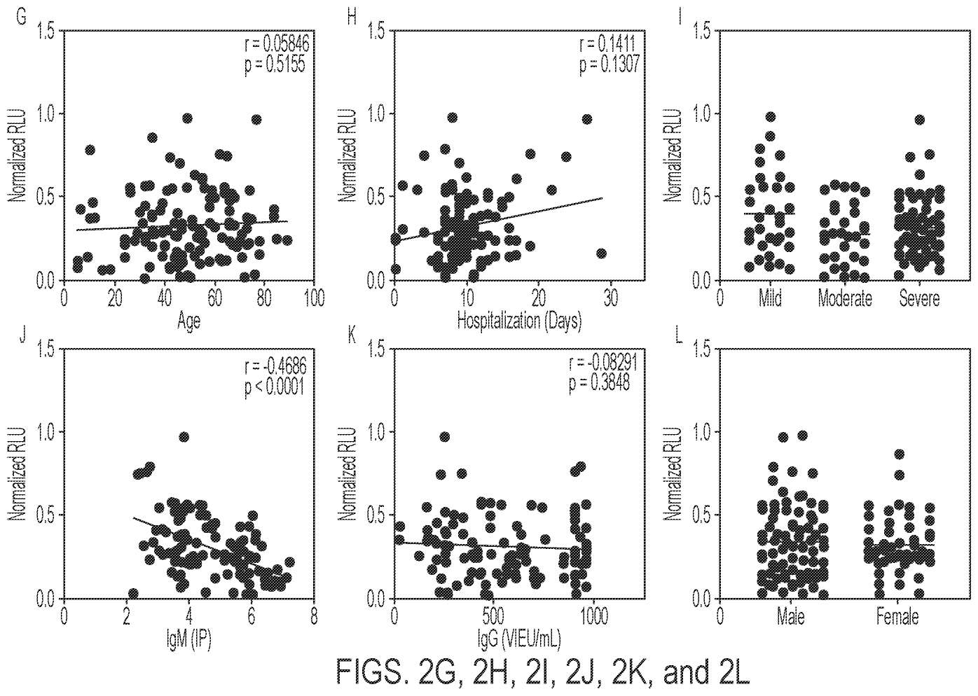

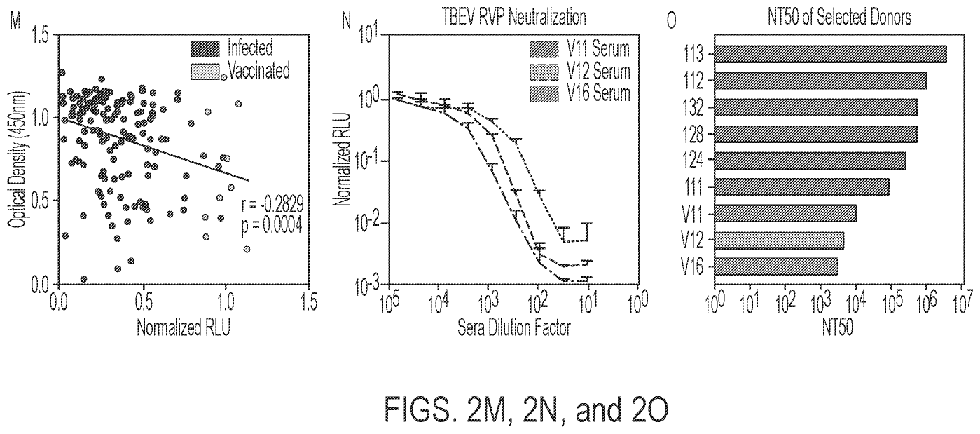

Sera from 141 individuals hospitalized with TBE during the 2011 and 2018 outbreaks in the Czech Republic were analyzed. Samples were obtained at the time of hospitalization, during the encephalitic phase of disease (FIG. 1A) (Holzmann, H., (2003) Vaccine 21, S36-S40). In agreement with previous reports (Bogovič, P., et al., (2018) Travel Med Infect Dis 26, 25-31; Bogovic, P., and Strle, F., (2015) World J Clin Cases 3 430-441), the cohort was characterized by higher incidence in males (61.1%) and older individuals (mean age=49 years; Tables 1A and 1). Control sera were also obtained from 168 randomly selected blood bank donors and from 10 individuals vaccinated against TBEV (Tables 1A and 1). All sera were screened by ELISA at a dilution of 1:500 for the presence of IgG antibodies binding to the EDIII of TBEV (FIG. 1). The signal in infected individuals was significantly higher than in the vaccinated and blood donor groups (p=0.0159 and p<0.0001 by ANOVA using Tukey's correction, respectively; FIG. 1). There was no correlation between TBEV EDIII ELISA reactivity and age or duration of hospitalization (FIG. 2A-O).

To evaluate serum neutralizing activity, samples obtained from recovered and vaccinated individuals were screened at a 1:6×10⁵ dilution for neutralization using luciferase-expressing TBEV reporter virus particles (RVPs; see Methods) (Pierson, T. C., et al., (2006) Virology 346 53-65). Neutralizing activity ranged from complete to undetectable, was significantly lower in vaccinees (p<0.0001; FIG. 1C) and correlated with EDIII binding in ELISA (p=0.0004; FIG. 2M). The half-maximal neutralizing titers (NT₅₀) for the top 28 infected individuals varied from 0.37-6.7×10⁶ (FIGS. 1D-E). In contrast, vaccinees showed NT₅₀s of 0.32-1.0×10⁴ (FIGS. 2N-0). It was concluded that individuals hospitalized for TBEV infection show a broad distribution of EDIII binding and neutralizing activity that is generally higher than the vaccinees in this cohort.

B Cell Memory Converges on Specific Antibody Genes

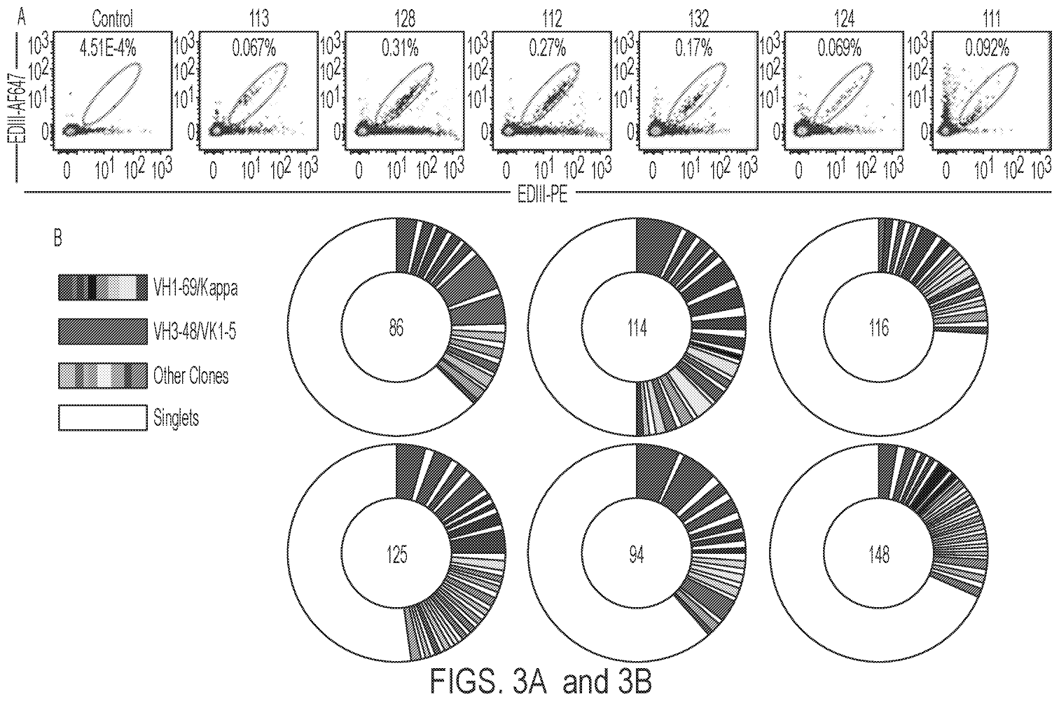

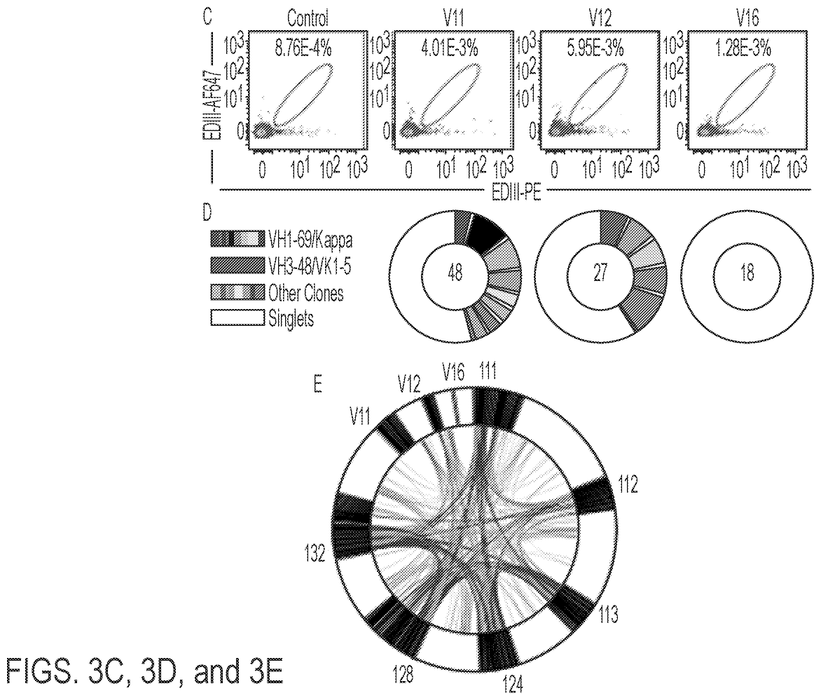

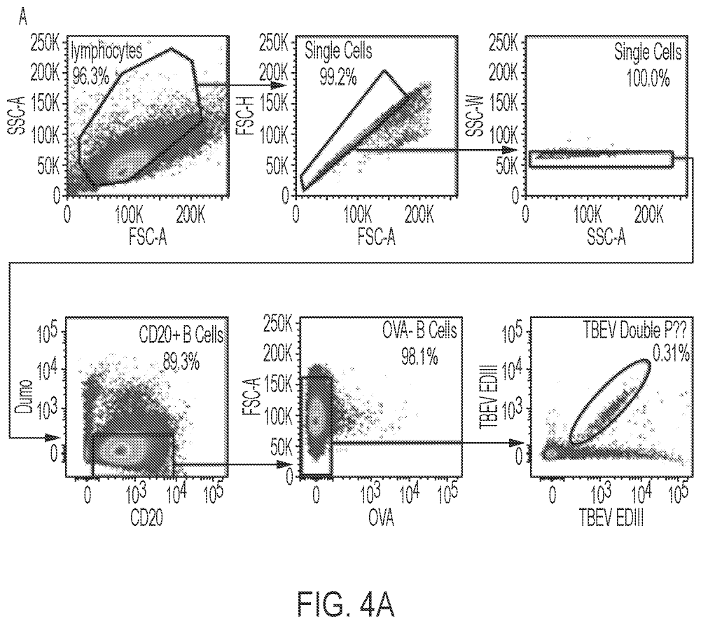

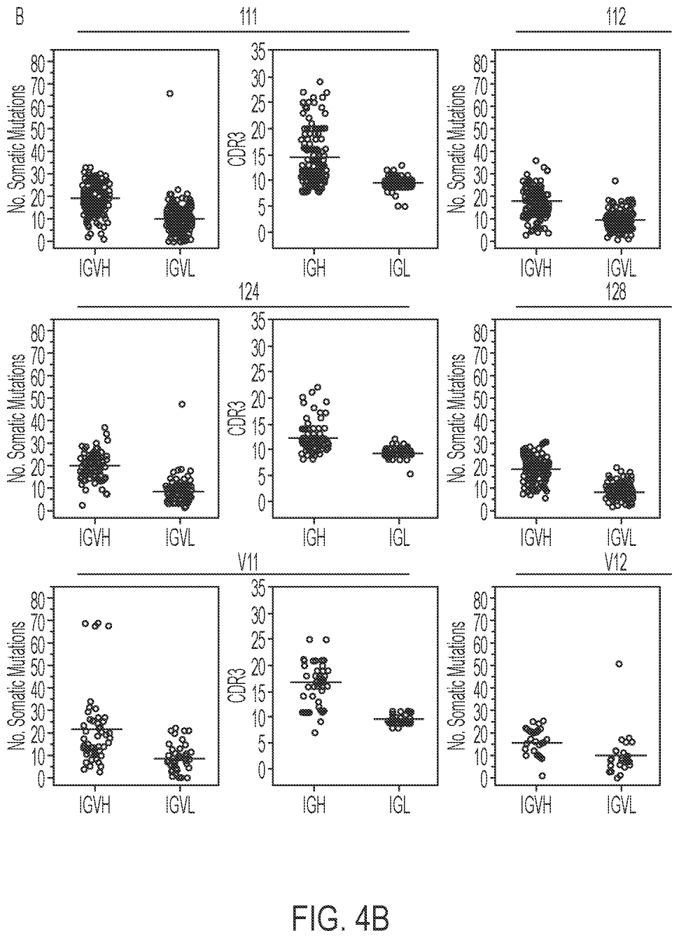

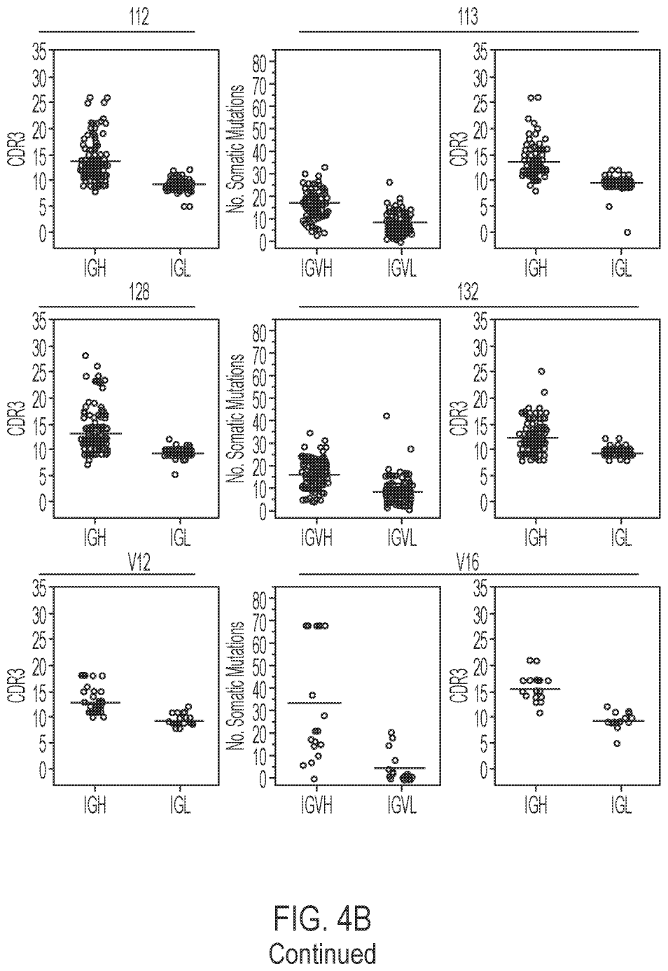

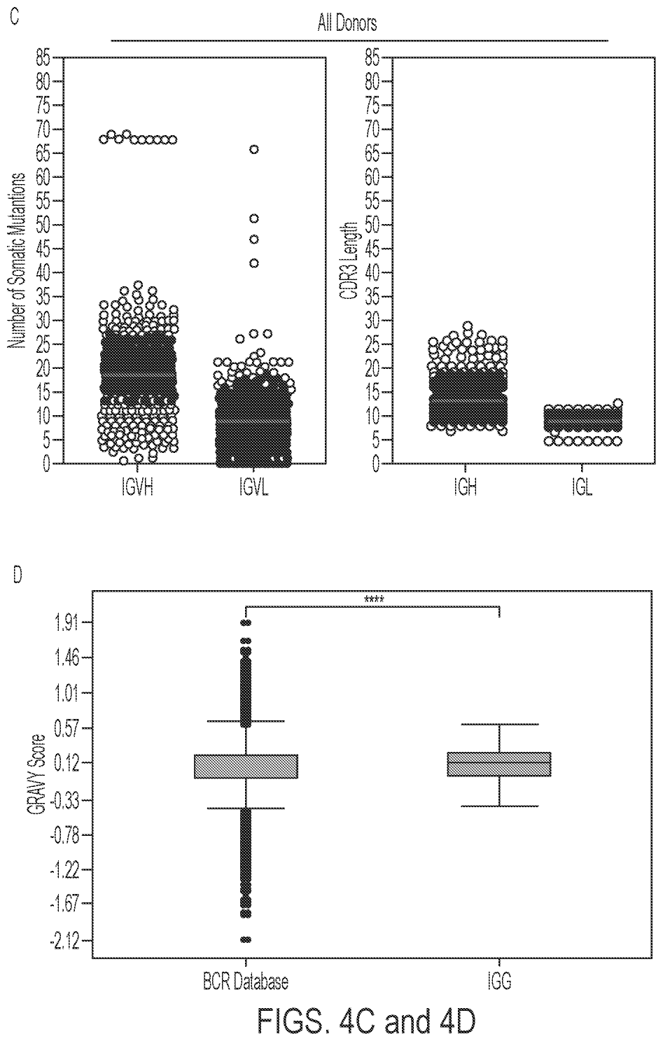

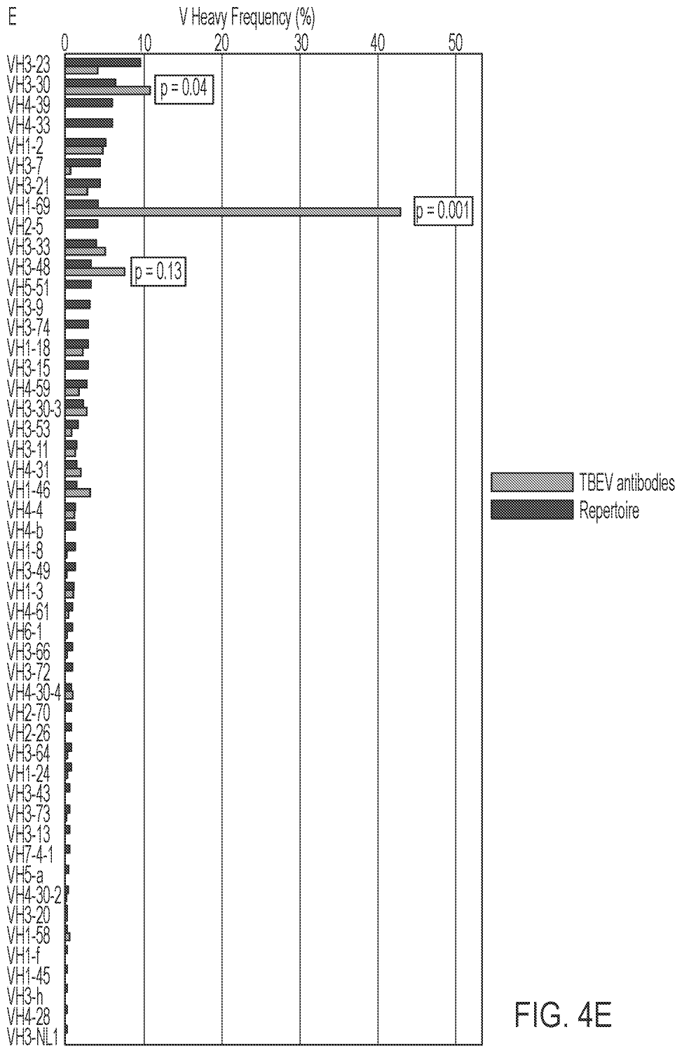

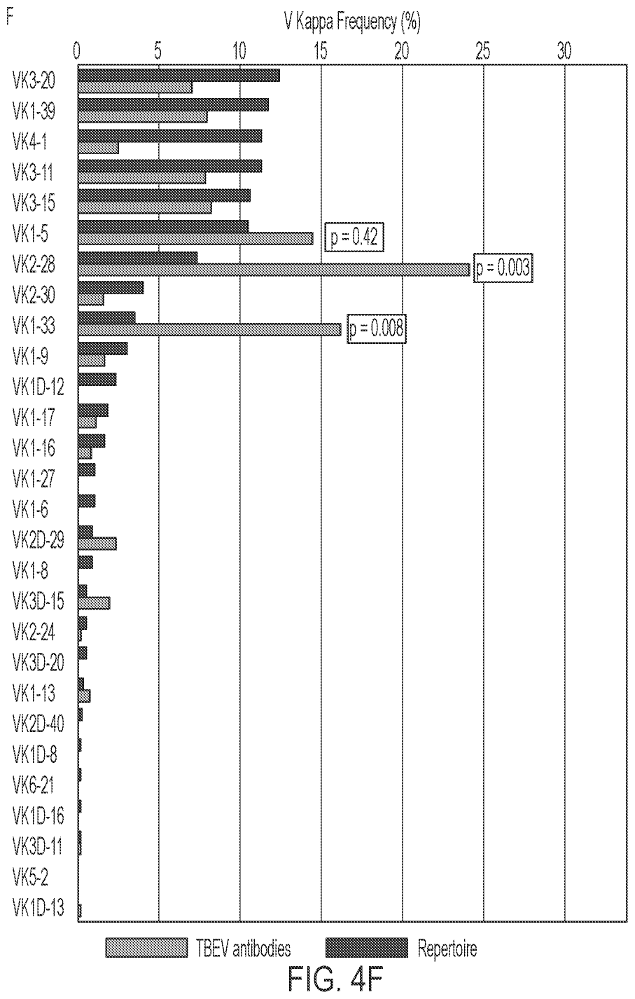

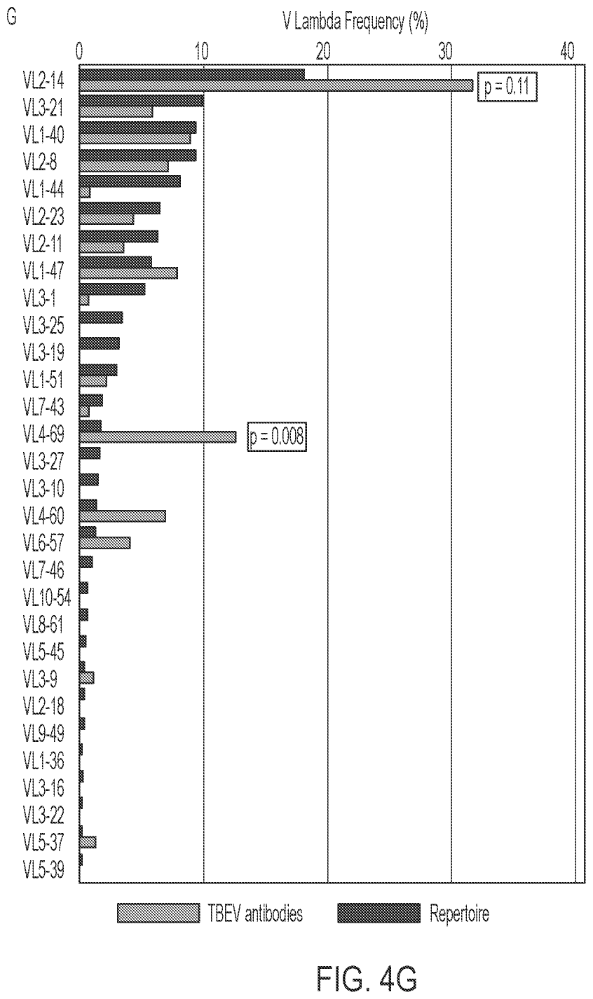

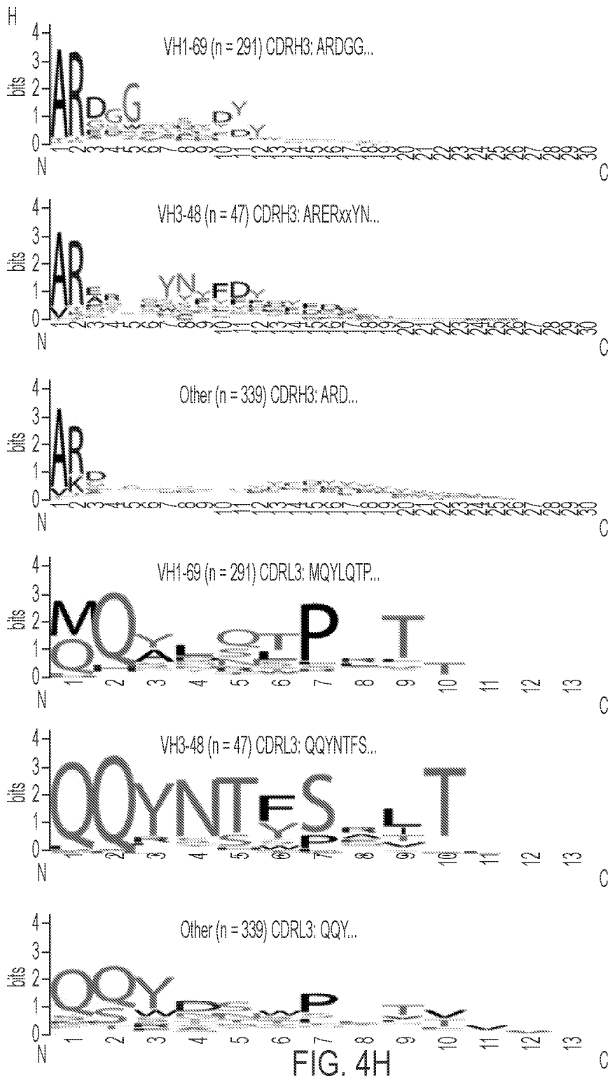

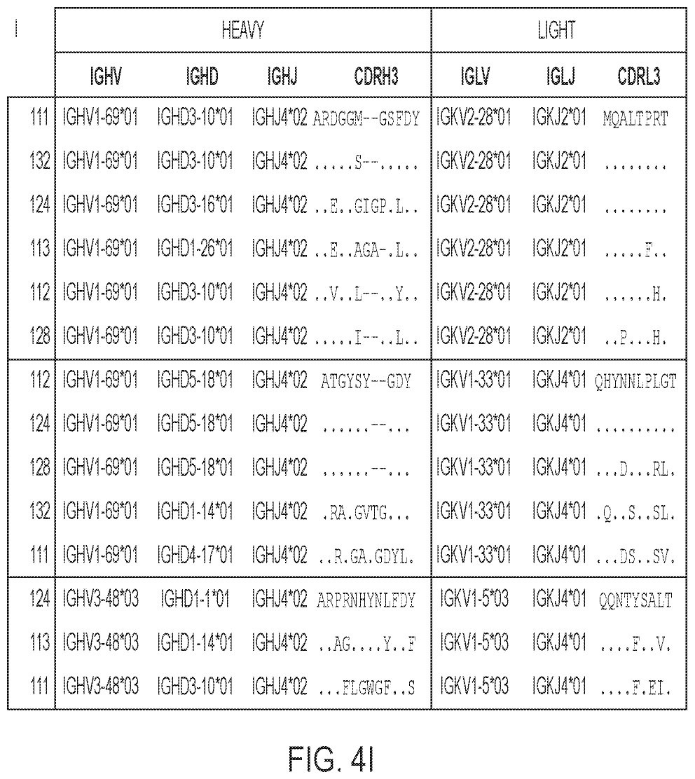

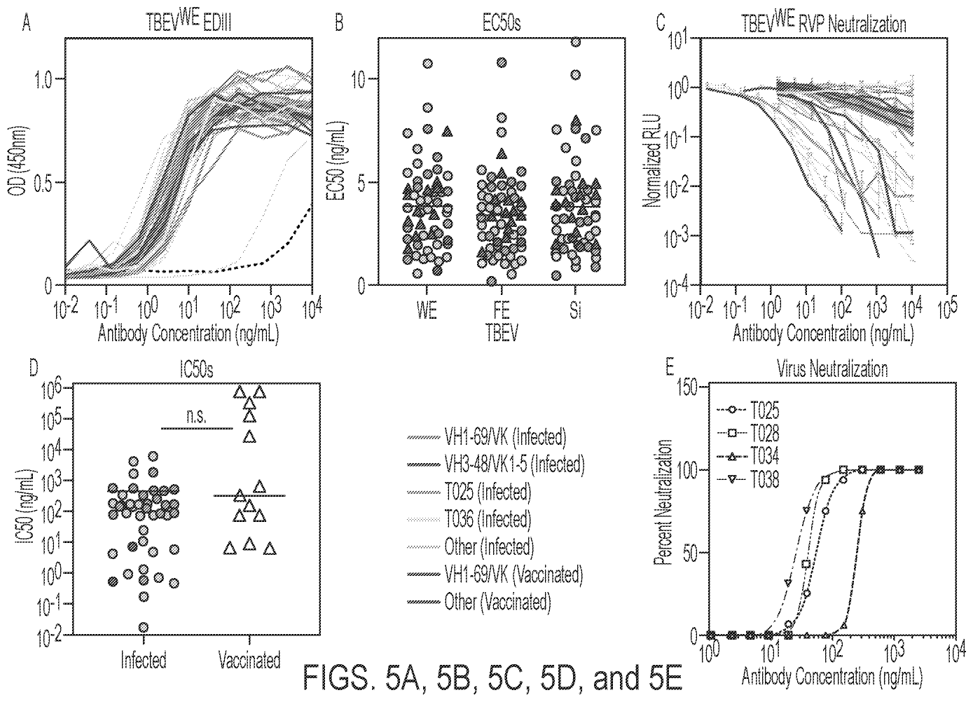

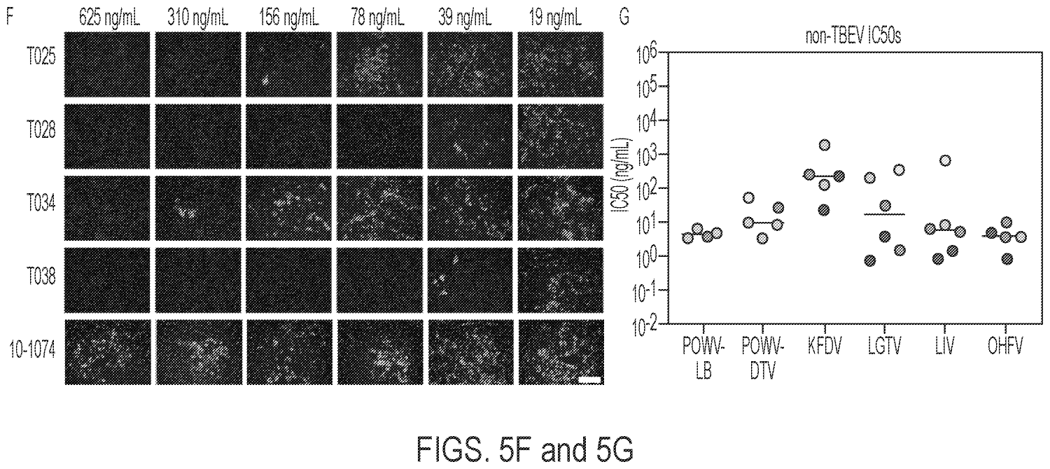

To characterize the anti-TBEV antibodies, TBEV-specific B cells from peripheral blood of six infected individuals (orange in FIGS. 1D and 1E) and 3 vaccinees were purified (FIGS. 3A-D and 4A). The frequency of TBEV EDIII-specific B cells among circulating CD20⁺ B cells was higher in the infected group (0.067-0.31%) compared to vaccinees (1.28-5.95×10⁻³%). In total, 776 IgG antibody heavy and light chain gene pairs were amplified by RT-PCR and sequenced (EXAMPLE 1, FIGS. 3B and 5D, and Tables 2A-2J). The average somatic hypermutation in IGVH and IGVL was 18 and 9 nucleotides, respectively, CDR3 length was normal (mean CDRH3 length of 13.5 and mean CDRL3 length of 9.4), and hydrophobicity was slightly increased compared to control (p<0.0001; FIGS. 4B-D) (Briney, B., et al., (2019) Nature 566, 393-397; Rock, E. P., et al., (1994) J Exp Med 179, 323-328). As with other viral pathogens including HIV-1, Zika, hepatitis B, and SARS-CoV-2 (Robbiani, D. F., et al., (2017) Cell 169, 597-609 e511; Robbiani, D. F., et al., (2020) Nature 584, 437-442; Scheid, J. F., et al., (2011) Science (New York, NY) 333, 1633-1637; Wang, Q., et al., (2020) Cell Host Microbe 28, 335-349.e336; West, A. P., et al., (2012) Proc Natl Acad Sci USA 109, E2083-20990), many of the sequences were found in expanded B cell clones (37.9%, FIGS. 3B and D).

Sequence analysis revealed antibodies with similar features within and between individuals (FIGS. 3B, 3D and 3E, Tables 2A-2J and 3). For example, VH1-69 and VH3-48 accounted for 59.2% and 7.5% of all clonal sequences, respectively (shades of blue and red in FIGS. 3B and 3D). In addition, related sequences containing these VH genes were found in multiple donors (purple lines in FIG. 3E). Usage of VH1-69, VK2-28, VK1-33, and V_{L}4-69 genes in infected donors was significantly over-represented (p<0.01); VH3-48, VK1-5, and V_{L}2-14 genes were also enriched, although not significantly (FIGS. 4E-G). In some cases, including clonally expanded IGVH1-69/IGVK2-28 and IGVH3-48/IGVK1-5 antibodies, sequence similarities between individual donors extended to the IGH and IGL CDR3s (Table 3, FIGS. 4H and 4I). It was concluded that the memory B cell response to the TBEV EDIII converges towards specific antibody genes.

Potent and Broadly Cross-Reactive Anti-TBEV Antibodies

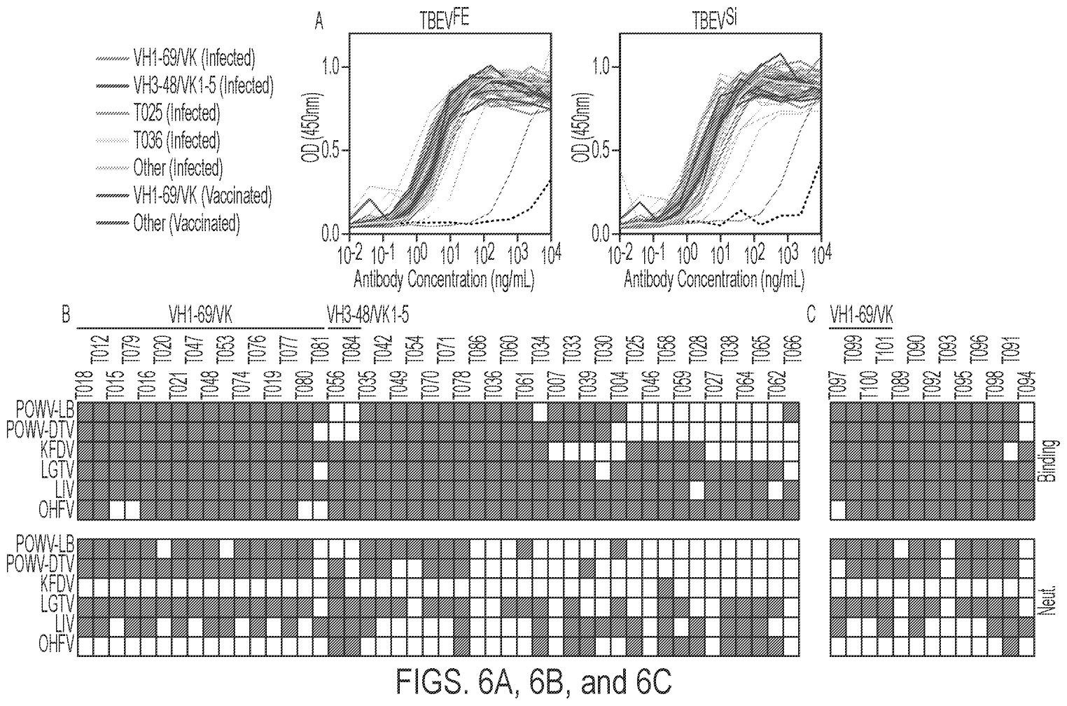

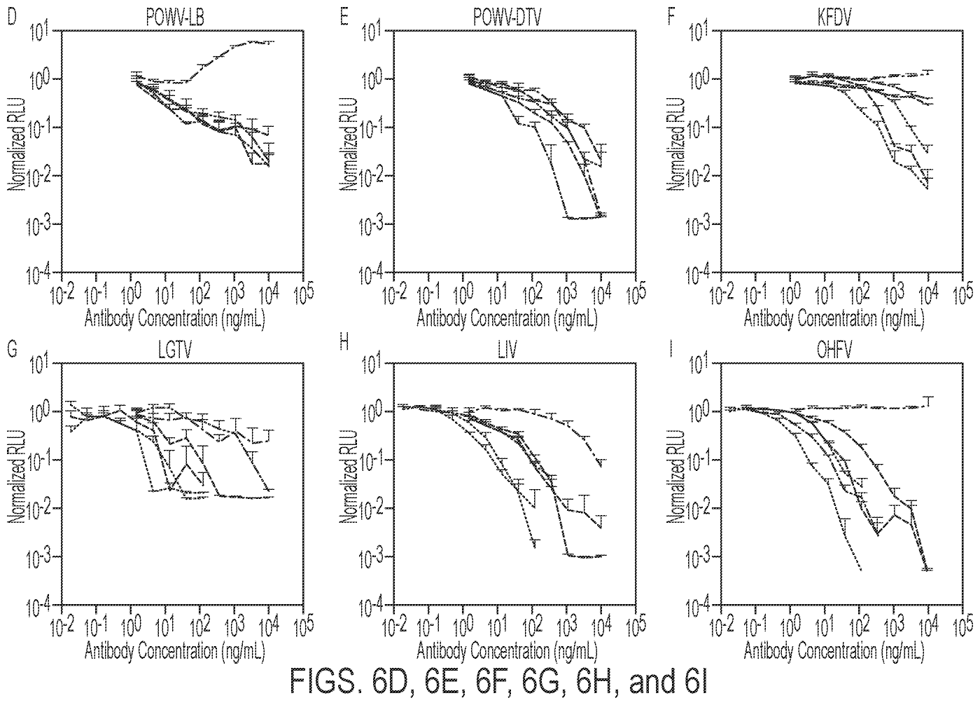

Fifty-nine antibodies (46 from convalescent and 13 from vaccinated donors, Table 4) were cloned, modified through recombinant DNA methods, expressed, and tested in ELISA for binding to EDIII proteins corresponding to all 3 TBEV subtypes: Western European (TBEV^{WE}), Far Eastern (TBEV^{FE}), and Siberian (TBEV^{Si}; FIGS. 5A and 6A, and Table 5). All but one of the 59 antibodies bound to all 3 EDIIIs with similar half-maximal effective concentrations (EC₅₀) ranging from 0.2 to 12 ng/mL (FIG. 5B, Table 5).

When tested for neutralizing activity against TBEV RVPs, 43 out of 46 antibodies obtained from infected donors neutralized, with IC₅₀s as low as 0.02 ng/mL (FIGS. 5C and 5D, Table 5). In contrast, the best antibody obtained from vaccinated donors had an IC₅₀ of 8.3 ng/mL. Seven antibodies, all isolated from infected donors, were potent neutralizers of TBEV with IC₅₀s below 1 ng/mL (FIG. 5D). Four of these antibodies were also evaluated for neutralization of authentic TBEV (FIGS. 5E and 5F). All four antibodies showed potent activity with IC₅₀s ranging from 35.9 to 268.8 ng/mL (Table 5).

To determine whether the TBEV antibodies cross-react with related viruses, the TBEV antibodies were screened them at a single concentration (1 μg/mL) for binding to the EDIIIs of Langat (LGTV), louping ill (LIV), Omsk hemorrhagic fever (OHFV), Kyasanur forest disease (KFDV), and Powassan lineage I and II viruses (POWV-DTV and POWV-LB; see Methods and FIGS. 6B and 6C). Broad cross-reactivity was observed for many of the antibodies tested (FIG. 6B). To determine whether the antibodies are also broadly neutralizing, the antibodies were screened against RVPs corresponding to the same panel of tick-borne viruses. When tested at a concentration of 1 μg/ml most of the IGHV1-69 antibodies neutralized LGTV, LIV, POWV-LB, and POWV-DTV and one of the IGVH3-48/IGVK1-5 antibodies neutralized all RVPs except POWV-LB (FIGS. 6B and 6C). IC₅₀s against the flavivirus RVP panel were in the single digit ng/mL range for several of the cross-reactive antibodies (FIG. 5G and FIGS. 6D-I; Table 5). For example, an IGVH3-48/IGVK1-5 antibody, T056, is a potent neutralizer of LGTV, LIV, and OHFV, with IC₅₀ values equal to or less than 1 ng/mL. It was concluded that some TBEV neutralizing antibodies are broadly active against tick-borne flaviviruses.

Antibody T036 Promotes TBEV Infection

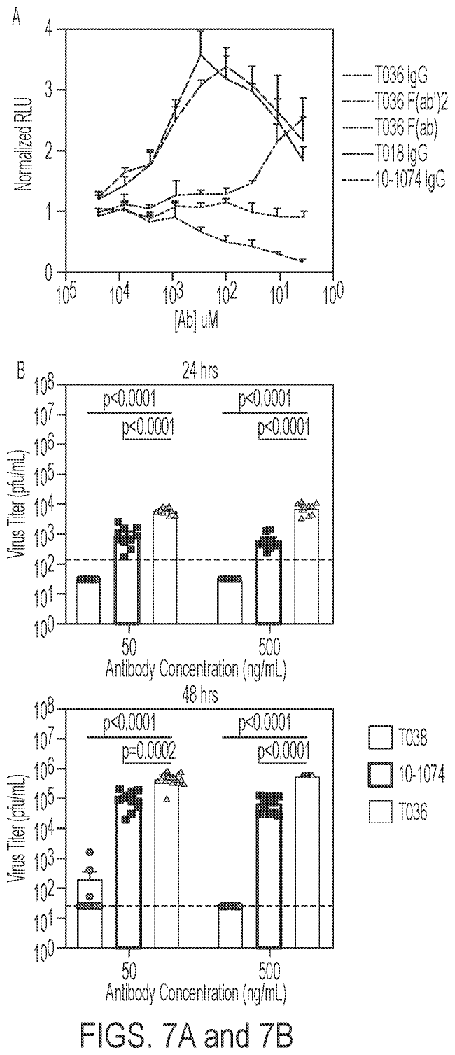

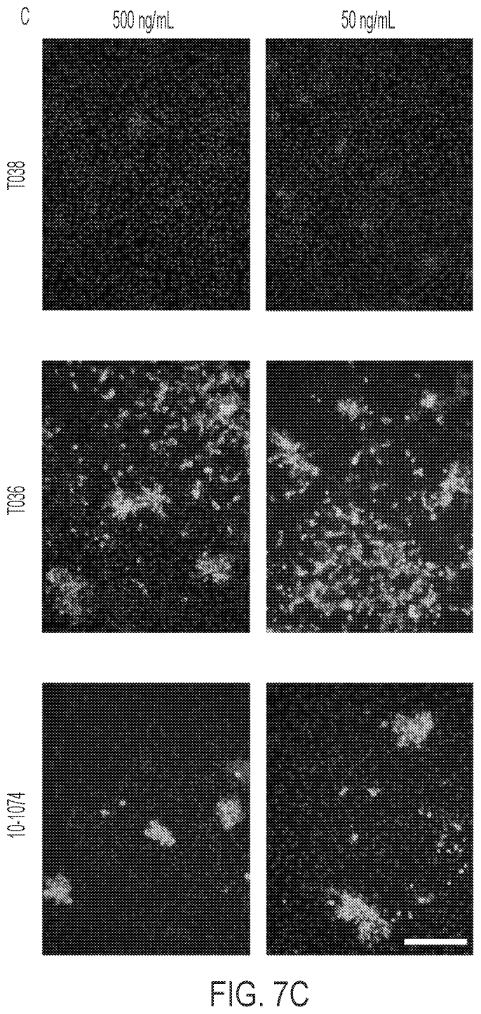

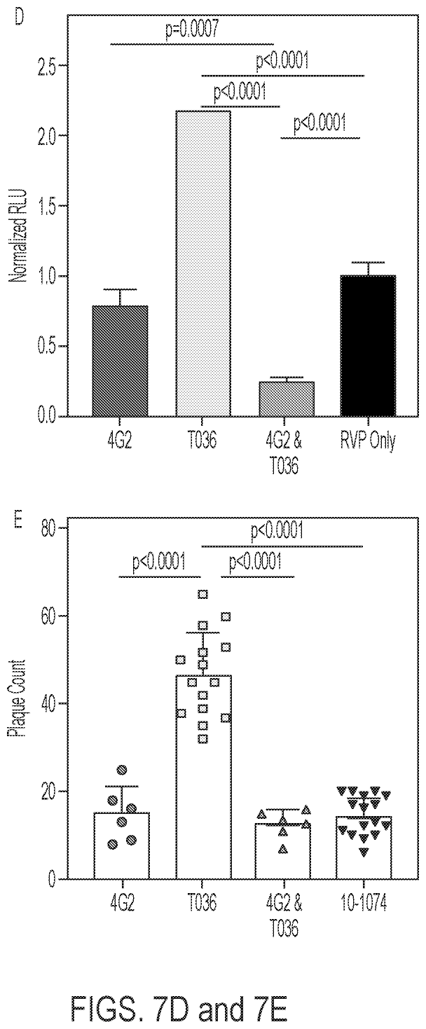

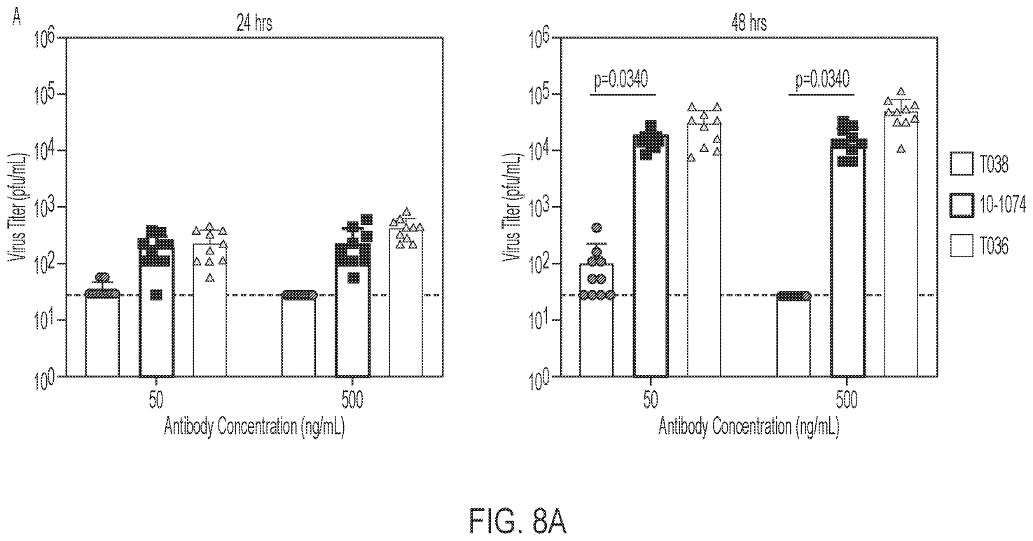

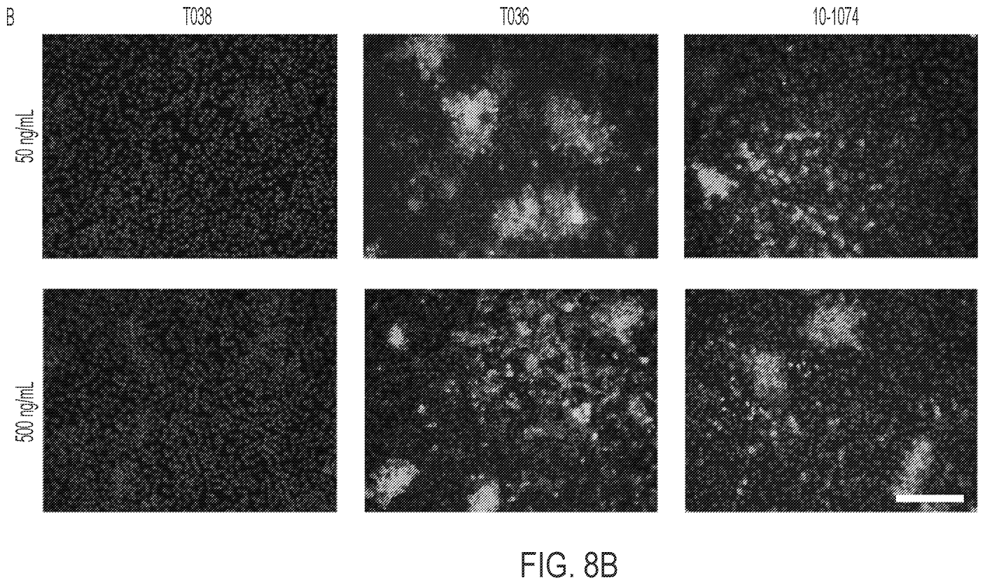

In contrast to the other antibodies, T036 displayed dose-dependent enhancement of TBEV and POWV-LB RVP infection (FIG. 7A and FIG. 6D). Enhancement was also observed with T036 F(ab′)2 and F(ab), indicating that neither bivalent binding nor the Fc domain is required for enhancement (FIG. 7A). To determine whether T036 enhances infection of authentic TBEV, plaque reduction assays were performed. Addition of T036 increased virus growth when compared to T038 (a neutralizing antibody) or isotype control (FIGS. 7B and 7C; FIGS. 8A and 8B).

A5, a mouse monoclonal antibody to envelope domain II (EDII), enhances viral fusion by exposing the fusion loop of the E protein. Its activity can be inhibited by 4G2, a fusion loop-directed mouse monoclonal (Haslwanter, D., et al., (2017) PLoS Pathog 13, e1006643-e1006643. To determine whether enhancement of infection by T036, a human anti-EDIII antibody, can also be inhibited by 4G2, TBEV RVP infection was measured in the presence of T036 or 4G2 alone or the combination (FIG. 7D). Consistent with a role for T036 in exposing a fusion loop epitope, enhancement was inhibited when both antibodies were present, but 4G2 alone had no detectable effect (FIG. 7D). Similar results were obtained using authentic TBEV in virus binding assays (FIG. 7E). The results indicate that T036 enhances TBEV infection by a mechanism that requires exposure of the fusion loop of the E protein.

Antibody T025 Structure Reveals Binding to a Lateral Ridge Epitope

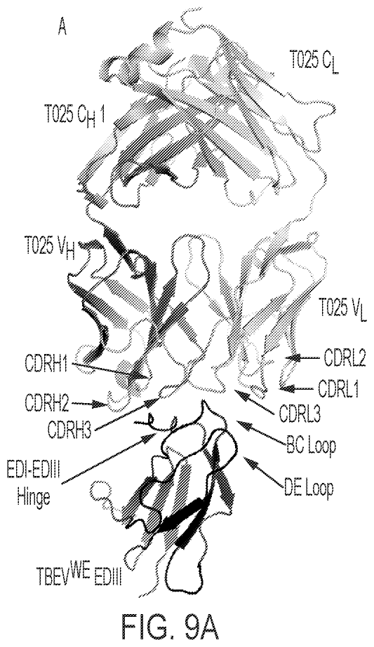

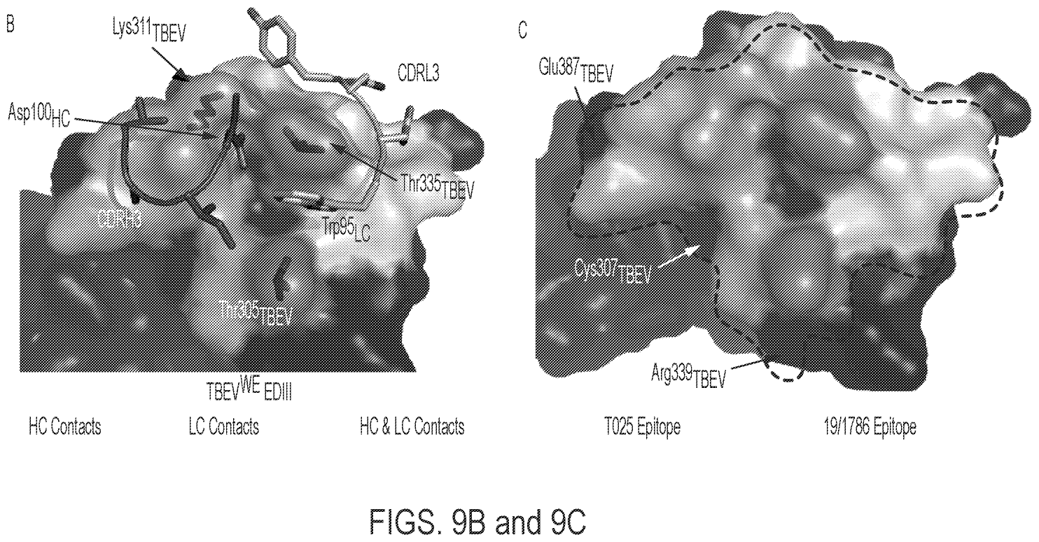

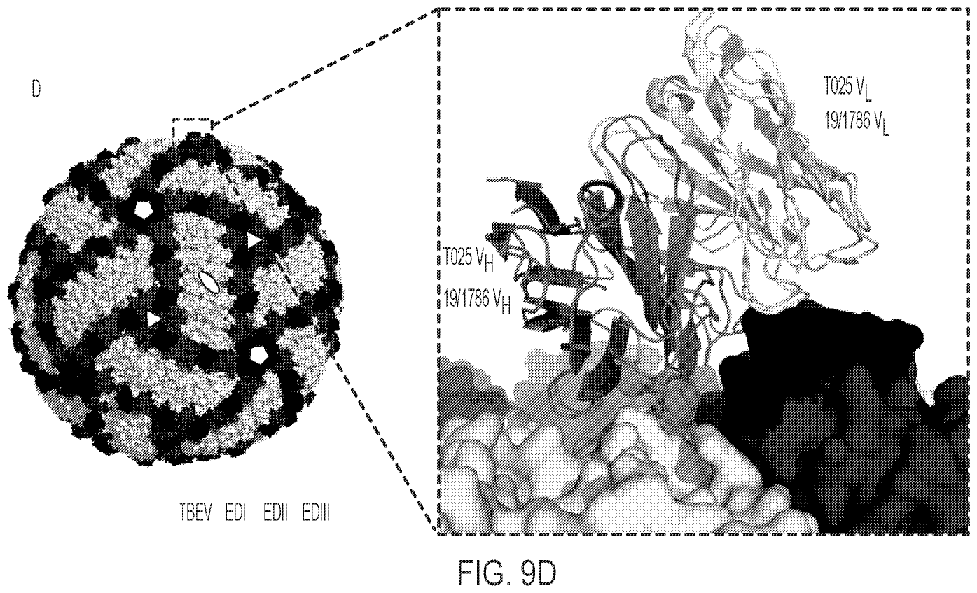

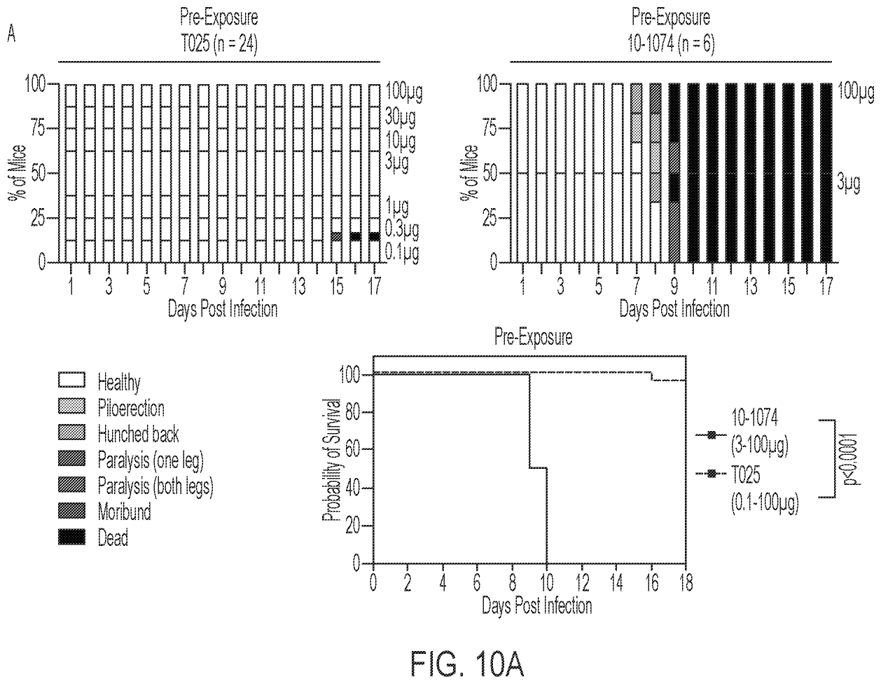

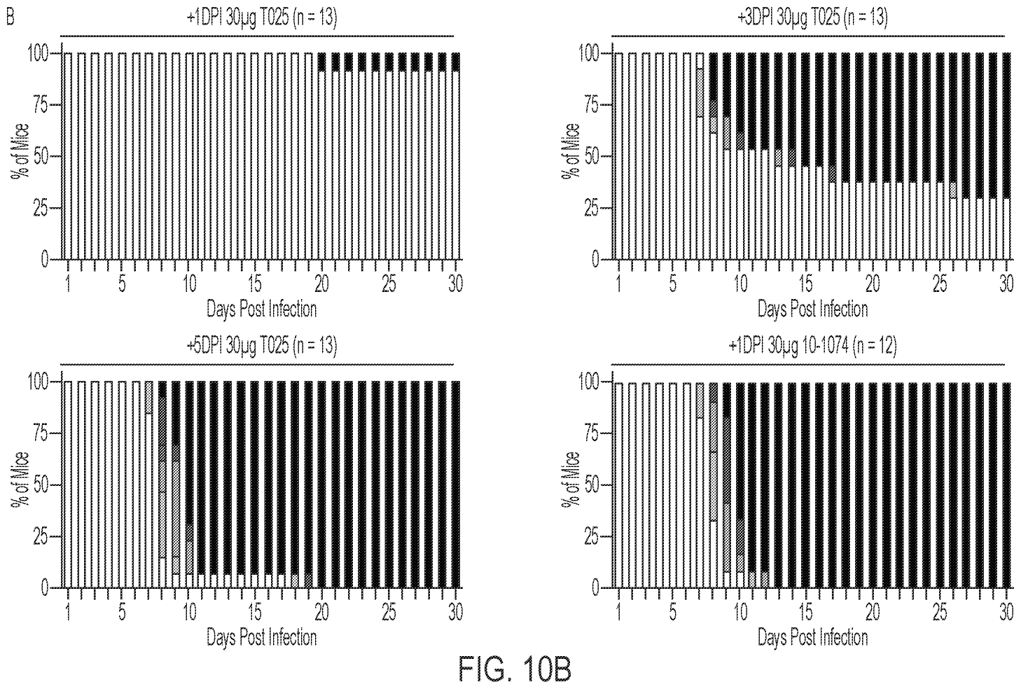

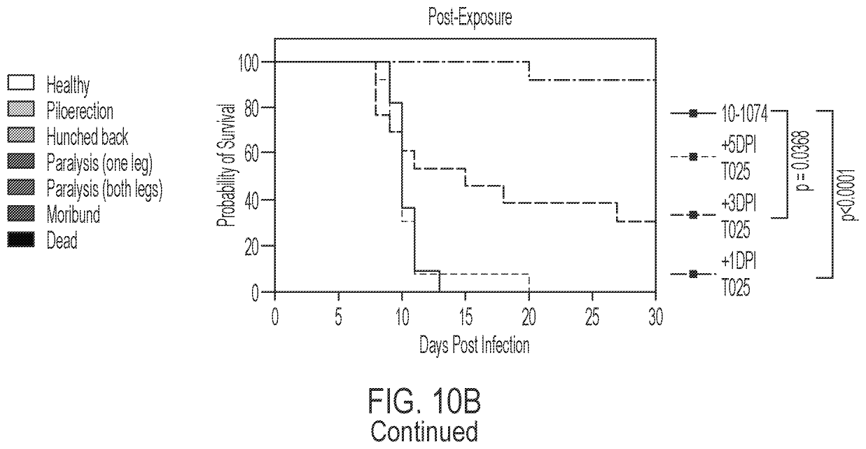

To gain insights into the mechanism of neutralization by human anti-TBEV antibodies, crystal structures of the Fab of T025, a broad and potent antibody, in complex with the EDIII domains of all three subtypes of TBEV were solved (FIGS. 9A-D and 10A-B). The structure of the T025 Fab-TBEV^{WE} EDIII complex revealed that the antibody binds near the lateral ridge of EDIII in the proximity of the EDI-EDIII hinge region, making both heavy and light chain contacts to the EDI-EDIII hinge and the BC loop, and light chain contacts to the DE loop of the EDIII (FIG. 9A). The antibody contacts EDIII using CDRH2, CDRH3, CDRL1, and CDRL3, and buries 598 Å² of surface area on the EDIII (333 Å² by the VH and 265 Å² by the V_{L}). T025 inserts Asp100_{HC} and Trp94_{LC} into a cleft in the EDIII, making a salt bridge (Asp100_{HC}-Lys311_{EDIII}) and hydrogen bonds to the EDIII (FIG. 9B). Crystal structures of T025 Fab in complex with TBEV^{FE} EDIII and TBEV^{Si} EDIII were similar to the T025-TBEV^{WE} EDIII structure (RMSDs=0.53 Å for 519 Cα atoms and 0.25 Å for 516 Cα atoms, respectively), consistent with 100% sequence conservation between these three strains of the virus in the epitope residues (FIG. 10A-B).

The T025 Fab-TBEV^{WE} structure was compared to a 3.9 Å cryo-EM structure of a mouse monoclonal antibody (19/1786) bound to the TBEV virion (Füzik, T., et al., (2018) Nat Commun 9, 436). T025 and 19/1786 are related by <65% amino acid sequence identity in the V_{H}V_{L} and 47% in the CDRs, but structural alignment of the structures by the Cα atoms of the EDIIIs shows that the two antibodies recognize similar epitopes (FIG. 9C) and adopt similar poses (FIG. 9D). The lower resolution cryo-EM structure can therefore be used to deduce details about how T025 binds to and neutralizes the virus. In addition to contacts with EDIII, 19/1786 interacted with either the EDI or EDII of a neighboring subunit (Füzik, T., et al., (2018) Nat Commun 9, 436). This, taken together with the relatively low buried surface area on the EDIII by T025 (˜600 Å² compared with a typical value of ˜1100 Å²) (Ramaraj, T., et al., (2012) Biochim Biophys Acta 1824, 520-532), indicates that T025 also contacts neighboring domains on a native virion. It is also likely that in common with recognition of virions by 19/1786, 120 of the 180 EDIIIs on the virion could be bound by T025.

Antibody T025 Prevents and Treats Infection in Mice

To determine whether anti-EDIII antibodies can protect against infection in vivo, prophylaxis experiments were performed in BALB/c mice. The mice received graded doses of T025 (100 to 0.1 μg per mouse) 24 hours before challenge with 10² pfu of TBEV (a lethal dose). All mice treated with the isotype control antibody died by day 10 (n=6). In contrast, even the lowest dose of T025 was protective; all but 1 of the 24 mice receiving the antibody survived (p<0.0001, FIG. 10A). To test the T025's potential for therapy, BALB/c mice were infected with 10² pfu of TBEV and then injected with 30 μg of T025 or isotype control 1, 3 or 5 days later (FIG. 10B). All 12 control mice succumbed to the infection by day 13. In contrast, 12 out of 13 mice treated with T025 on day 1, and 4 out of 13 mice treated on day 3 after infection survived. All mice treated with T025 5 days after infection failed to respond. Thus T025, a broadly neutralizing human anti-TBEV antibody, is efficacious in prevention and treatment of TBEV infection in BALB/c mice.

### DISCUSSION

Tick-borne flaviviruses can cause fulminant encephalitis for which there is no effective therapy. This group of viruses is a growing public health concern in Europe, Asia, and North America. Among disease-causing tick-borne flaviviruses, TBEV is prevalent in Central Europe and Russia. Although there is a great deal of information on the polyclonal humoral immune response to TBEV (Albinsson, B., et al., (2018) Euro Surveill 23, pii=17-00838; Holzmann, H., (2003) Vaccine 21 S36-S40; Matveeva, V. A., et al., (1995) Immunol 46, 1-4; McAuley, A. J., et al., (2017) NPJ Vaccines 2, 5; Remoli, M. E., et al., (2014) Pathog Dis 73, 1-3), there is little or no understanding of the molecular nature of the neutralizing antibody response induced by natural infection or vaccination. This example describes 776 antibodies obtained from the memory B cells of 6 recovered and 3 vaccinated individuals, among which are several broad and potent neutralizers of tick-borne flaviviruses. The data provide insights into the human antibody response to TBEV and related pathogens, as well as mechanisms of antibody-induced neutralization and enhancement. Finally, broad and potent neutralizing human monoclonal antibodies are highly effective for protection and therapy in vivo and have significant potential for clinical use.

Human neutralizing antibody responses to pathogens frequently converge on the same IGV genes. Examples include neutralizing antibodies to HIV-1, influenza, Zika, hepatitis B, and SARS-CoV-2 viruses (Robbiani, D. F., et al., (2017) Cell 169, 597-609 e511; Robbiani, D. F., et al., (2020) Nature 584, 437-442; Scheid, J. F., et al., (2011) Science (New York, NY) 333, 1633-1637; Tiller, T., et al., (2007) Immunity 26, 205-213; Wang, Q., et al., (2020) Cell Host Microbe 28 335-349.e336; West, A. P., et al., (2012) Proc Natl Acad Sci USA 109, E2083-2090). Antibodies to the EDIII of TBEV produced by different individuals show strong homology that, like SARS-CoV-2 antibodies, extends beyond IGV heavy and light chain gene pairing and includes the CDR3 regions.

Among the neutralizing antibodies to TBEV, VH1-69 and VH3-48 were highly over-represented. VH1-69 was paired with a variety of different light chain genes to produce neutralizing antibodies that were found among vaccinees and recovered individuals. This group of antibodies varied broadly in neutralizing activity ranging from IC₅₀ 12-1180 ng/mL (geometric mean 186.2 ng/mL). VH1-69 antibodies are highly represented in the human repertoire and are also common among broadly neutralizing antibodies to influenza, hepatitis C and HIV-1 (Chen, F., et al., (2019) Curr Opin Virol 34, 149-159). Anti-TBEV VH3-48 antibodies differed from VH1-69 in that they were always paired with the same light chain, VK1-5. VH3-48 antibodies were also more potent than VH1-69 with IC₅₀s ranging from 0.5-7.3 ng/mL (geometric mean 2 ng/mL), and they were only found in convalescent individuals. The absence of this class of potent antibodies in the vaccinees examined is consistent with the lower levels of serum neutralizing potency in this group. Finally, VH3-48 antibodies are also potent neutralizers of several related tick-borne flaviviruses, including KFDV, LGTV, LIV, and OHFV, with IC₅₀s 1-36 ng/mL.

Antibodies to a number of different flaviviruses, including dengue and Zika, can be protective if administered before and even after infection (Robbiani, D. F., et al., (2017) Cell 169 597-609 e511; Xu, M., et al., (2017) NPJ Vaccines 2, 2). In Russia and Kazakhstan, administration of TBEV hyperimmune plasma is recommended for post-exposure prophylaxis for individuals that present within 3 days of a tick bite (2008; Pen'evskaia, N. A., and Rudakov, N. V., (2010) Med Parazitol (Mosk) 53-59). The efficacy of this intervention may vary from batch to batch of donor plasma (Rabel, P. O., et al., (2012) Clin Vaccine Immunol 19 623-625; Ruzek, D., et al., (2019) Antiviral Res 164, 23-51), and its use was discontinued in some countries after a small number of adverse events and concerns about the possibility of antibody-dependent enhancement of disease (Arras, C., et al., (1996) Lancet 347, 1331; Kluger, G., et al., (1995) Lancet 346, 1502; Waldvogel, K., et al., (1996) Eur J Pediatr 155, 775-779). Mouse monoclonal antibodies can also protect against TBEV but have not been tested in the clinic (Baykov, I. K., et al., (2014) Vaccine 32, 3589-3594; Levanov, L. N., et al., (2010) Vaccine 28, 5265-5271; Matveev, A., et al., (2020) Vaccine 38, 4309-4315). The experiments extend previous work by uncovering human monoclonal antibodies that prevent infection in mice even when administered at doses as low as ˜0.005 mg/Kg. Notably, these antibodies also suppress disease in mice even when administered 3 days after infection at a dose of ˜1.5 mg/Kg.

Sub-optimal antibody levels to other flaviviruses, most importantly dengue and possibly Zika, are associated with antibody-dependent enhancement (ADE) of disease (Dejnirattisai, W., et al., (2016) Nat Immunol 17, 1102-1108; Harrison, S. C., (2016) Nat Immunol 17, 1010-1012; Katzelnick, L. C., et al., (2017) Science (New York, NY) 358, 929-932; Katzelnick, L. C., et al., (2020) Science (New York, NY) 369, 1123-1128; Sabin, A. B., (1950) Bacteriol Rev 14, 225-232; Stettler, K., et al., (2016) Science (New York, NY) 353, 823-826). ADE has also been discussed as an explanation for the fulminant encephalitis that occurs in a fraction of individuals after TBEV infection, including vaccine break-throughs (Ruzek, D., et al., (2019) Antiviral Res 164, 23-51). ADE in dengue infection is thought to be mediated by immune-complexes that enhance pathogen entry into Fc receptor-expressing cells (Halstead, S. B., (2014) Microbiol Spectr 2). However, antibodies can also enhance infection by inducing conformational changes in the viral surface proteins that facilitate engagement of the viral fusion machinery (Guillon, C., et al., (2002) J Virol 76, 2827-2834; Wan, Y., et al., (2020) J Virol 94, e02015-02019; Winarski, K. L., et al., (2019) Proc Natl Acad Sci USA 116, 15194-15199). Indeed, a mouse monoclonal antibody to TBEV has been identified, which enhances by this mechanism (Haslwanter, D., et al., (2017) PLoS Pathog 13, e1006643-e1006643). The discovery that humans infected with TBEV produce antibodies that promote viral infection in vitro raises the question of whether such antibodies may play a role in TBE pathogenesis or the rare adverse events seen after plasma administration in the clinic.

Current TBEV vaccines were developed over 30 years ago and consist of inactivated virus grown on chick embryo cells. Vaccination is TBEV-specific, requires priming and two boosts, and results in 90-100% seroconversion depending on the vaccine used (Loew-Baselli, A., et al., (2009) Hum Vacc 5, 551-556; Maikova, G. B., et al., (2019) J Med Virol 91, 190-200; Vorovitch, M. F., et al., (2019) Adv Virol 2019, 5323428). Additional boosts are recommended every 3-5 years for the lifetime of the individual. The existence of broad and potent VH3-48 antibodies indicates that next-generation vaccines specifically designed to target the epitope recognized by these antibodies might be universally effective against TBEV, KFDV, LGTV, LIV, and OHFV. Finally, potent human antibodies with broad activity against tick-borne flaviviruses have significant potential for clinical use in individuals that are at high risk and do not respond to the vaccine and for therapy in the early stages of infection.

TABLE 1A

| | Year of | Age at | | Vaccination | | Hospital- | | | IgG | |
|---|---|---|---|---|---|---|---|---|---|---|
| ID | Diagnosis | Diagnosis | Sex | History | Tick Bite | ization (days) | Severity | IgM (IP) | (VIEU/ml) | Notes |
| 1 | 2011 | 56 | female | not known | not known | | mild | 4.4 | 687.15 | |
| 2 | 2011 | 12 | male | not known | not known | | mild | 3.7 | 634.94 | |
| 3 | 2011 | | | | | | | | | |
| 4 | 2011 | 26 | male | not known | not known | | moderate | 3.8 | 905.79 | |
| 5 | 2011 | | | | | | | | | |
| 6 | 2011 | 41 | female | not known | not known | 3 | mild | 4 | 905.79 | |
| 7 | 2011 | 75 | male | not known | yes | 7 | severe | 3.6 | 934.2225 | |
| 8 | 2011 | 65 | male | not known | not known | 10 | severe | 3.5 | 252.54 | diabetes |
| 9 | 2011 | 65 | male | no | yes | | severe | 3.8 | 905.79 | |
| 10 | 2011 | 64 | male | no | yes | 7 | severe | 3.7 | 905.79 | |
| 11 | 2011 | 77 | male | no | yes | 27 | severe | 3.8 | 252.54 | death due to |
| | | | | | | | | | | pulmonary |
| | | | | | | | | | | embolism |
| 12 | 2011 | 10 | male | no | yes | 7 | mild | 2.7 | 934.2225 | |
| 13 | 2011 | 60 | female | no | yes | 16 | severe | 4.3 | 905.79 | |
| 15 | 2011 | | | | | | | | | |
| 16 | 2011 | 66 | female | no | yes | 10 | severe | 2.8 | 388.59 | |
| 17 | 2011 | 42 | female | not known | not known | 24 | severe | 2.3 | 230.7225 | paresis; |
| | | | | | | | | | | longterm |
| | | | | | | | | | | convalescence |
| 18 | 2011 | | | | | | | | | no data |
| | | | | | | | | | | available |
| 19 | 2011 | 54 | male | no | yes | 10 | severe | 5.6 | 563.4924 | |
| 20 | 2011 | | | | | | | | | |
| 21 | 2011 | 49 | female | not known | yes | 8 | moderate | 3.5 | 962.9 | |
| 22 | 2011 | | | | | | | | | |
| 23 | 2011 | 74 | male | not known | yes | 22 | severe | 3 | 740.34 | |
| 24 | 2011 | 66 | male | no | yes | 11 | severe | 3.7 | 342.26 | |
| 25 | 2011 | 34 | male | not known | yes | 8 | moderate | 3.4 | 435.9 | |
| 26 | 2011 | 62 | male | no | yes | 19 | severe | 2.6 | 905.79 | |
| 27 | 2011 | 35 | female | not known | yes | 13 | severe | 3.2 | 252.54 | |
| 28 | 2011 | 44 | male | no | yes | 10 | moderate | 3.3 | 905.79 | |
| 29 | 2011 | 24 | female | no | yes | 12 | severe | 3.7 | 905.79 | recent |
| | | | | | | | | | | travel to |
| | | | | | | | | | | Croatia, |
| | | | | | | | | | | herpes |
| | | | | | | | | | | simplex |
| | | | | | | | | | | reactivation |
| | | | | | | | | | | during TBE |
| 30 | 2011 | 45 | female | no | yes | 13 | severe | 3.4 | 252.54 | |
| 31 | 2011 | 35 | female | not known | yes | 9 | severe | 3.5 | 905.79 | light paresis |
| 32 | 2011 | | | | | | | | | no data |
| | | | | | | | | | | available |
| 33 | 2011 | 69 | male | no | yes | 9 | moderate | 4 | 934.2225 | recent |
| | | | | | | | | | | travel to |
| | | | | | | | | | | Sicily, |
| | | | | | | | | | | metabolic |
| | | | | | | | | | | disorder |
| 34 | 2011 | | | | | | | | | no data |
| | | | | | | | | | | available |
| 35 | 2011 | 24 | female | no | yes | 9 | severe | 3.5 | 905.79 | leptospira |
| | | | | | | | | | | coinfection |
| 36 | 2011 | 44 | male | no | yes | 8 | severe | 3.7 | 905.79 | |
| 37 | 2011 | 32 | female | not known | yes | 8 | mild | 4 | 962.9 | |
| 38 | 2011 | 40 | female | not known | yes | 19 | severe | 3.9 | 962.9 | paresis |
| 39 | 2011 | 35 | female | no | yes | 9 | mild | 3.7 | 962.9 | |
| 40 | 2011 | 56 | female | no | yes | 10 | severe | 2.7 | 209.15 | |
| 41 | 2011 | 15 | male | not known | not known | 0 | mild | 3.7 | 962.9 | discharged |
| | | | | | | | | | | AMA |
| 42 | 2011 | 36 | female | no | yes | 11 | moderate | 3 | 962.9 | |
| 43 | 2011 | | | | | | | | | |
| 44 | 2011 | 67 | female | no | yes | 10 | severe | 3.5 | 905.79 | paresis |
| 45 | 2011 | | | | | | | | | |
| 46 | 2011 | 35 | female | no | yes | 7 | mild | 4.2 | 849.66 | |
| 47 | 2011 | | | | | | | | | |
| 48 | 2011 | | | | | | | | | |
| 49 | 2011 | 42 | female | no | yes | 12 | severe | 3.8 | 905.79 | recent |
| | | | | | | | | | | travel to |
| | | | | | | | | | | Croatia |
| 50 | 2011 | | | | | | | | | |
| 51 | 2011 | 89 | female | not known | not known | 16 | severe | 4 | 849.66 | diabetes |
| | | | | | | | | | | mellitus |
| 52 | 2011 | 73 | male | no | not known | 14 | severe | 3.8 | 634.94 | |
| 53 | 2011 | 26 | male | no | no | 8 | mild | 4.3 | 533.46 | |
| 54 | 2011 | | | | | | | | | |
| 55 | 2011 | 39 | female | no | yes | 10 | moderate | 3.6 | 905.79 | |
| 56 | 2011 | | | | | | | | | |
| 57 | 2011 | 29 | female | no | no | 10 | mild | 3 | 905.79 | psoriasis |
| 58 | 2011 | 55 | female | no | no | 4 | mild | 3.6 | 962.9 | |
| 59 | 2011 | 30 | female | no | yes | 9 | severe | 3.1 | 905.79 | |
| 60 | 2011 | 62 | male | no | yes | 8 | moderate | 3.6 | 905.79 | |
| 61 | 2011 | 49 | male | no | yes | 8 | mild | | | |
| 62 | 2018 | 38 | male | no | yes | 9 | mild | | | |
| 63 | 2018 | 43 | male | no | yes | 7 | mild | | | |
| 64 | 2018 | 55 | male | no | not known | 17 | mild | | | |
| 65 | 2018 | 55 | male | no | yes | 10 | mild | | | |
| 66 | 2018 | 67 | female | no | not known | 13 | severe | | | |
| 67 | 2018 | 54 | male | no | yes | 234 | severe | | | paresis |
| 68 | 2018 | 46 | male | no | not known | 9 | mild | | | |
| 69 | 2018 | 31 | male | no | yes | 9 | mild | | | |
| 70 | 2018 | 45 | female | no | yes | 7 | mild | | | |
| 71 | 2018 | 35 | female | no | yes | | mild | | | |
| 73 | 2018 | 52 | male | no | yes | 15 | severe | | | paresis |
| 74 | 2018 | 11 | male | no | yes | 15 | mild | | | |
| 75 | 2018 | 10 | male | no | yes | 11 | mild | | | |
| 76 | 2018 | 66 | male | no | yes | 7 | moderate | 4.7 | 962.9 | |
| 77 | 2018 | 60 | female | no | yes | 8 | moderate | 5.3 | 962.9 | unknown |
| | | | | | | | | | | neuroinfection |
| | | | | | | | | | | in 1966 |
| 78 | 2018 | 78 | male | no | yes | 29 | severe | 6.2 | 697.7096 | |
| 79 | 2018 | 32 | female | no | no | 9 | moderate | 4.8 | 209.15 | |
| 80 | 2018 | 39 | male | no | yes | 7 | severe | 5.5 | 655.7064 | |
| 81 | 2018 | 68 | male | not known | yes | 13 | severe | 4.5 | 296.91 | |
| 82 | 2018 | 71 | male | not known | yes | 16 | severe | 5.6 | 563.4924 | |
| 83 | 2018 | 45 | male | not known | not known | 9 | moderate | 2.9 | 162.5529 | |
| 84 | 2018 | 32 | male | no | yes | 6 | moderate | 5.1 | 629.7729 | |
| 85 | 2018 | 59 | female | no | yes | 8 | moderate | 3.8 | 158.3756 | |
| 86 | 2018 | 24 | male | no | yes | 10 | moderate | 5.2 | 426.3596 | |
| 87 | 2018 | 50 | male | no | yes | 8 | mild | 5.7 | 609.2025 | probable |
| | | | | | | | | | | ingestion of |
| | | | | | | | | | | unpasteurised |
| | | | | | | | | | | milk |
| | | | | | | | | | | products |
| 88 | 2018 | 34 | male | no | no | 8 | moderate | 5.9 | 708.3084 | |
| 89 | 2018 | 5 | male | no | yes | 6 | mild | 6.4 | 459.9225 | |
| 90 | 2017 | 31 | male | not known | not known | 1 | | 3.5 | 479.3169 | serum taken |
| | | | | | | | | | | in 2018 |
| | | | | | | | | | | during |
| | | | | | | | | | | reoccurrence |
| | | | | | | | | | | of |
| | | | | | | | | | | neuroinfection |
| | | | | | | | | | | symptoms |
| 91 | 2018 | 46 | female | | | | | 2.2 | 911.4569 | some data not |
| | | | | | | | | | | available, |
| | | | | | | | | | | hospitalized |
| | | | | | | | | | | in different |
| | | | | | | | | | | hospital |
| 92 | 2018 | 54 | female | no | not known | 6 | mild | 4.4 | 588.7889 | travel to |
| | | | | | | | | | | Croatia 2 |
| | | | | | | | | | | months |
| | | | | | | | | | | previously |
| 93 | 2018 | 66 | male | no | yes | | severe | 3.8 | 22.1784 | death due to |
| | | | | | | | | | | TBE |
| 94 | 2018 | 84 | male | no | yes | | severe | 5.6 | 25.637649 | death due to |
| | | | | | | | | | | TBE |
| 95 | 2018 | 71 | female | no | yes | 14 | moderate | 5.9 | 513.6344 | |
| 96 | 2018 | 65 | male | no | yes | 13 | severe | 5.7 | 609.2025 | |
| 97 | 2018 | 47 | male | no | yes | 11 | moderate | 6.3 | 274.6025 | |
| 98 | 2018 | 73 | male | no | yes | 11 | severe | 5.4 | 660.9225 | |
| 99 | 2018 | 40 | male | no | no | 11 | severe | 7.2 | 256.9329 | immunodeficient, |
| | | | | | | | | | | probable |
| | | | | | | | | | | ingestion of |
| | | | | | | | | | | unpasteurised |
| | | | | | | | | | | milk products |
| | | | | | | | | | | (goat) |
| 100 | 2018 | 44 | male | no | yes | 7 | moderate | 6.1 | 756.4881 | |
| 101 | 2018 | 35 | male | no | not known | 9 | severe | | 342.26 | |
| 102 | 2018 | 66 | female | no | yes | 17 | moderate | 5.9 | 573.5816 | atrial |
| | | | | | | | | | | fibrillation |
| | | | | | | | | | | during |
| | | | | | | | | | | hospitalization |
| | | | | | | | | | | for TBE |
| 104 | 2018 | 18 | male | yes | yes | 8 | severe | 6.6 | 962.9 | |
| 105 | 2018 | 32 | female | not known | not known | 0 | mild | 5.9 | 650.5001 | discharged |
| | | | | | | | | | | AMA |
| 106 | 2018 | 47 | male | no | no | 8 | moderate | 5.7 | 158.3756 | |
| 107 | 2018 | 6 | male | no | yes | 9 | mild | 5.9 | 614.3304 | |
| 108 | 2018 | 47 | male | no | yes | 5 | severe | 4.8 | 697.7096 | discharged |
| | | | | | | | | | | AMA |
| 109 | 2018 | 53 | female | no | yes | 10 | moderate | 4.8 | 484.19 | |
| 110 | 2018 | 70 | male | no | yes | 0 | mild | 5.3 | 402.6801 | |
| 111 | 2018 | 39 | male | no | yes | 8 | severe | 5.6 | 604.0844 | |
| 112 | 2018 | 63 | female | not known | not known | 9 | moderate | 5.7 | 729.6236 | |
| 113 | 2018 | 46 | male | not known | yes | 6 | mild | 6.9 | 538.4409 | |
| 114 | 2018 | 41 | male | not known | yes | 1 | mild | 5.4 | 634.94 | discharged |
| | | | | | | | | | | AMA |
| 115 | 2018 | 5 | male | no | yes | 6 | mild | 7.1 | 849.66 | |
| 116 | 2018 | 74 | male | no | yes | 8 | moderate | 5.1 | 573.5816 | |
| 117 | 2018 | 52 | male | no | yes | 17 | severe | 6.5 | 431.1249 | |
| 118 | 2018 | 9 | male | no | yes | 6 | mild | 5.9 | 939.9384 | |
| 119 | 2018 | 63 | female | not known | not known | 12 | severe | 3.1 | 388.59 | |
| 120 | 2018 | 51 | female | no | no | 11 | severe | 4.8 | 166.74 | |
| 121 | 2018 | 73 | female | no | yes | 16 | severe | 2.5 | 248.1569 | |
| 123 | 2018 | 45 | male | not known | no | 9 | severe | 6.8 | 187.8225 | |
| 124 | 2018 | 54 | male | no | yes | 8 | severe | 6.7 | 369.9404 | |
| 125 | 2018 | 42 | male | no | no | 7 | moderate | 6.9 | 310.4121 | |
| 126 | 2018 | 58 | male | no | yes | 12 | severe | 6.4 | 235.0664 | |
| 127 | 2018 | 71 | male | no | yes | 9 | moderate | 5.7 | 416.8584 | |
| 128 | 2018 | 50 | male | yes | no | 7 | moderate | 6 | 265.7481 | |
| 129 | 2018 | 76 | male | not known | not known | 11 | severe | 4.5 | 226.3884 | |
| 130 | 2018 | 72 | female | no | yes | 11 | moderate | 5.8 | 455.0984 | |
| 131 | 2018 | 78 | female | no | yes | 14 | severe | 5.5 | 346.8489 | |
| 132 | 2018 | 32 | male | no | yes | 7 | moderate | 5.9 | 469.6001 | |
| 133 | 2018 | 28 | male | no | no | 6 | severe | 6.1 | 416.8584 | |
| 134 | 2018 | 85 | female | not known | yes | 9 | severe | 4.1 | 121.2209 | |
| 135 | 2018 | 68 | female | not known | not known | 9 | severe | 5.6 | 548.4321 | |
| 136 | 2018 | 58 | female | no | yes | 9 | severe | 6 | 479.3169 | |
| 137 | 2018 | 47 | female | no | not known | 8 | severe | 3.4 | 383.9129 | |
| 138 | 2018 | 58 | female | no | yes | 8 | severe | 3.9 | 196.3241 | |
| 139 | 2018 | 49 | male | no | no | 7 | moderate | 5.4 | 265.7481 | |
| 140 | 2018 | 51 | male | no | yes | 11 | severe | 4.2 | 204.8649 | |
| 141 | 2018 | 84 | female | no | no | 12 | severe | 6.1 | 256.9329 | |
| 142 | 2018 | 64 | male | no | yes | 8 | moderate | 3.9 | 435.9 | |
| 143 | 2018 | 58 | male | no | no | 15 | severe | 4.7 | 292.4289 | infected |
| | | | | | | | | | | travelling in |
| | | | | | | | | | | Czech |
| | | | | | | | | | | Republic |
| 144 | 2018 | 65 | male | yes | yes | 4 | mild | 2.4 | 337.6809 | |
| 145 | 2018 | 66 | female | no | yes | 9 | severe | 4.5 | 479.3169 | |

Cells left blank where information is unknown or unavailable.
AMA: against medical advice
IgM: measured using EIA TBE Virus IgM kit (TestLine Clinical Diagnostics, TBM096); negative < 0.9 (IP); borderline result 0.9 to 1.1; 1.1 < positive
IgG: manufacturers recommendation; measured using EIA TBE Virus IgG kit (TestLine Clinical Diagnostics, TBG096); negative < 76.89 VIE/ml; borderline result 76.89 to 92.87; 92.87 < positive

TABLE 1B

| | Year of | | | Number of TBEV | |
|---|---|---|---|---|---|
| | Sample | | | Vaccination | Date of |
| ID | Collection | Age | Sex | Doses | Last Dose |
| V3 | 2018 | 31 | female | not known | not known |
| V4 | 2018 | 64 | female | 3 | 2006 |
| V5 | 2018 | 34 | female | 3 | 2003 |
| V6 | 2018 | 37 | female | not known | 1991 |
| V7 | 2018 | 22 | female | 3 | 2016 |
| V8 | 2018 | 21 | female | not known | 2013 |
| V9 | 2018 | 28 | female | not known | not known |
| V11 | 2019 | 24 | female | 2 | 2017 |
| V12 | 2019 | 29 | female | 7 | 2018 |
| V16 | 2019 | 24 | male | 4 | 2018 |

Lengthy table referenced here
US20240002480A1-20240104-T00001
Please refer to the end of the specification for access instructions.

Lengthy table referenced here
US20240002480A1-20240104-T00002
Please refer to the end of the specification for access instructions.

Lengthy table referenced here
US20240002480A1-20240104-T00003
Please refer to the end of the specification for access instructions.

Lengthy table referenced here
US20240002480A1-20240104-T00004
Please refer to the end of the specification for access instructions.

Lengthy table referenced here
US20240002480A1-20240104-T00005
Please refer to the end of the specification for access instructions.

Lengthy table referenced here
US20240002480A1-20240104-T00006
Please refer to the end of the specification for access instructions.

Lengthy table referenced here
US20240002480A1-20240104-T00007
Please refer to the end of the specification for access instructions.

Lengthy table referenced here
US20240002480A1-20240104-T00008
Please refer to the end of the specification for access instructions.

Lengthy table referenced here
US20240002480A1-20240104-T00009
Please refer to the end of the specification for access instructions.

Lengthy table referenced here
US20240002480A1-20240104-T00010
Please refer to the end of the specification for access instructions.

Lengthy table referenced here
US20240002480A1-20240104-T00011
Please refer to the end of the specification for access instructions.

Lengthy table referenced here
US20240002480A1-20240104-T00012
Please refer to the end of the specification for access instructions.

Lengthy table referenced here
US20240002480A1-20240104-T00013
Please refer to the end of the specification for access instructions.

LENGTHY TABLES
The patent application contains a lengthy table section. A copy of the table is available in electronic form from the USPTO web site (https://seqdata.uspto.gov/?pageRequest=docDetail&DocID=US20240002480A1). An electronic copy of the table will also be available from the USPTO upon request and payment of the fee set forth in 37 CFR 1.19(b)(3).

What is claimed is:

1. An isolated anti-tick-borne encephalitis virus (TBEV) antibody or antigen-binding fragment thereof that binds specifically to a TBEV antigen.

2. The antibody or antigen-binding fragment thereof of claim 1, wherein the TBEV antigen comprises a lateral ridge of domain III of the E protein (EDIII).

3. The antibody or antigen-binding fragment thereof of any one of the preceding claims, wherein the antibody or antigen-binding fragment thereof is capable of neutralizing a plurality of TBEV strains.

4. The antibody or antigen-binding fragment thereof of any one of the preceding claims, comprising three heavy chain complementarity determining regions (HCDRs) (HCDR1, HCDR2, and HCDR3) of a heavy chain variable region having an amino acid sequence of SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, or 117; and three light chain CDRs (LCDR1, LCDR2, and LCDR3) of a light chain variable region having the amino acid sequence of SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, or 118.

5. The antibody or antigen-binding fragment thereof of any one of the preceding claims, comprising:
a heavy chain variable region having an amino acid sequence with at least 75% identity to the amino acid sequence of SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, or 117; or having the amino acid sequence of SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, or 117; and
a light chain variable region having an amino acid sequence with at least 75% identity to the amino acid sequence of SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, or 118; or having the amino acid sequence of SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, or 118.

6. The antibody or antigen-binding fragment thereof of any one of the preceding claims, comprising a heavy chain variable region and a light chain variable region that comprise the respective amino acid sequences of SEQ ID NOs: 1-2, 3-4, 5-6, 7-8, 9-10, 11-12, 13-14, 15-16, 17-18, 19-20, 21-22, 23-24, 25-26, 27-28, 29-30, 31-32, 33-34, 35-36, 37-38, 39-40, 41-42, 43-44, 45-46, 47-48, 49-50, 51-52, 53-54, 55-56, 57-58, 59-60, 61-62, 63-64, 65-66, 67-68, 69-70, 71-72, 73-74, 75-76, 77-78, 79-80, 81-82, 83-84, 85-86, 87-88, 89-90, 91-92, 93-94, 95-96, 97-98, 99-100, 101-102, 103-104, 105-106, 107-108, 109-110, 111-112, 113-114, 115-116, or 117-118.

7. The antibody or antigen-binding fragment thereof of any one of the preceding claims, wherein the antibody is a bivalent or bispecific antibody.

8. The antibody or the antigen-binding fragment thereof of any one of the preceding claims, further comprising a variant Fc constant region.

9. The antibody or antigen-binding fragment thereof of any one of the preceding claims, wherein the antibody is a monoclonal antibody.

10. The antibody or antigen-binding fragment thereof of any one of the preceding claims, wherein the antibody is a chimeric antibody, a humanized antibody, a humanized monoclonal antibody, or a human antibody.

11. The antibody or antigen-binding fragment thereof of any one of the preceding claims, wherein the antibody is a single-chain antibody, a Fab fragment, or a Fab2 fragment.

12. The antibody or antigen-binding fragment thereof of any one of the preceding claims, wherein the antibody or antigen-binding fragment thereof is detectably labeled or conjugated to a toxin, a therapeutic agent, a polymer, a receptor, an enzyme, or a receptor ligand.

13. The antibody or the antigen-binding fragment thereof of claim 12, wherein the polymer is polyethylene glycol (PEG).

14. A pharmaceutical composition comprising the antibody or antigen-binding fragment thereof of any one of the preceding claims and optionally a pharmaceutically acceptable carrier or excipient.

15. The pharmaceutical composition of claim 14, wherein the pharmaceutical comprises two or more of the antibody or antigen-binding fragment thereof of any one of claims 1 to 13.

16. The pharmaceutical composition of any one of claims 14 to 15, further comprising a second therapeutic agent.

17. The pharmaceutical composition of claim 16, wherein the second therapeutic agent comprises an anti-inflammatory agent or an antiviral agent.

18. The pharmaceutical composition of claim 17, wherein the antiviral agent comprises: a nucleoside analog, a peptoid, an oligopeptide, a polypeptide, a protease inhibitor, a 3C-like protease inhibitor, a papain-like protease inhibitor, or an inhibitor of an RNA dependent RNA polymerase.

19. The pharmaceutical composition of claim 18, wherein the antiviral agent is selected from the group consisting of: acyclovir, gancyclovir, vidarabine, foscarnet, cidofovir, amantadine, ribavirin, trifluorothymidine, zidovudine, didanosine, zalcitabine, and an interferon.

20. The pharmaceutical composition of claim 19, wherein the interferon is an interferon-α or an interferon-β.

21. Use of the pharmaceutical composition of any one of claims 14 to 20 in the preparation of a medicament for the diagnosis, prophylaxis, treatment, or combination thereof of a condition resulting from tick-borne flavivirus infection.

22. A nucleic acid molecule encoding a polypeptide chain of the antibody or antigen-binding fragment thereof of any one of claims 1 to 13.

23. A vector comprising the nucleic acid molecule of claim 22.

24. A cultured host cell comprising the vector of claim 23.

25. A method of preparing an antibody, or antigen-binding portion thereof, comprising:
obtaining the cultured host cell of claim 24;
culturing the cultured host cell in a medium under conditions permitting expression of a polypeptide encoded by the vector and assembling of an antibody or fragment thereof; and
purifying the antibody or fragment from the cultured cell or the medium of the cell.

26. A kit comprising a pharmaceutically acceptable dose unit of the antibody or antigen-binding fragment thereof of any one of claims 1 to 13 or the pharmaceutical composition of any one of claims 14 to 20.

27. A kit for the diagnosis, prognosis or monitoring treatment of tick-borne flavivirus infection in a subject, comprising: the antibody or antigen-binding fragment thereof of any one of claims 1 to 13; and a least one detection reagent that binds specifically to the antibody or antigen-binding fragment thereof.

28. A method of neutralizing a tick-borne flavivirus in a subject, comprising administering to a subject in need thereof a therapeutically effective amount of a first antibody or antigen-binding fragment thereof of any one of claims 1 to 13 or a therapeutically effective amount of the pharmaceutical composition of any one of claims 14 to 20.

29. A method of preventing or treating tick-borne flavivirus infection, comprising administering to a subject in need thereof a therapeutically effective amount of a first antibody or antigen-binding fragment thereof of any one of claims 1 to 13 or a therapeutically effective amount of the pharmaceutical composition of any one of claims 14 to 20.

30. The method of claim 28, further comprising administering to the subject a therapeutically effective amount of a second antibody or antigen-binding fragment thereof, wherein the first antibody or antigen-binding fragment thereof and the second antibody or antigen binding fragment thereof exhibit synergistic activity.

31. The method of claim 29, further comprising administering to the subject a therapeutically effective amount of a second antibody or antigen-binding fragment thereof, wherein the first antibody or antigen-binding fragment thereof and the second antibody or antigen binding fragment thereof exhibit synergistic activity.

32. The method of any one of claims 30 to 31, wherein the first antibody or antigen-binding fragment thereof is administered before, after, or concurrently with the second antibody or antigen-binding fragment thereof.

33. The method of any one of claims 28 to 32, further comprising administering to the subject a therapeutically effective amount of a second therapeutic agent or therapy.

34. The method of claim 33, wherein the second therapeutic agent comprises an anti-inflammatory agent or an antiviral agent.

35. The method of claim 34, wherein the antiviral agent comprises: a nucleoside analog, a peptoid, an oligopeptide, a polypeptide, a protease inhibitor, a 3C-like protease inhibitor, a papain-like protease inhibitor, or an inhibitor of an RNA dependent RNA polymerase.

36. The method of claim 34, wherein the antiviral agent is selected from the group consisting of: acyclovir, gancyclovir, vidarabine, foscarnet, cidofovir, amantadine, ribavirin, trifluorothymidine, zidovudine, didanosine, zalcitabine, and an interferon.

37. The method of claim 36, wherein the interferon is an interferon-α or an interferon-β.

38. The method of any one of claims 33 to 37, wherein the antibody or antigen-binding fragment thereof is administered before, after, or concurrently with the second therapeutic agent or therapy.

39. The method of any one of claims 28 to 38, wherein the antibody or antigen-binding fragment thereof is administered to the subject intravenously, subcutaneously, or intraperitoneally.

40. The method of any one of claims 28 to 39, wherein the antibody or antigen-binding fragment thereof is administered prophylactically or therapeutically.

41. A method for detecting the presence of a tick-borne flavivirus in a sample comprising the steps of:
contacting a sample with the antibody or antigen-binding fragment thereof any one of claims 1 to 13; and
determining binding of the antibody or antigen-binding fragment to one or more tick-borne flavivirus antigens,
wherein binding of the antibody to the one or more tick-borne flavivirus antigens is indicative of the presence of the tick-borne flavivirus in the sample.
